SEQUENCE LISTING (55 sequences)

SEQ ID NO: 1 - DNA, 3244 bp, Homo Sapiens
tgacctcggggcaggtcctggtgcagagcgtcgccaaggacgccgagagggaggcgggattgcccagacatccttcagcgaagtgcatgtgtgtttgtaaaccatcgttggctgtcgggagaccgcgaggaccggtccaggctgcggcggagtcgagggcgagggagaggccgcgtgagtgagcagagtccagagccgtgcgcccccagaactgcgcgtccgccccgtgcacccccgcgcgccatgcccagttgccccgcgcgctctgctacgggcccgctctccatcatgggcctcatgccgctcaccaaggaggtggccaagggcagcatcgggcgcggtgtgctccccgccgtggaactggccatcgagcagatccgcaacgagtcactcctgcgcccctacttcctcgacctgcggctctatgacacggagtgcgacaacgcaaaagggttgaaagccttctacgatgcgataaaatacgggccgaaccacttgatggtgtttggaggcgtctgtccatccgtcacatccatcattgcagagtccctccaaggctggaatctggtgcagctttcttttgctgcaaccacgcctgttctagccgataagaaaaaatacccttatttctttcggaccgtcccatcagacaatgcggtgaatccagccattctgaagttgctcaagcactaccagtggaagcgcgtgggcacgctgacgcaagacgttcagaggttctctgaggtgcggaatgacctgactggagttctgtatggcgaggacattgagatttcagacaccgagagcttctccaacgatccctgtaccagtgtcaaaaagctgaaggggaatgatgtgcggatcatccttggccagtttgaccagaatatggcagcaaaagtgttctgttgtgcatacgaggagaacatgtatggtagtaaatatcagtggatcattccgggctggtacgagccttcttggtgggagcaggtgcacacggaagccaactcatcccgctgcctccggaagaatctgcttgctgccatggagggctacattggcgtggatttcgagcccctgagctccaagcagatcaagaccatctcaggaaagactccacagcagtatgagagagagtacaacaacaagcggtcaggcgtggggcccagcaagttccacgggtacgcctacgatggcatctgggtcatcgccaagacactgcagagggccatggagacactgcatgccagcagccggcaccagcggatccaggacttcaactacacggaccacacgctgggcaggatcatcctcaatgccatgaacgagaccaacttcttcggggtcacgggtcaagttgtattccggaatggggagagaatggggaccattaaatttactcaatttcaagacagcagggaggtgaaggtgggagagtacaacgctgtggccgacacactggagatcatcaatgacaccatcaggttccaaggatccgaaccaccaaaagacaagaccatcatcctggagcagctgcggaagatctccctacctctctacagcatcctctctgccctcaccatcctcgggatgatcatggccagtgcttttctcttcttcaacatcaagaaccggaatcagaagctcataaagatgtcgagtccatacatgaacaaccttatcatccttggagggatgctttcctatgcttccatatttctctttggccttgatggatcctttgtctctgaaaagacctttgaaacactttgcaccgtcaggacctggattctcaccgtgggctacacgaccgcttttggggccatgtttgcaaagacctggagagtccacgccatcttcaaaaatgtgaaaatgaagaagaagatcatcaaggaccagaaactgcttgtgatcgtggggggcatgctgctgatcgacctgtgtatcctgatctgctggcaggctgtggaccccctgcgaaggacagtggagaagtacagcatggagccggacccagcaggacgggatatctccatccgccctctcctggagcactgtgagaacacccatatgaccatctggcttggcatcgtctatgcctacaagggacttctcatgttgttcggttgtttcttagcttgggagacccgcaacgtcagcatccccgcactcaacgacagcaagtacatcgggatgagtgtctacaacgtggggatcatgtgcatcatcggggccgctgtctccttcctgacccgggaccagcccaatgtgcagttctgcatcgtggctctggtcatcatcttctgcagcaccatcaccctctgcctggtattcgtgccgaagctcatcaccctgagaacaaacccagatgcagcaacgcagaacaggcgattccagttcactcagaatcagaagaaagaagattctaaaacgtccacctcggtcaccagtgtgaaccaagccagcacatcccgcctggagggcctacagtcagaaaaccatcgcctgcgaatgaagatcacagagctggataaagacttggaagaggtcaccatgcagctgcaggacacaccagaaaagaccacctacattaaacagaaccactaccaagagctcaatgacatcctcaacctgggaaacttcactgagagcacagatggaggaaaggccattttaaaaaatcacctcgatcaaaatccccagctacagtggaacacaacagagccctctcgaacatgcaaagatcctatagaagatataaactctccagaacacatccagcgtcggctgtccctccagctccccatcctccaccacgcctacctcccatccatcggaggcgtggacgccagctgtgtcagcccctgcgtcagccccaccgccagcccccgccacagacatgtgccaccctccttccgagtcatggtctcgggcctgtaagggtgggaggcctgggcccggggcctcccccgtgacagaaccacactgggcagaggggtctgctgcagaaacactgtcggctctggctgcggagaagctgggcaccatggctggcctctcaggaccactcggatggcactcaggtggacaggacggggcagggggagacttggcacctgacctcgagccttatttgtgaagtccttatttcttcacaaagaagaggaacggaaatgggacgtcttccttaacatctgcaaacaaggaggcgctgggatatcaaacttgcaaaaaaaaaaaaaaa

SEQ ID NO: 2 - protein, 898 aa, Homo Sapiens
MPSCPARSATGPLSIMGLMPLTKEVAKGSIGRGVLPAVELAIEQIRNESLLRPYFLDLRLYDTECDNAKGLKAFYDAIKYGPNHLMVFGGVCPSVTSIIAESLQGWNLVQLSFAATTPVLADKKKYPYFFRTVPSDNAVNPAILKLLKHYQWKRVGTLTQDVQRFSEVRNDLTGVLYGEDIEISDTESFSNDPCTSVKKLKGNDVRIILGQFDQNMAAKVFCCAYEENMYGSKYQWIIPGWYEPSWWEQVHTEANSSRCLRKNLLAAMEGYIGVDFEPLSSKQIKTISGKTPQQYEREYNNKRSGVGPSKFHGYAYDGIWVIAKTLQRAMETLHASSRHQRIQDFNYTDHTLGRIILNAMNETNFFGVTGQVVFRNGERMGTIKFTQFQDSREVKVGEYNAVADTLEIINDTIRFQGSEPPKDKTIILEQLRKISLPLYSILSALTILGMIMASAFLFFNIKNRNQKLIKMSSPYMNNLIILGGMLSYASIFLFGLDGSFVSEKTFETLCTVRTWILTVGYTTAFGAMFAKTWRVHAIFKNVKMKKKIIKDQKLLVIVGGMLLIDLCILICWQAVDPLRRTVEKYSMEPDPAGRDISIRPLLEHCENTHMTIWLGIVYAYKGLLMLFGCFLAWETRNVSIPALNDSKYIGMSVYNVGIMCIIGAAVSFLTRDQPNVQFCIVALVIIFCSTITLCLVFVPKLITLRTNPDAATQNRRFQFTQNQKKEDSKTSTSVTSVNQASTSRLEGLQSENHRLRMKITELDKDLEEVTMQLQDTPEKTTYIKQNHYQELNDILNLGNFTESTDGGKAILKNHLDQNPQLQWNTTEPSRTCKDPIEDINSPEHIQRRLSLQLPILHHAYLPSIGGVDASCVSPCVSPTASPRHRHVPPSFRVMVSGL

SEQ ID NO: 3 - DNA, 2823 bp, Rattus Sp.
atggcttccccgccgagctccgggcagccccggccgccgccgccgccgccgccgcccgcgcgcctgctgctgcccctgctgctgtcgctgctgctgtggttggcgcccggggcctggggctggacgcggggcgccccccggccgccgcccagcagcccgccgctctccatcatgggcctcatgccgctcaccaaggaggtggccaagggcagcatcgggcgcggcgtgctccccgccgtggagctagccatcgagcagatccgcaacgagtcactcctgcgcccctacttcctggacctgcgactctatgacaccgagtgtgacaatgcaaagggactgaaagccttctatgacgcaataaagtatgggccgaaccatttgatggtgtttggaggcgtctgtccgtctgtcacatctattatcgcggagtccctccaaggctggaatctggtgcagctttccttcgccgccaccacgcctgttcttgcggataagaagaagtacccgtatttcttccggacggtgccgtcagacaacgcggtgaaccccgccatcctgaagctcctgaagcacttccgctggcggcgtgtgggcacactcacgcaggacgtgcagcgcttctccgaggtgaggaatgacctgactggggttctgtatggggaagatattgagatctcagacacagagagtttctccaatgatccctgcaccagcgtcaaaaagctcaaggggaatgacgtgcggatcatccttggccagtttgaccagaatatggcagcaaaagtcttctgttgtgccttcgaggagagcatgtttggcagcaagtaccagtggatcatcccgggatggtacgagcctgcgtggtgggagcaggtgcatgtggaggccaattcctcacgctgcctgcgcagaagcctcctggctgccatggaaggttacatcggagtggactttgagcccctgagctccaaacaaatcaagaccatctcagggaagactccacagcagtatgaaagagagtacaacagcaaacgttcaggcgtggggcccagcaagttccatgggtacgcctacgatgggatctgggtcatcgccaagaccctacagagggccatggagacactgcatgccagtagcaggcaccagcggatccaggacttcaactacacagaccacacgctgggcaaaatcatcctcaatgccatgaacgagaccaacttcttcggggtcacgggtcaagttgtgttccggaacggggagagaatgggaaccattaaatttactcaatttcaagacagcagagaggtgaaggtcggcgaatacaacgcggtggctgacacactggagatcatcaatgacaccataaggttccaggggtccgagccacccaaggacaagaccatcattctggagcagcttcggaagatctcgcttccactgtatagcatcctgtccgctctcaccatcctcggcatgatcatggccagcgccttcctcttcttcaacatcaagaaccggaaccaaaagctgattaagatgtcaagcccctacatgaacaacctcatcatcctgggaggaatgctgtcctatgcatccatcttcctctttggcctcgatgggtccttcgtctcagaaaagacctttgaaacactctgcacggtccggacctggattctcaccgtgggctacacaactgcctttggggccatgtttgcaaagacctggagggtccatgccatcttcaaaaatgtgaagatgaagaagaagatcatcaaagaccagaagctgcttgtgattgtggggggcatgctgctcatcgacctgtgcatcctgatctgttggcaggctgtggaccccctgcggaggacagtagagaggtacagcatggagccggacccagcaggccgggacatctccatccgcccattgctggaacactgcgaaaacacccacatgaccatctggcttggcattgtctacgcctacaaggggctcctcatgctattcggttgtttcttggcatgggaaacccgcaatgtgagcatccctgccctcaacgacagcaagtacatcggcatgagtgtgtacaatgtggggatcatgtgcatcatcggggctgctgtctccttcctgacgcgtgaccagcccaacgtgcagttctgcatcgtggccctggtcatcatcttctgcagcaccatcactctctgcctggtgtttgtgccaaagctcattactctgaggacaaaccctgacgcagccactcagaacaggcggttccagttcacacagaaccagaagaaagaagattcgaagacctccacttcagtcaccagcgtgaaccaggcgagcacgtcacgcctggagggactgcagtcagaaaaccaccgccttcgaatgaagatcacagagctggacaaagacttggaagaagtcaccatgcagctacaagacacaccagagaagaccacatacatcaaacagaatcactaccaagagctcaacgacatcctcagcttgggcaacttcacagagagcacagatggaggaaaggccattctaaaaaatcacctcgatcaaaacccccagctccagtggaacacgacagagccctcaagaacatgcaaagaccccatagaagacatcaactccccggagcacatccagcgccggctgtcgctccagctccccatccttcaccacgcctacctcccatccatcggaggcgtggatgccagctgcgtcagcccctgtgtcagccctaccgccagccctcgccacagacacgtaccaccctccttccgagtcatggtctcgggcctgtag

SEQ ID NO: 4 - protein, 929 aa, Rattus Sp.
MASPPSSGQPRPPPPPPPPARLLLPLLLSLLLWLAPGAWGWTRGAPRPPPSSPPLSIMGLMPLTKEVAKGSIGRGVLPAVELAIEQIRNESLLRPYFLDLRLYDTECDNAKGLKAFYDAIKYGPNHLMVFGGVCPSVTSIIAESLQGWNLVQLSFAATTPVLADKKKYPYFFRTVPSDNAVNPAILKLLKHFRWRRVGTLTQDVQRFSEVRNDLTGVLYGEDIEISDTESFSNDPCTSVKKLKGNDVRIILGQFDQNMAAKVFCCAFEESMFGSKYQWIIPGWYEPAWWEQVHVEANSSRCLRRSLLAAMEGYIGVDFEPLSSKQIKTISGKTPQQYEREYNSKRSGVGPSKFHGYAYDGIWVIAKTLQRAMETLHASSRHQRIKIILNAMNETNFFGVTGQVVFRNGERMGTIKFTQFQDSREVKVGEYNAVADTLEIINDTIRFQGSEPPKDKTIILEQLRKISLPLYSILSALTILGMIMASAFLFFNIKNRNQKLIKMSSPYMNNLIILGGMLSYASIFLFGLDGSFVSEKTFETLCTVRTWILTVGYTTAFGAMFAKTWRVHAIFKNVKMKKKIIKDQKLLVIVGGMLLIDLCILICWQAVDPLRRTVERYSMEPDPAGRDISIRPLLEHCENTHMTIWLGIVYAYKGLLMLFGCFLAWETRNVSIPALNDSKYIGMSVYNVGIMCIIGAAVSFLTRDQPNVQFCIVALVIIFCSTITLCLVFVPKLITLRTNPDAATQNRRFQFTQNQKKEDSKTSTSVTSVNQASTSRLEGLQSENHRLRMKITELDKDLEEVTMQLQDTPEKTTYIKQNHYQELNDILSLGNFTESTDGGKAILKNHLDQNPQLQWNTTEPSRTCKDPIEDINSPEHIQRRLSLQLPILHHAYLPSIGGVDASCVSPCVSPTASPRHRHVPPSFRVMVSGL

SEQ ID NO: 5 - DNA, 45 bp, Artificial Sequence
  Description of Artificial Sequence Oligonucleotide Probe
agggatgctttcctatgcttccatatttctctttggccttgatgg

SEQ ID NO: 6 - DNA, 45 bp, Artificial Sequence
  Description of Artificial Sequence Oligonucleotide Probe
caatgtgcagttctgcatcgtggctctggtcatcatcttctgcag

SEQ ID NO: 7 - DNA, 24 bp, Artificial Sequence
  Description of Artificial Sequence Primer
cttctaggcctgtacggaagtgtt

SEQ ID NO: 8 - DNA, 26 bp, Artificial Sequence
  Description of Artificial Sequence Primer
gttgtggtttgtccaaactcatcaat

SEQ ID NO: 9 - DNA, 24 bp, Artificial Sequence
  Description of Artificial Sequence Primer
gggatgagtgtctacaacgtgggg

SEQ ID NO: 10 - DNA, 26 bp, Artificial Sequence
  Description of Artificial Sequence Primer
tgcgttgctgcatctgggtttgttct

SEQ ID NO: 11 - DNA, 26 bp, Artificial Sequence
  Description of Artificial Sequence Primer
atctccctacctctctacagcatcct

SEQ ID NO: 12 - DNA, 26 bp, Artificial Sequence
  Description of Artificial Sequence Primer
caggtcctgacggtgcaaagtgtttc

SEQ ID NO: 13 - DNA, 26 bp, Artificial Sequence
  Description of Artificial Sequence Primer
tgacgcaagacgttcagaggttctct

SEQ ID NO: 14 - DNA, 26 bp, Artificial Sequence
  Description of Artificial Sequence Primer
tgtagccttccatggcagcaagcaga

SEQ ID NO: 15 - DNA, 26 bp, Artificial Sequence
  Description of Artificial Sequence Primer
agagaacctctgaacgtcttgcgtca

SEQ ID NO: 16 - DNA, 26 bp, Artificial Sequence
  Description of Artificial Sequence Primer
ggctctgttgtgttccactgtagctg

SEQ ID NO: 17 - DNA, 26 bp, Artificial Sequence
  Description of Artificial Sequence Primer
tcatgccgctcaccaaggaggtggcc

SEQ ID NO: 18 - DNA, 26 bp, Artificial Sequence
  Description of Artificial Sequence Primer
ggccacctccttggtgagcggcatga

SEQ ID NO: 19 - DNA, 24 bp, Artificial Sequence
  Description of Artificial Sequence Primer
tgagtgagcagagtccagagccgt

SEQ ID NO: 20 - DNA, 26 bp, Artificial Sequence
  Description of Artificial Sequence Primer
atggatgggaggtaggcgtggtggag

SEQ ID NO: 21 - DNA, 26 bp, Artificial Sequence
  Description of Artificial Sequence Primer
ctctctgccctcaccatcctcgggat

SEQ ID NO: 22 - DNA, 26 bp, Artificial Sequence
  Description of Artificial Sequence Primer
gactccggctcgaataccaggcagag

SEQ ID NO: 23 - DNA, 27 bp, Artificial Sequence
  Description of Artificial Sequence Primer
ccatgtttgcaaagacctggagggtcc

SEQ ID NO: 24 - DNA, 27 bp, Artificial Sequence
  Description of Artificial Sequence Primer
ggtcacgcgtcaggaaagagacagcag

SEQ ID NO: 25 - DNA, 25 bp, Artificial Sequence
  Description of Artificial Sequence Primer
aagcttctagagatccctcgacctc

SEQ ID NO: 26 - DNA, 25 bp, Artificial Sequence
  Description of Artificial Sequence Primer
aggcgcagaactggtaggtatggaa

SEQ ID NO: 27 - DNA, 25 bp, Artificial Sequence
  Description of Artificial Sequence Primer
cttctaggcctgtacggaagtgtta

SEQ ID NO: 28 - DNA, 27 bp, Artificial Sequence
  Description of Artificial Sequence Primer
gttgtggtttgtccaaactcatcaatg

SEQ ID NO: 29 - DNA, 27 bp, Artificial Sequence
  Description of Artificial Sequence Primer
ctgctgtctctttcctgacgcgtgacc

SEQ ID NO: 30 - DNA, 59 bp, Artificial Sequence
  Description of Artificial Sequence Primer
ccaagcttctaatacgactcactataggggagaccatgggcccggggggaccctgtacc

SEQ ID NO: 31 - DNA, 63 bp, Artificial Sequence
  Description of Artificial Sequence Primer
tttttttttttttttttttttttttttttttttttcacttgtaaagcaaatgtactcgactcc

SEQ ID NO: 32 - DNA, 37 bp, Artificial Sequence
  Description of Artificial Sequence Primer
cgcggatccattatgtctgcactccgaaggaaatttg

SEQ ID NO: 33 - DNA, 38 bp, Artificial Sequence
  Description of Artificial Sequence Primer
cgcgaattcttatgtgaagcgatcagagttcatttttc

SEQ ID NO: 34 - DNA, 34 bp, Artificial Sequence
  Description of Artificial Sequence Primer
gcgggatccgctatggctggtgattctaggaatg

SEQ ID NO: 35 - DNA, 29 bp, Artificial Sequence
  Description of Artificial Sequence Primer
ccggaattcccctcacaccgagcccctgg

SEQ ID NO: 36 - DNA, 44 bp, Artificial Sequence
  Description of Artificial Sequence Oligonucleotide Probe
gcaataaagtatgggctgaaccatttgatggtgtttggaggcgt

SEQ ID NO: 37 - DNA, 44 bp, Artificial Sequence
  Description of Artificial Sequence Oligonucleotide Probe
acgcctccaaacaccatcaaatggttcagcccatactttattgc

SEQ ID NO: 38 - DNA, 40 bp, Artificial Sequence
  Description of Artificial Sequence Oligonucleotide Probe
tttgagcccctgagctccaaacaaatcaagaccatctcag

SEQ ID NO: 39 - DNA, 40 bp, Artificial Sequence
  Description of Artificial Sequence Oligonucleotide Probe
ctgagatggtcttgatttgtttggagctcaggggctcaaa

SEQ ID NO: 40 - DNA, 43 bp, Artificial Sequence
  Description of Artificial Sequence Oligonucleotide Probe
aaggccatcaacttcctgcctgtggactatgagatcgaatatg

SEQ ID NO: 41 - DNA, 43 bp, Artificial Sequence
  Description of Artificial Sequence Oligonucleotide Probe
catattcgatctcatagtccacaggcaggaagttgatggcctt

SEQ ID NO: 42 - DNA, 38 bp, Artificial Sequence
  Description of Artificial Sequence Oligonucleotide Probe
tggccgctgcctcttctgctggtgatggcggctggggt

SEQ ID NO: 43 - DNA, 38 bp, Artificial Sequence
  Description of Artificial Sequence Oligonucleotide Probe
accccagccgccatcaccagcagaagaggcagcggcca

SEQ ID NO: 44 - DNA, 45 bp, Artificial Sequence
  Description of Artificial Sequence Oligonucleotide Probe
ccttggctttggccttgaacaagacgtctggaggaggtggtcgtt

SEQ ID NO: 45 - DNA, 45 bp, Artificial Sequence
  Description of Artificial Sequence Oligonucleotide Probe
aacgaccacctcctccagacgtcttgttcaaggccaaagccaagg

SEQ ID NO: 46 - DNA, 2826 bp, Homo Sapiens
atggcttccccgcggagctccgggcagcccgggccgccgccgccgccgccaccgccgcccgcgcgcctgctactgctactgctgctgccgctgctgctgcctctggcgcccggggcctggggctgggcgcggggcgccccccggccgccgcccagcagcccgccgctctccatcatgggcctcatgccgctcaccaaggaggtggccaagggcagcatcgggcgcggtgtgctccccgccgtggaactggccatcgagcagatccgcaacgagtcactcctgcgcccctacttcctcgacctgcggctctatgacacggagtgcgacaacgcaaaagggttgaaagccttctacgatgcgataaaatacgggccgaaccacttgatggtgtttggaggcgtctgtccatccgtcacatccatcattgcagagtccctccaaggctggaatctggtgcagctttcttttgctgcaaccacgcctgttctagccgataagaaaaaatacccttatttctttcggaccgtcccatcagacaatgcggtgaatccagccattctgaagttgctcaagcactaccagtggaagcgcgtgggcacgctgacgcaagacgttcagaggttctctgaggtgcggaatgacctgactggagttctgtatggcgaggacattgagatttcagacaccgagagcttctccaacgatccctgtaccagtgtcaaaaagctgaaggggaatgatgtgcggatcatccttggccagtttgaccagaatatggcagcaaaagtgttctgttgtgcatacgaggagaacatgtatggtagtaaatatcagtggatcattccgggctggtacgagccttcttggtgggagcaggtgcacacggaagccaactcatcccgctgcctccggaagaatctgcttgctgccatggagggctacattggcgtggatttcgagcccctgagctccaagcagatcaagaccatctcaggaaagactccacagcagtatgagagagagtacaacaacaagcggtcaggcgtggggcccagcaagttccacgggtacgcctacgatggcatctgggtcatcgccaagacactgcagagggccatggagacactgcatgccagcagccggcaccagcggatccaggacttcaactacacggaccacacgctgggcaggatcatcctcaatgccatgaacgagaccaacttcttcggggtcacgggtcaagttgtattccggaatggggagagaatggggaccattaaatttactcaatttcaagacagcagggaggtgaaggtgggagagtacaacgctgtggccgacacactggagatcatcaatgacaccatcaggttccaaggatccgaaccaccaaaagacaagaccatcatcctggagcagctgcggaagatctccctacctctctacagcatcctctctgccctcaccatcctcgggatgatcatggccagtgcttttctcttcttcaacatcaagaaccggaatcagaagctcataaagatgtcgagtccatacatgaacaaccttatcatccttggagggatgctttcctatgcttccatatttctctttggccttgatggatcctttgtctctgaaaagacctttgaaacactttgcaccgtcaggacctggattctcaccgtgggctacacgaccgcttttggggccatgtttgcaaagacctggagagtccacgccatcttcaaaaatgtgaaaatgaagaagaagatcatcaaggaccagaaactgcttgtgatcgtggggggcatgctgctgatcgacctgtgtatcctgatctgctggcaggctgtggaccccctgcgaaggacagtggagaagtacagcatggagccggacccagcaggacgggatatctccatccgccctctcctggagcactgtgagaacacccatatgaccatctggcttggcatcgtctatgcctacaagggacttctcatgttgttcggttgtttcttagcttgggagacccgcaacgtcagcatccccgcactcaacgacagcaagtacatcgggatgagtgtctacaacgtggggatcatgtgcatcatcggggccgctgtctccttcctgacccgggaccagcccaatgtgcagttctgcatcgtggctctggtcatcatcttctgcagcaccatcaccctctgcctggtattcgtgccgaagctcatcaccctgagaacaaacccagatgcagcaacgcagaacaggcgattccagttcactcagaatcagaagaaagaagattctaaaacgtccacctcggtcaccagtgtgaaccaagccagcacatcccgcctggagggcctacagtcagaaaaccatcgcctgcgaatgaagatcacagagctggataaagacttggaagaggtcaccatgcagctgcaggacacaccagaaaagaccacctacattaaacagaaccactaccaagagctcaatgacatcctcaacctgggaaacttcactgagagcacagatggaggaaaggccattttaaaaaatcacctcgatcaaaatccccagctacagtggaacacaacagagccctctcgaacatgcaaagatcctatagaagatataaactctccagaacacatccagcgtcggctgtccctccagctccccatcctccaccacgcctacctcccatccatcggaggcgtggacgccagctgtgtcagcccctgcgtcagccccaccgccagcccccgccacagacatgtgccaccctccttccgagtcatggtctcgggcctgtaa

SEQ ID NO: 47 - protein, 941 aa, Homo Sapiens
MASPRSSGQPGPPPPPPPPPARLLLLLLLPLLLPLAPGAWGWARGAPRPPPSSPPLSIMGLMPLTKEVAKGSIGRGVLPAVELAIEQIRNESLLRPYFLDLRLYDTECDNAKGLKAFYDAIKYGPNHLMVFGGVCPSVTSIIAESLQGWNLVQLSFAATTPVLADKKKYPYFFRTVPSDNAVNPAILKLLKHYQWKRVGTLTQDVQRFSEVRNDLTGVLYGEDIEISDTESFSNDPCTSVKKLKGNDVRIILGQFDQNMAAKVFCCAYEENMYGSKYQWIIPGWYEPSWWEQVHTEANSSRCLRKNLLAAMEGYIGVDFEPLSSKQIKTISGKTPQQYEREYNNKRSGVGPSKFHGYAYDGIWVIAKTLQRAMETLHASSRHQRIQDFNYTDHTLGRIILNAMNETNFFGVTGQVVFRNGERMGTIKFTQFQDSREVKVGEYNAVADTLEIINDTIRFQGSEPPKDKTIILEQLRKISLPLYSILSALTILGMIMASAFLFFNIKNRNQKLIKMSSPYMNNLIILGGMLSYASIFLFGLDGSFVSEKTFETLCTVRTWILTVGYTTAFGAMFAKTWRVHAIFKNVKMKKKIIKDQKLLVIVGGMLLIDLCILICWQAVDPLRRTVEKYSMEPDPAGRDISIRPLLEHCENTHMTIWLGIVYAYKGLLMLFGCFLAWETRNVSIPALNDSKYIGMSVYNVGIMCIIGAAVSFLTRDQPNVQFCIVALVIIFCSTITLCLVFVPKLITLRTNPDAATQNRRFQFTQNQKKEDSKTSTSVTSVNQASTSRLEGLQSENHRLRMKITELDKDLEEVTMQLQDTPEKTTYIKQNHYQELNDILNLGNFTESTDGGKAILKNHLDQNPQLQWNTTEPSRTCKDPIEDINSPEHIQRRLSLQLPILHHAYLPSIGGVDASCVSPCVSPTASPRHRHVPPSFRVMVSGL

SEQ ID NO: 48 - protein, 27 aa, Artificial Sequence
  Description of Artificial Sequence Peptide
PLYSILSALTILGMIMASAFLFFNIKN

SEQ ID NO: 49 - protein, 27 aa, Artificial Sequence
  Description of Artificial Sequence Peptide
LIILGGMLSYASIFLFGLDGSFVSEKT

SEQ ID NO: 50 - protein, 25 aa, Artificial Sequence
  Description of Artificial Sequence Peptide
CTVRTWILTVGYTTAFGAMFAKTWR

SEQ ID NO: 51 - protein, 22 aa, Artificial Sequence
  Description of Artificial Sequence Peptide
QKLLVIVGGMLLIDLCILICWQ

SEQ ID NO: 52 - protein, 24 aa, Artificial Sequence
  Description of Artificial Sequence Peptide
MTIWLGIVYAYKGLLMLFGCFLAW

SEQ ID NO: 53 - protein, 25 aa, Artificial Sequence
  Description of Artificial Sequence Peptide
ALNDSKYIGMSVYNVGIMCIIGAAV

SEQ ID NO: 54 - protein, 25 aa, Artificial Sequence
  Description of Artificial Sequence Peptide
ALNDSKYIGMSVYNVGIMCIIGAAV

SEQ ID NO: 55 - protein, 844 aa, Rattus Sp.
MGPGGPCTPVGWPLPLLLVMAAGVAPVWASHSPHLPRPHPRVPPHPSSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKTYDPSINCTVEEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRSQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSKKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSETQDTMKTGSSTNNNEEEKSRLLEKENRELEKIIAEKEERVSELRHQLQSRQQLRSRRHPPTPPDPSGGLPRGPSEPPDRLSCDGSRVHLLYK

(19) United States
(12) Patent Application Publication
(10) Pub. No.: US 2002/0156265 A1
(43) Pub. Date: Oct. 24, 2002

(54) DNA encoding a GABABR2 polypeptide and uses thereof

(76) Inventors: Kenneth A. Jones, Waltham, MA (US); Thomas M. Laz, Parlin, NJ (US); Beth Borowsky, Montclair, NJ (US)

Correspondence Address: Cooper & Dunham LLP, 1185 Avenue of the Americas, New York, NY, 10036, US

(21) Appl. No.: 09/793,139

(22) Filed: Feb. 26, 2001

Related U.S. Application Data
(63) Continuation of application No. PCT/US99/19651, filed on Aug. 27, 1999.
Application No. PCT/US99/19651 is a continuation-in-part of application No. 09/211,755, filed on Dec. 15, 1998.
Application No. 09/211,755 is a continuation-in-part of application No. 09/186,664, filed on Nov. 4, 1998.
Application No. 09/186,664 is a continuation-in-part of application No. PCT/US98/22033, filed on Oct. 16, 1998.
Application No. PCT/US98/22033 is a continuation-in-part of application No. 09/141,760, filed on Aug. 27, 1998, now abandoned.

(30) Foreign Application Priority Data
Aug. 27, 1999 (US) PCT/US99/19651

(51) Int. Cl. C07H 21/04; C12Q 1/68; C12P 21/02; C07K 14/705

(52) U.S. Cl. 536/23.5; 435/6; 435/69.1; 530/350

(57) ABSTRACT

This invention provides isolated nucleic acids encoding a mammalian GABA_{B}R2 polypeptide, an isolated GABA_{B}R2 protein, vectors comprising isolated nucleic acid encoding mammalian GABA_{B}R2 polypeptides, cells expressing mammalian GABA_{B}R1/R2 receptors, antibodies directed to an epitope on mammalian GABA_{B}R2 polypeptides or mammalian GABA_{B}R1/R2 receptors, nucleic acid probes useful for detecting nucleic acids encoding mammalian GABA_{B}R2 polypeptides, antisense oligonucleotides complementary to unique sequences of nucleic acids encoding mammalian GABA_{B}R2 polypeptides, nonhuman transgenic animals which express DNA encoding normal or mutant mammalian GABA_{B}R1/R2 receptors, as well as methods of screening compounds acting as agonists or antagonists of mammalian GABA_{B}R1/R2 receptors.

### BACKGROUND OF THE INVENTION

This application is a continuation-in-part of U.S. Ser. No. 09/211,755, filed Dec. 15, 1998 which is a continuation-in-part of U.S. Ser. No. 09/186,664, filed Nov. 4, 1998 which is a continuation-in-part of PCT International Application No. PCT/US98/22033, filed Oct. 16, 1998 which is a continuation-in-part of U.S. Ser. No. 09/141,760, filed Aug. 27, 1998, which is a continuation-in-part of U.S. Ser. No. 08/953,277, filed Oct. 17, 1997, the contents of which are hereby incorporated by reference into the subject application.

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the sequence listing and the claims.

Gamma amino butyric acid (GABA) is the major inhibitory neurotransmitter in the nervous system. Three families of receptors for this neurotransmitter, GABA_{A}, GABA_{B}, and GABA_{C}, have been defined pharmacologically and genetically. GABA_{B} receptors were initially discriminated by their sensitivity to the drug baclofen (Bowery, 1993). This and their dependency on G-proteins for effector coupling distinguishes them from the ion channel-forming GABA_{A} and GABA_{C} receptors. Principle molecular targets of GABA_{B} receptor activation are Ca⁺⁺ and K⁺ channels whose gating is directly modulated by the liberation of G-protein that follows the binding of the neurotransmitter to its receptor (Misgeld et al. 1995; Krapivinsky et al., 1995a).

In this sense, GABA_{B} receptors operate mechanistically as other G-protein coupled receptors (GPCRs), such as dopamine D2, serotonin 5HT1a, neuropeptide Y and opiate receptors, that are also negatively coupled to adenylyl cyclase activity (North, 1989). Stimulation of GABA_{B} receptors inhibits release of neurotransmitters such as glutamate, GABA, somatostatin, and acetylcholine by modulation of Ca⁺⁺ and K⁺ channels at presynaptic nerve terminals. Inhibition of neurotransmitter release is one of the most prominent physiological actions of the GABA_{B} receptor and has provided a basis for the discrimination of receptor subtypes (Bowery et al. 1990). GABA_{B} receptors also mediate a powerful postsynaptic hyperpolarization of neuronal cell bodies via the opening of G-protein-gated inwardly rectifying K⁺ channels (GIRK) (Kofuji et al. 1996).

GABA_{B} receptors are widely distributed throughout the central nervous system. Receptor autoradiography and binding studies show that receptors are found in relatively high abundance in nearly all areas of the brain including cerebral cortex, hippocampus, cerebellum, basal ganglia, thalamus, and spinal cord (Bowery et al. 1987). In the periphery, GABA and GABA_{B} receptors are found in pancreatic islets, autonomic ganglia, guinea-pig ileum, lung, oviduct, and urinary bladder (Giotti et al. 1983; Erdo et al. 1984; Santicioli et al. 1986; Sawynok, 1986; Hills et al. 1989; Chapman et al. 1993).

Baclofen, the agonist that originally defined the GABA_{B} receptor subtype, has been used as an anti-spastic agent for the past 25 years. There is evidence in human that baclofen has a spinal site of action that most likely involves the depression of mono-and polysynaptic reflexes. In laboratory animals, baclofen has antinociceptive properties that are attributed to the inhibition of release of excitatory neurotransmitters glutamate and substance P from primary sensory afferent terminals (Dirig and Yaksh, 1978; Sawynok, 1987; Malcangio et al., 1991). The presence of GABA_{B} receptors in intestine, lung and urinary bladder indicates a possible therapeutic role for diseases associated with these peripheral tissues. In spinal patients, baclofen is currently used for treatment of bladder-urethral dissynergia (Leyson et al., 1980). Selective GABA_{B} receptor agonists may also prove useful for the treatment of incontinence by reducing the feeling of bladder fullness (Taylor and Bates, 1979). Evidence from studies of the upper respiratory systems of cats and guinea-pigs suggests that GABA_{B} agonists also may be useful as antitussive agents and for the treatment of asthma (Luzzi et al., 1987; Bolser et al., 1993). In addition, GABA_{B} receptors have been implicated in absence seizure activity in the neocortex and with presynaptic depression of excitatory transmission in the spinal cord.

Studies of GABA_{B} receptor pharmacology and physiology have been greatly facilitated by the relatively recent arrival of potent and selective GABA_{B} receptor antagonists that are able to penetrate the blood-brain barrier. The most fruitful avenue for providing glimpses of GABA_{B} receptor subtypes has come from studies of neurotransmitter release. GABA, acting through GABA_{B} receptors, can inhibit the release of GABA, glutamate, and somatostatin in rat cerebrocortical synaptosomes depolarized with KCl. Three receptor subtypes have been hypothesized based on the potency of the agonists baclofen and 3-aminopropylphosphinic acid (3-APPA), and on the antagonists phaclofen and CGP35348 (Bonanno, Raiteri, 1992). For example, somatostatin release is inhibited by baclofen and this effect is antagonized by phaclofen and CGP35348. Glutamate release is similarly affected except that the potency of phaclofen to block inhibition is considerably lower than that for release of somatostatin. A third receptor subtype, the cortical GABA autoreceptor, has been defined based on an insensitivity to CGP35348, although this potency difference is not seen in a cortical slice preparation (Waldmeier et al. 1994). In the spinal cord, the GABA autoreceptor is insensitive to baclofen, but sensitive to 3APPA and block by CGP35348.

Interestingly, in this tissue baclofen is active at the GABA_{B} receptor modulating glutamate release. Differences in the sensitivities of presynaptic receptors controlling release of GABA and glutamate in the spinal cord may importantly contribute to the therapeutic action of baclofen as an antispastic agent (Bonanno, Raiteri, 1993).

Recently a polypeptide was isolated, GABA_{B}R1a, that binds radiolabelled GABA_{B} receptor antagonists in transfected cells (Kaupmann et al. 1997a). The predicted amino acid sequence displays homology with the metabotropic glutamate receptor gene family which includes eight members and a Ca⁺⁺-sensing receptor. Included in this homology is a large N-terminal domain that contains two lobes with structural similarity to the amino acid binding sites of bacterial proteins. A second polypeptide, GABA_{B}R1b, presumably a splice variant, differs from GABA_{B}R1a in that the N-terminal 147 amino acids are replaced by 18 different residues in the predicted mature protein after signal peptide cleavage. Transcripts for both GABA_{B}R1s are abundant and widely distributed in the rat brain. There appear to be differences in the localization of the splice variants in discrete regions of the brain, suggesting that their expression is differentially regulated (Bischoff et al. 1997).

The pharmacological profile of the cloned GABA_{B}R1 polypeptide is similar in some respects to that of native receptors isolated from rat cerebral cortex, but there are important differences. For the high affinity antagonists studied, IC₅₀s are nearly identical to those at native receptors. In contrast, IC₅₀s for agonists and some low affinity antagonists display large rightward shifts relative to their displacement curves in native tissue. Additionally, both splice variants of the polypeptide couple poorly to intracellular effectors such as inhibition of adenylyl cyclase and, against expectations, fail completely to stimulate GIRK currents in oocytes (Kaupmann et al. 1997b). The poor binding affinity of agonists and weak or non-existent activation of effectors may not be adequately explained by inappropriate G-protein coupling in the heterologous expression system used.

The isolation by homology cloning of a novel polypeptide, GABA_{B}R2, from a human hippocampus cDNA library, as well the isolation of the rat homolog of the human polypeptide, is now reported. Also reported herein are functional assays involving the co-expression of the GABA_{B}R2 gene with a GABA_{B}R1 gene. These functional assays were not previously observed with the GABA_{B}R1 gene product alone. The pharmacological and signal transduction properties of the two gene products when expressed together match those of native GABA_{B} receptors in the brain. These functional assays permits high throughput screening for novel compounds having agonist or antagonist activity at the native GABA_{B} receptor.

### SUMMARY OF THE INVENTION

This invention is directed to an isolated nucleic acid encoding a GABA_{B}R2 polypeptide.

This invention is further directed to a purified GABA_{B}R2 protein.

This invention is further directed to a vector comprising the above-identified nucleic acid.

This invention is further directed to a above-identified vector, wherein the vector is a plasmid.

This invention is directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within one of the two strands of the nucleic acid encoding the GABA_{B}R2 polypeptide contained in plasmid BO-55, and detecting hybridization of the probe to the nucleic acid.

This invention is further directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within (a) the nucleic acid sequence shown in FIGS. 22A-22D (SEQ ID NO:46) or (b) the reverse complement to the nucleic acid sequence shown in FIGS. 22A-22D (SEQ ID NO:46), and detecting hybridization of the probe to the nucleic acid.

This invention is further directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within one of the two strands of the nucleic acid encoding the GABA_{B}R2 polypeptide contained in plasmid pEXJT3T7-hGABAB2, and detecting hybridization of the probe to the nucleic acid.

This invention is further directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within (a) the nucleic acid sequence shown in FIGS. 3A-3D (SEQ ID NO:3) or (b) the reverse complement to the nucleic acid sequence shown in FIGS. 3A-3D (SEQ ID NO:3), and detecting hybridization of the probe to the nucleic acid.

This invention is further directed to a method of detecting a nucleic acid encoding a GABA_{B}R2 polypeptide, which comprises contacting the nucleic acid with a probe comprising a nucleic acid of at least 15 nucleotides which is complementary to the antisense sequence of a unique segment of the sequence of the nucleic acid encoding the GABA_{B}R2 polypeptide, and detecting hybridization of the probe to the nucleic acid.

This invention is directed to an isolated antibody capable of binding to a GABA_{B}R2 polypeptide encoded by the above-identified nucleic acid.

This invention is further directed to an antibody capable of competitively inhibiting the binding of the above-identified antibody to a GABA_{B}R2 polypeptide.

This invention is further directed to a pharmaceutical composition which comprises an amount of the above-identified antibody effective to block binding of a ligand to the GABA_{B}R2 polypeptide and a pharmaceutically acceptable carrier.

This invention is directed to a transgenic, nonhuman mammal expressing DNA encoding a GABA_{B}R2 polypeptide.

This invention is further directed to a transgenic, nonnuman mammal comprising a homologous recombination knockout of the native GABA_{B}R2 polypeptide.

This invention is further directed to a transgenic, nonhuman mammal whose genome comprises antisense DNA complementary to DNA encoding an above-identified GABA_{B}R2 polypeptide so placed as to be transcribed into antisense mRNA which is complementary to mRNA encoding such GABA_{B}R2 polypeptide and which hybridizes to such mRNA encoding such GABA_{B}R2 polypeptide, thereby reducing its translation.

This invention is directed to a method of detecting the presence of a GABA_{B}R2 polypeptide on the surface of a cell which comprises contacting the cell with the above-identified antibody under conditions permitting binding of the antibody to the polypeptide, detecting the presence of the antibody bound to the cell, and thereby detecting the presence of a GABA_{B}R2 polypeptide on the surface of the cell.

This invention is further directed to a method of preparing the purified GABA_{B}R2 polypeptide which comprises:

a. inducing cells to express a GABA_{B}R2 polypeptide;

b. recovering the polypeptide so expressed from the induced cells; and

c. purifying the polypeptide so recovered.

This invention is further directed to a method of preparing the purified GABA_{B}R2 polypeptide which comprises:

a. inserting a nucleic acid encoding the GABA_{B}R2 polypeptide into a suitable vector;

b. introducing the resulting vector in a suitable host cell;

c. placing the resulting cell in suitable condition permitting the production of the GABA_{B}R2 polypeptide;

d. recovering the polypeptide produced by the resulting cell; and

e. isolating or purifying the polypeptide so recovered.

This invention is directed to a GABA_{B}R1/R2 receptor comprising two polypeptides, one of which is a GABA_{B}R2 polypeptide and another of which is a GABA_{B}R1 polypeptide.

This invention is directed to a method of forming a GABA_{B}R1/R2 receptor which comprises inducing cells to express both a GABA_{B}R1 polypeptide and a GABA_{B}R2 polypeptide.

This invention is directed to an antibody capable of binding to a GABA_{B}R1/R2 receptor, wherein the GABA_{B}R2 polypeptide is encoded by the above-identified nucleic acid.

This invention is further directed to an antibody capable of competitively inhibiting the binding of the above-identified antibody to a GABA_{B}R1/R2 receptor.

This invention is directed to a pharmaceutical composition which comprises an amount of the above-identified antibody effective to block binding of a ligand to the GABA_{B}R1/R2 receptor and a pharmaceutically acceptable carrier.

This invention is directed to a transgenic, nonhuman mammal expressing a GABA_{B}R1/R2 receptor, which is not naturally expressed by the mammal.

This invention is further directed to a transgenic, nonhuman mammal comprising a homologous recombination knockout of the native GABA_{B}R1/R2 receptor.

This invention is directed to a method of detecting the presence of a GABA_{B}R1/R2 receptor on the surface of a cell which comprises contacting the cell with the above-identified antibody under conditions permitting binding of the antibody to the receptor, detecting the presence of the antibody bound to the cell, and thereby detecting the presence of a GABA_{B}R1/R2 receptor on the surface of the cell.

This invention is directed to a method of determining the physiological effects of varying levels of activity of GABA_{B}R1/R2 receptors which comprises producing an above-identified transgenic nonhuman mammal whose levels of GABA_{B}R1/R2 receptor activity vary due to the presence of an inducible promoter which regulates GABA_{B}R1/R2 receptor expression.

This invention is directed to a cell which expresses on its surface a mammalian GABA_{B}R1/R2 receptor that is not naturally expressed on the surface of such cell.

This invention is directed to a process for identifying a chemical compound which specifically binds to a GABA_{B}R1/R2 receptor which comprises contacting cells containing nucleic acid encoding and expressing on their cell surface the GABA_{B}R1/R2 receptor, wherein such cells do not normally express the GABA_{B}R1/R2 receptor, with the compound under conditions suitable for binding, and detecting specific binding of the chemical compound to the GABA_{B}R1/R2 receptor.

This invention is directed to a process for identifying a chemical compound which specifically binds to a GABA_{B}R1/R2 receptor which comprises contacting a membrane fraction from a cell extract of cells containing nucleic acid encoding and expressing on their cell surface the GABA_{B}R1/R2 receptor, wherein such cells do not normally express the GABA_{B}R1/R2 receptor, with the compound under conditions suitable for binding, and detecting specific binding of the chemical compound to the GABA_{B}R1/R2 receptor.

This invention is directed to a process involving competitive binding for identifying a chemical compound which specifically binds to a GABA_{B}R1/R2 receptor which comprises separately contacting cells expressing on their cell surface the GABA_{B}R1/R2 receptor, wherein such cells do not normally express the GABA_{B}R1/R2 receptor, with both the chemical compound and a second chemical compound known to bind to the receptor, and with only the second chemical compound, under conditions suitable for binding of both compounds, and detecting specific binding of the chemical compound to the GABA_{B}R1/R2 receptor, a decrease in the binding of the second chemical compound to the GABA_{B}R1/R2 receptor in the presence of the chemical compound indicating that the chemical compound binds to the GABA_{B}R1/R2 receptor.

This invention is directed to a process involving competitive binding for identifying a chemical compound which specifically binds to a human GABA_{B}R1/R2 receptor which comprises separately contacting a membrane fraction from a cell extract of cells expressing on their cell surface the GABA_{B}R1/R2 receptor, wherein such cells do not normally express the GABA_{B}R1/R2 receptor, with both the chemical compound and a second chemical compound known to bind to the receptor, and with only the second chemical compound, under conditions suitable for binding of both compounds, and detecting specific binding of the chemical compound to the GABA_{B}R1/R2 receptor, a decrease in the binding of the second chemical compound to the GABA_{B}R1/R2 receptor in the presence of the chemical compound indicating that the chemical compound binds to the GABA_{B}R1/R2 receptor.

This invention is directed to a method of screening a plurality of chemical compounds not known to bind to a GABA_{B}R1/R2 receptor to identify a compound which specifically binds to the GABA_{B}R1/R2 receptor, which comprises

(a) contacting cells containing nucleic acid encoding and expressing on their cell surface the GABA_{B}R1/R2 receptor, wherein such cells do not normally express the GABA_{B}R1/R2 receptor, with a compound known to bind specifically to the GABA_{B}R1/R2 receptor;

(b) contacting the same cells as in step (a) with the plurality of compounds not known to bind specifically to the GABA_{B}R1/R2 receptor, under conditions permitting binding of compounds known to bind the GABA_{B}R1/R2 receptor;

(c) determining whether the binding of the compound known to bind specifically to the GABA_{B}R1/R2 receptor is reduced in the presence of the plurality of the compounds, relative to the binding of the compound in the absence of the plurality of compounds, and if the binding is reduced;

(d) separately determining the extent of binding to the GABA_{B}R1/R2 receptor of each compound included in the plurality of compounds, so as to thereby identify the compound or compounds present in such plurality of compounds which specifically binds to the GABA_{B}R1/R2 receptor.

This invention is directed to a method of screening a plurality of chemical compounds not known to bind to a GABA_{B}R1/R2 receptor to identify a compound which specifically binds to the GABA_{B}R1/R2 receptor, which comprises

(a) contacting a membrane fraction extract from cells containing nucleic acid encoding and expressing on their cell surface the GABA_{B}R1/R2 receptor, wherein such cells do not normally express the GABA_{B}R1/R2 receptor, with a compound known to bind specifically to the GABA_{B}R1/R2 receptor;

(b) contacting the same membrane fraction as in step (a) with the plurality of compounds not known to bind specifically to the GABA_{B}R1/R2 receptor, under conditions permitting binding of compounds known to bind the GABA_{B}R1/R2 receptor;

(c) determining whether the binding of the compound known to bind specifically to the GABA_{B}R1/R2 receptor is reduced in the presence of the plurality of compounds, relative to the binding of the compound in the absence of the plurality of compounds, and if the binding is reduced;

(d) separately determining the extent of binding to the GABA_{B}R1/R2 receptor of each compound included in the plurality of compounds, so as to thereby identify the compound or compounds present in such plurality of compounds which specifically binds to the GABA_{B}R1/R2 receptor.

This invention is directed to a process for determining whether a chemical compound is a GABA_{B}R1/R2 receptor agonist which comprises contacting cells with the compound under conditions permitting the activation of the GABA_{B}R1/R2 receptor, and detecting an increase in GABA_{B}R1/R2 receptor activity, so as to thereby determine whether the compound is a GABA_{B}R1/R2 receptor agonist.

This invention is directed to a process for determining whether a chemical compound is a GABA_{B}R1/R2 receptor antagonist which comprises contacting cells containing nucleic acid encoding and expressing on their cell surface the GABA_{B}R1/R2 receptor, wherein such cells do not normally express the GABA_{B}R1/R2 receptor, with the compound in the presence of a known GABA_{B}R1/R2 receptor agonist, under conditions permitting the activation of the GABA_{B}R1/R2 receptor, and detecting a decrease in GABA_{B}R1/R2 receptor activity, so as to thereby determine whether the compound is a GABA_{B}R1/R2 receptor antagonist.

This invention is directed to a process for determining whether a chemical compound activates a GABA_{B}R1/R2 receptor, which comprises contacting cells producing a second messenger response and expressing on their cell surface the GABA_{B}R1/R2 receptor, wherein such cells do not normally express the GABA_{B}R1/R2 receptor, with the chemical compound under conditions suitable for activation of the GABA_{B}R1/R2 receptor, and measuring the second messenger response in the presence and in the absence of the chemical compound, a change in the second messenger response in the presence of the chemical compound indicating that the compound activates the GABA_{B}R1/R2 receptor.

This invention is directed to a process for determining whether a chemical compound inhibits activation of a GABA_{B}R1/R2 receptor, which comprises separately contacting cells producing a second messenger response and expressing on their cell surface the GABA_{B}R1/R2 receptor, wherein such cells do not normally express the GABA_{B}R1/R2 receptor, with both the chemical compound and a second chemical compound known to activate the GABA_{B}R1/R2 receptor, and with only the second chemical compound, under conditions suitable for activation of the GABA_{B}R1/R2 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the GABA_{B}R1/R2 receptor.

This invention is directed to a method of screening a plurality of chemical compounds not known to activate a GABA_{B}R1/R2 receptor to identify a compound which activates the GABA_{B}R1/R2 receptor which comprises:

(a) contacting cells containing nucleic acid encoding and expressing on their cell surface the GABA_{B}R1/R2 receptor, wherein such cells do not normally express the GABA_{B}R1/R2 receptor, with the plurality of compounds not known to activate the GABA_{B}R1/R2 receptor, under conditions permitting activation of the GABA_{B}R1/R2 receptor;

(b) determining whether the activity of the GABA_{B}R1/R2 receptor is increased in the presence of the compounds, and if it is increased;

(c) separately determining whether the activation of the GABA_{B}R1/R2 receptor is increased by each compound included in the plurality of compounds, so as to thereby identify the compound or compounds present in such plurality of compounds which activates the GABA_{B}R1/R2 receptor.

This invention is directed to a method of screening a plurality of chemical compounds not known to inhibit the activation of a GABA_{B}R1/R2 receptor to identify a compound which inhibits the activation of the GABA_{B}R1/R2 receptor, which comprises:

(a) contacting cells containing nucleic acid encoding and expressing on their cell surface the GABA_{B}R1/R2 receptor, wherein such cells do not normally express the GABA_{B}R1/R2 receptor, with the plurality of compounds in the presence of a known GABA_{B}R1/R2 receptor agonist, under conditions permitting activation of the GABA_{B}R1/R2 receptor;

(b) determining whether the activation of the GABA_{B}R1/R2 receptor is reduced in the presence of the plurality of compounds, relative to the activation of the GABA_{B}R1/R2 receptor in the absence of the plurality of compounds, and if it is reduced;

(c) separately determining the inhibition of activation of the GABA_{B}R1/R2 receptor for each compound included in the plurality of compounds, so as to thereby identify the compound or compounds present in such a plurality of compounds which inhibits the activation of the GABA_{B}R1/R2 receptor.

This invention is directed to a process for determining whether a chemical compound is a GABA_{B}R1/R2 receptor agonist, which comprises preparing a membrane fraction from cells which comprise nucleic acid encoding and expressing on their cell surface the GABA_{B}R1/R2 receptor, wherein such cells do not normally express the GABA_{B}R1/R2 receptor, separately contacting the membrane fraction with both the chemical compound and GTPΥS, and with only GTPΥS, under conditions permitting the activation of the GABA_{B}R1/R2 receptor, and detecting GTPΥS binding to the membrane fraction, an increase in GTPΥS binding in the presence of the compound indicating that the chemical compound activates the GABA_{B}R1/R2 receptor.

This invention is directed to a process for determining whether a chemical compound is a GABA_{B}R1/R2 receptor antagonist, which comprises preparing a membrane fraction from cells which comprise nucleic acid encoding and expressing on their cell surface the GABA_{B}R1/R2 receptor, wherein such cells do not normally express the GABA_{B}R1/R2 receptor, separately contacting the membrane fraction with the chemical compound, GTPΥS and a second chemical compound known to activate the GABA_{B}R1/R2 receptor, with GTPΥS and only the second compound, and with GTPΥS alone, under conditions permitting the activation of the GABA_{B}R1/R2 receptor, detecting GTPΥS binding to each membrane fraction, and comparing the increase in GTPΥS binding in the presence of the compound and the second compound relative to the binding of GTPΥS alone, to the increase in GTPΥS binding in the presence of the second chemical compound known to activate the GABA_{B}R1/R2 receptor relative to the binding of GTPΥS alone, a smaller increase in GTPΥS binding in the presence of the compound and the second compound indicating that the compound is a GABA_{B}R1/R2 receptor antagonist.

This invention is directed to a method of treating spasticity in a subject which comprises administering to the subject an amount of a compound which is an agonist of a GABA_{B}R1/R2 receptor effective to treat spasticity in the subject.

This invention is directed to a method of treating asthma in a subject which comprises administering to the subject an amount of a compound which is a GABA_{B}R1/R2 receptor agonist effective to treat asthma in the subject.

This invention is directed to a method of treating incontinence in a subject which comprises administering to the subject an amount of a compound which is a GABA_{B}R1/R2 receptor agonist effective to treat incontinence in the subject.

This invention is directed to a method of decreasing nociception in a subject which comprises administering to the subject an amount of a compound which is a GABA_{B}R1/R2 receptor agonist effective to decrease nociception in the subject.

This invention is directed to a use of a GABA_{B}R2 agonist as an antitussive agent which comprises administering to the subject an amount of a compound which is a GABA_{B}R1/R2 receptor agonist effective as an antitussive agent in the subject.

This invention is directed to a method of treating drug addiction in a subject which comprises administering to the subject an amount of a compound which is a GABA_{B}R1/R2 receptor agonist effective to treat drug addiction in the subject.

This invention is directed to a method of treating Alzheimer's disease in a subject which comprises administering to the subject an amount of a compound which is a GABA_{B}R1/R2 receptor antagonist effective to treat Alzheimer's disease in the subject.

This invention is directed to a peptide selected from the group consisting of:

| | |
|---|---|
| a) P L Y S I L S A L T I L G M I M A S A F L F F N I | (SEQ ID NO:48) |
| K N; | |
| b) L I I L G G M L S Y A S I F L F G L D G S F V S E | (SEQ ID NO:49) |
| K T; | |
| c) C T V R T W I L T V G Y T T A F G A M F A K T W R; | (SEQ ID NO:50) |
| d) Q K L L V I V G G M L L I D L C I L I C W Q; | (SEQ ID NO:51) |
| e) M T I W L G I V Y A Y K G L L M L F G C F L A W; | (SEQ ID NO:52) |
| f) A L N D S K Y I G M S V Y N V C I M C I I G A A V; | (SEQ ID NO:53) and |
| g) C I V A L V I I F C S T I T L C L V F V P K L I T | |
| L R T N. | |

This invention is directed to a compound that prevents the formation of a GABA_{B}R1/R2 receptor complex.

Finally, this invention provides a process for making a composition of matter which specifically binds to a GABA_{B}R1/R2 receptor which comprises identifying a chemical compound using any of the processes described herein for identifying a compound which binds to and/or activates or inhibits activation of a GABA_{B}R1/R2 receptor and then synthesizing the chemical compound or a novel structural and functional analog or homolog thereof. This invention further provides a process for preparing a pharmaceutical composition which comprises admixing a pharmaceutically acceptable carrier and a pharmaceutically acceptable amount of a chemical compound identified by any of the processes described herein for identifying a compound which binds to and/or activates or inhibits activation of a GABA_{B}R1/R2 receptor or a novel structural and functional analog or homolog thereof.

### BRIEF DESCRIPTION OF THE FIGURES

FIGS. 1A-1E Nucleotide coding sequence of the human GABA_{B}R2 polypeptide (SEQ ID NO:1), with partial 5′ and 3′ untranslated sequences. Two possible start (ATG) codons are underlined as well as the stop codon (TAA).

FIGS. 2A-2D Deduced amino acid sequence of the human GABA_{B}R2 polypeptide (SEQ ID NO:2) encoded by the nucleotide sequence shown in FIGS. 1A-1E.

FIGS. 3A-3D Nucleotide coding sequence of the rat GABA_{B}R2 polypeptide (SEQ ID NO:3). Start (ATG) and stop (TAG) codons are underlined.

FIGS. 4A-4D Deduced amino acid sequence of the rat GABA_{B}R2 polypeptide (SEQ ID NO:4) encoded by the nucleotide sequence shown in FIGS. 3A-3D.

FIGS. 5A-5D Amino acid sequence of the human GABA_{B}R2 polypeptide (SEQ ID NO:2) with brackets above the sequence showing the boundaries of seven (7) putative transmembrane domains, numbered consecutively from I to VII.

FIGS. 6A-6B. Measurement of EC₅₀ for GABA in a cumulative concentration response assay in oocytes expressing GABA_{B}R1b/GABA_{B}R2 +GIRKs. FIG. 6A: Electrophysiological trace from a voltage clamped oocyte showing increasing inward currents evoked successively by concentrations of GABA ranging from 0.03 to 30 μM. Numbers over bars indicate concentration of GABA in μM. hK is 49 mM external K⁺. FIG. 6B: Averaged responses from 3-6 oocytes plotted vs. concentration of GABA results in an EC₅₀ value of 1.76 μM. For each oocyte, currents were normalized to the maximum response at 30 μM.

FIG. 7. Concentration response relationship for baclofen in oocytes expressing GABA_{B}R1b/GABA_{B}R2+GIRKs. Methods are as described for FIG. 6.

FIG. 8. Current voltage relationship for the current activated by GABA in oocytes expressing GABA_{B}R1b/GABA_{B}R2+GIRKs. Voltage ramps (50 mv/s) from −140 to +40 mV were applied in the presence of GABA (in hK) and again in the presence of GABA+100 μM Ba⁺⁺ to block inward rectifier current. The resulting traces were subtracted (GABA alone−GABA+Ba⁺⁺) to yield the Ba⁺⁺-sensitive portion of the GABA-stimulated current. As expected for GIRK current, the current displays steep inward rectification and reverses near the predicted equilibrium potential for K+(−23 mV in hK).

FIGS. 9A-9B. Electrophysiological responses under voltage clamp conditions to GABA in an HEK-293 cell transiently transfected with GABA_{B}R1b/GABA_{B}R2+GIRKs. A) The continuous trace (in presence of 25 mM K⁺) shows a small constitutive inward rectifier current that is blocked by Ba⁺⁺ (100 μM), and a much larger inward current induced by application of GABA that is also blocked by Ba⁺⁺. A second GABA-evoked current is abolished by the selective antagonist CGP55845. After a 1 minute wash period GABA-responsivity returns. B) Concentration response relation for GABA in 5 HEK-293 cells expressing GABA_{B}R1b/GABA_{B}R2+GIRKs. (See FIG. 6B for details.)

FIG. 10. Alignment of amino acids predicted for rat GABA_{B}R2 and rat GABA_{B}R1 (SEQ ID NO:55). Horizontal bars indicate TM regions.

FIGS. 11A-11D. Photomicrographs showing the regional distribution of the GABA_{B}R1 (A,C) and GABA_{B}R2 (B,D) mRNAs in representative coronal rat brain sections. Hypothalamus and caudate-putamen are identified with arrow heads and arrows, respectively (A,B). Arrows identify Purkinje cell layer in cerebellum (C,D).

FIGS. 12A-12B. High magnification micrographs of Purkinje cell layer from alternate serial sections showing co-localization of GABA_{B}R2 transcripts using digoxigenin-labeled probes (A) and GABA_{B}R1 transcripts using [³⁵S]dATP-labeled probes (B) in the same cells (asterisks). Scale bar=30 μM.

FIGS. 13A-13B. FIG. 13A: Response to GABA (100 μM) from oocyte expressing GABA_{B}R1, GABA_{B}R2, and GIRKs (lower trace). Similar oocyte pretreated 6 h earlier with pertussis toxin (2 ng injected; upper trace). FIG. 13B: Summary of mean response amplitudes from oocytes expressing various combinations of GABA_{B}R1 and GABA_{B}R2 plus GIRKs. Responses are to 100 μM GABA (solid bars) or 100 μM baclofen (open bar). Number of observations are in parenthesis.

FIGS. 14A-14B. FIG. 14A: Response to GABA or baclofen (100 μM in 25 mM K⁺) in HEK293 cells expressing GIRKs along with GABA_{B}R1b, GABA_{B}R2, or both. FIG. 14B: Summary of mean response amplitudes from HEK293 cells co-transfected with various combinations and ratios of cDNA. To prepare different ratios of GABA_{B}R1b:GABA_{B}R2 the most abundant cDNA was held constant at 0.6 μg/dish and the other cDNA was reduced by a factor of 10 or 100. Responses are to 100 μM GABA. Number of observations are shown in parenthesis. FIGS. 15A-15B. FIG. 15A: Agonist concentration-effect curves for 3-APMPA in oocytes (open triangle), GABA in oocytes (open circle) and HEK293 cells (solid circle), and baclofen in oocytes (open square). FIG. 15B: Right-ward shifts in the GABA concentration-response curve (solid circle) caused by CGP55845 at 50 nM (open triangle) and CGP54626 at 5 μM (open circle). Each point is the average response from 4-6 oocytes.

FIG. 16. Microphysiometric response to baclofen (100 μM) from CHO cells expressing combinations of GABA_{B}R1 and GABA_{B}R2 (n=4).

FIGS. 17A-17D. Co-localization of GABA_{B}R1 and GABA_{B}R2 in HEK293 cells by dual wavelength scanning confocal microscopy. FIG. 17A: Green channel showing GABA_{B}R1^{RGS6xH} (labeled with FITC) in cell expressing both GABA_{B}R1^{RGS6xH} and GABA_{B}R2^{HA}. FIG. 17B: Red channel showing GABA_{B}R2^{HA} (labeled with TRITC) localization in the same cell. FIG. 17C: Dual channel image of the same cell reveals a predominant yellow hue caused by the co-localization of fluorescent tags for GABA_{B}R1^{RGS6xH} and GABA_{B}R2^{HA}. FIG. 17D: Dual wavelength image of cell expressing GABA_{B}R2^{HA} (red) and NPY Y5^{Flag} (green). Note the low degree of spatial overlap of the two polypeptides.

FIGS. 18A-18C. Identification of GABA_{B}R1 and GABA_{B}R2 in cell lysates and immunoprecipitates. FIG. 18A: Detection of GABA_{B}R1^{RGS6xH} in whole cell extracts from cells expressing either or both polypeptides. Proteins labeled with anti-His or anti-HA, migrate as monomeric and dimeric forms. FIG. 18B: Detection of GABA_{B}R2^{HA} in whole cell extracts from cells expressing either or both. Labels over lanes denote which polypeptides were transfected. Proteins labeled with anti-His or anti-HA, migrate as monomeric and dimeric forms. FIG. 18C: Co-immunoprecipitation of GABA_{B}R1^{RGS6xH} and GABA_{B}R2^{HA}. Variously transfected cells were immunoprecipitated (IP) with anti-HA or anti-His antibodies, subjected to SDS-PAGE, blotted, and probed for the presence of the HA epitope. Note that in anti-His immunoprecipitated material, HA immunoreactivity appears only in the lane from cells expressing both proteins.

FIG. 19. Rostro-caudal distribution of the GABA_{B}R2 mRNA in coronal rat brain sections(A-F)and spinal cord (G). Brightfield photomicrographs of the dorsal root (H) and trigeminal (I) ganglia showing silver grains over the cells indicating the presence of GABA_{B}R2 mRNA.

FIG. 20. (A) Detection of Na+/K+ ATPase by anti-alpha 1 subunit antibodies in membrane fractions enriched in (P1+) or depleted of (P2) plasma membranes (50 :g protein/lane). (B) Co-immunoprecipitation of GABA_{B}R1^{RGS6xH} and GABA_{B}R2^{HA} from solubilized P1+ membrane fractions. Note that in anti-His immunoprecipitated material, HA immunoreactivity appears only in the lane from cells expressing both proteins. (C) Western blot showing enrichment of GABA_{B}R2^{HA} in P1+ membrane fraction as compared to the P2 fraction.

FIG. 21. Photomicrographs showing the regional distribution of GABA_{B}R2 (A,C) and GABA_{B}R1b (B,D) mRNAs in pairs of adjacent coronal rat brain sections. Arrow heads identify Purkinje cell layer in cerebellum (A,B). High magnification views of hippocampal CA3 region showing both transcripts in cells from alternate sections (C,D). Arrows mark individual cells. Hybridization of GABA_{B}R2 (E) and GABA_{B}R1b (F) transcripts in large cells of mesencephalic trigeminal nucleus.

FIG. 22A-22D Nucleotide coding sequence of the human GABA_{B}R2 polypeptide (SEQ ID NO:46). Start (ATG) and stop (TAA) codons are underlined.

FIG. 23A-23D Deduced amino acid sequence of the human GABA_{B}R2 polypeptide (SEQ ID No:47) encoded by the nucleotide sequence shown in FIGS. 22A-22D.

FIG. 24 Acidification responses from CHO cells expressing GABA_{B}R1b/GABA_{B}R2 from human and rat.

FIG. 25 FLIPR responses to increasing concentrations of the GABA_{B} agonist 3-APMPA from individual wells of COS-7 cells transfected with GABA_{B}R1, GABA_{B}R2, and Gα_{q/13}.

FIG. 26 The mean of three individual concentration-effect curves to 3-APMPA.

### DETAILED DESCRIPTION OF THE INVENTION

In this application, the following standard abbreviations are used to indicate specific nucleotide bases:

| | |
|---|---|
| C = cytosine | A = adenine |
| T = thymine | G = guanine |

In this application, the term 7-TM spanning protein or a 7-TM protein indicates a protein presumed to have seven transmembrane regions which cross the cellular membrane band on its amino acid sequence.

In this application, the mammalian GABA_{B}R1/R2 receptor is understood to include rat-rat polypeptides (i.e. rat GABA_{B}R1 and rat GABA_{B}R2 polypeptides), rat-human polypeptides (i.e. either rat GABA_{B}R1 and human GABA_{B}R2 polypeptides or human GABA_{B}R1 and rat GABA_{B}R2 polypeptides), or human-human polypeptides (i.e. human GABA_{B}R1 and human GABA_{B}R2 polypeptides).

This invention is directed to an isolated nucleic acid encoding a GABA_{B}R2 polypeptide.

In one embodiment, the nucleic acid is DNA. In another embodiment, the DNA is cDNA. In another embodiment, the DNA is genomic DNA. In another embodiment, the nucleic acid is RNA. In another embodiment, the nucleic acid encodes a mammalian GABA_{B}R2 polypeptide. In another embodiment, the nucleic acid encodes a rat GABA_{B}R2 polypeptide. In another embodiment, the nucleic acid encodes a human GABA_{B}R2 polypeptide.

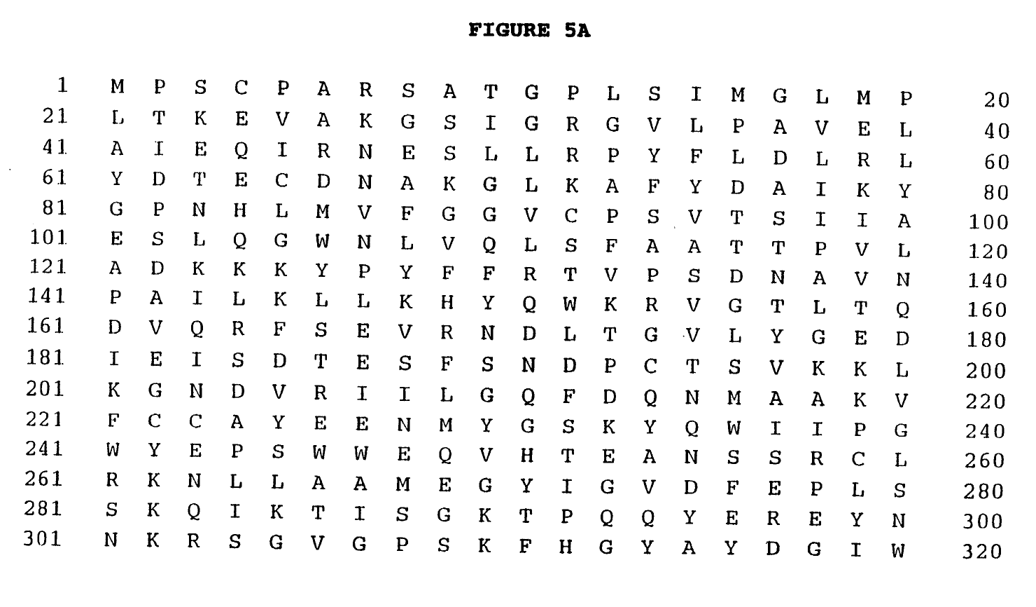

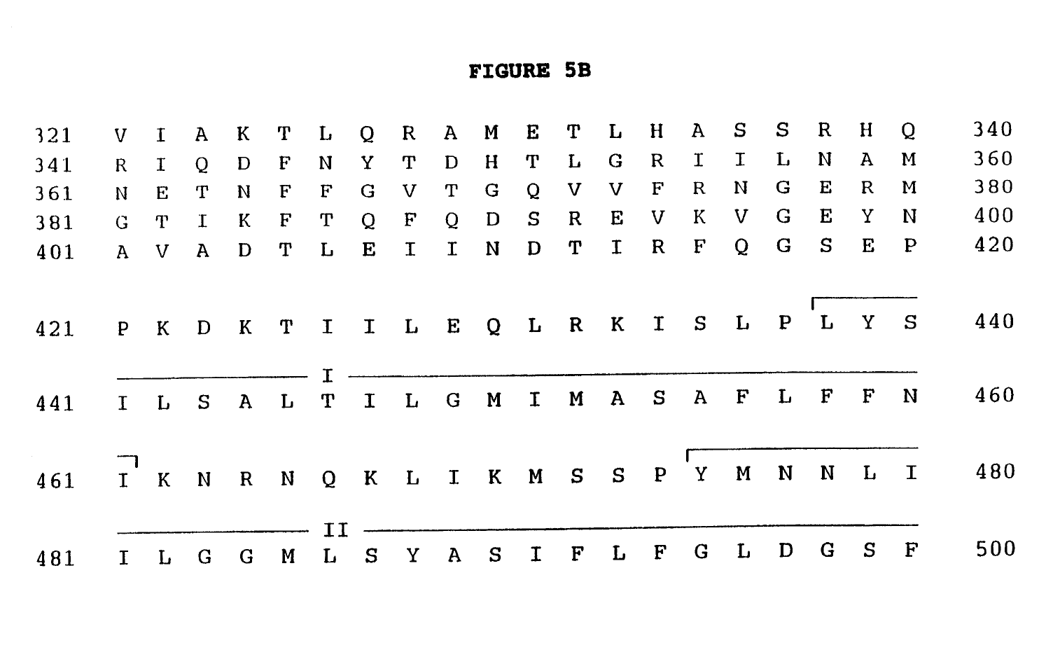

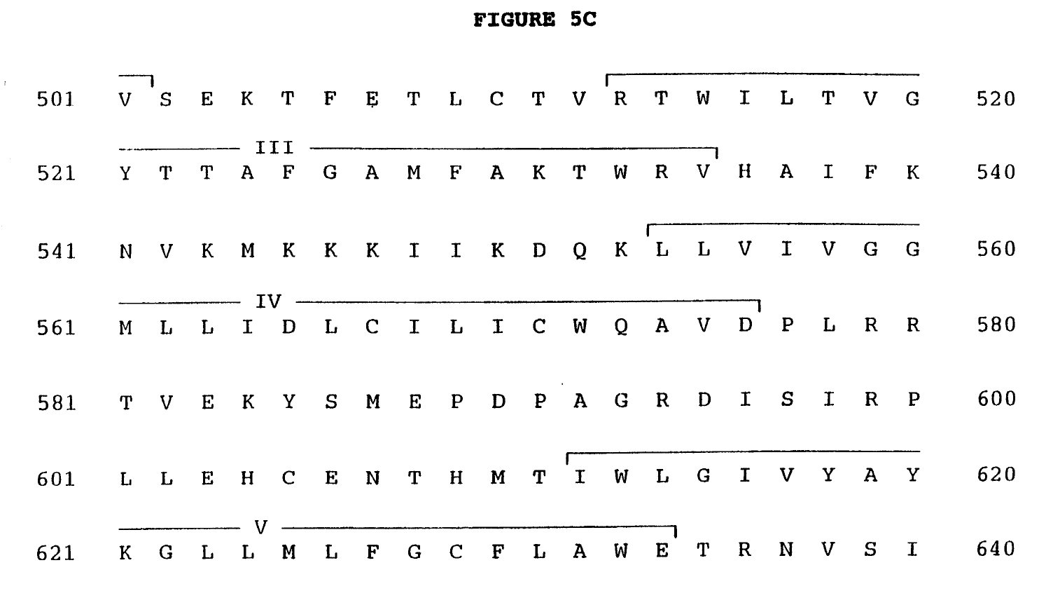

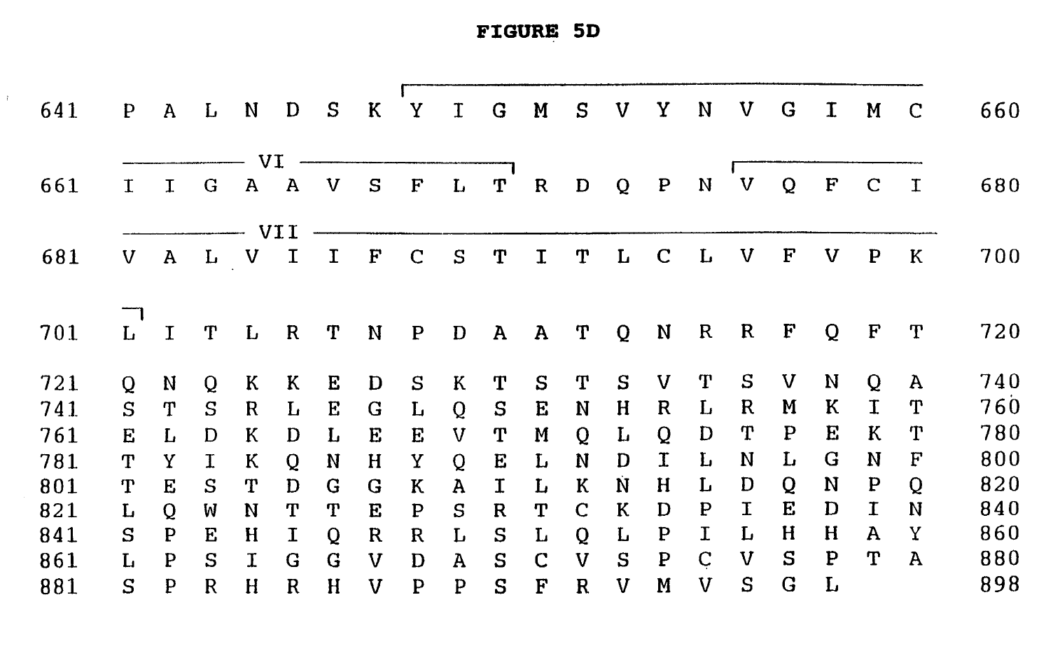

In another embodiment, the nucleic acid encodes a polypeptide characterized by an amino acid sequence in the transmembrane regions which has an identity of 90% or higher to the amino acid sequence in the transmembrane regions of the human GABA_{B}R2 polypeptide shown in FIGS. 5A-5D.

In another embodiment, the nucleic acid encodes a mammalian GABA_{B}R2 polypeptide which has substantially the same amino acid sequence as does the GABA_{B}R2 polypeptide encoded by the plasmid BO-55 (ATCC Accession No. 209104). In another embodiment, the nucleic acid encodes a rat GABA_{B}R2 polypeptide which has an amino acid sequence encoded by the plasmid BO-55 (ATCC Accession No. 209104).

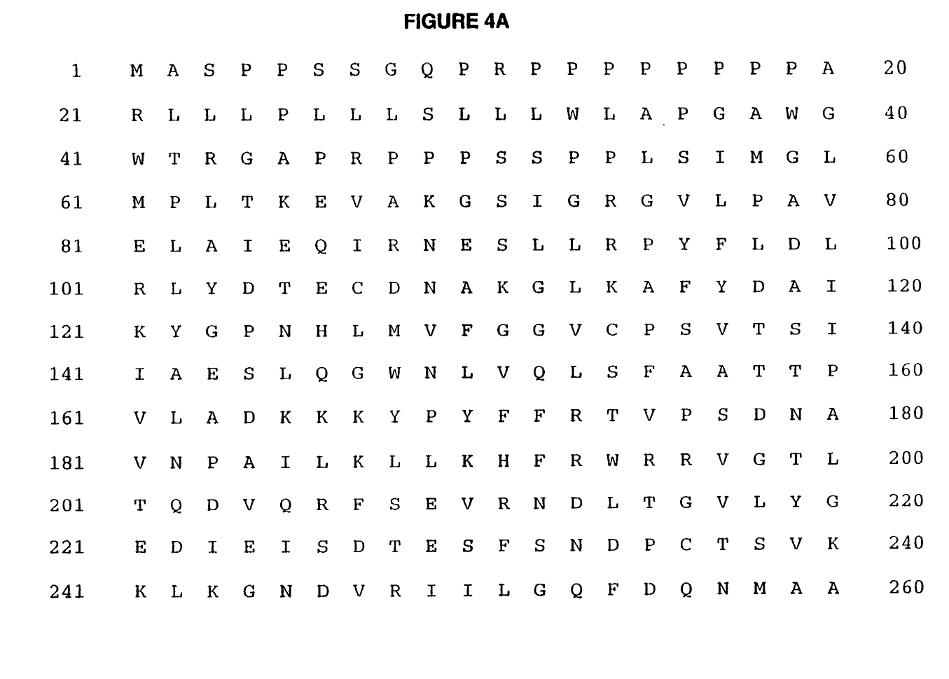

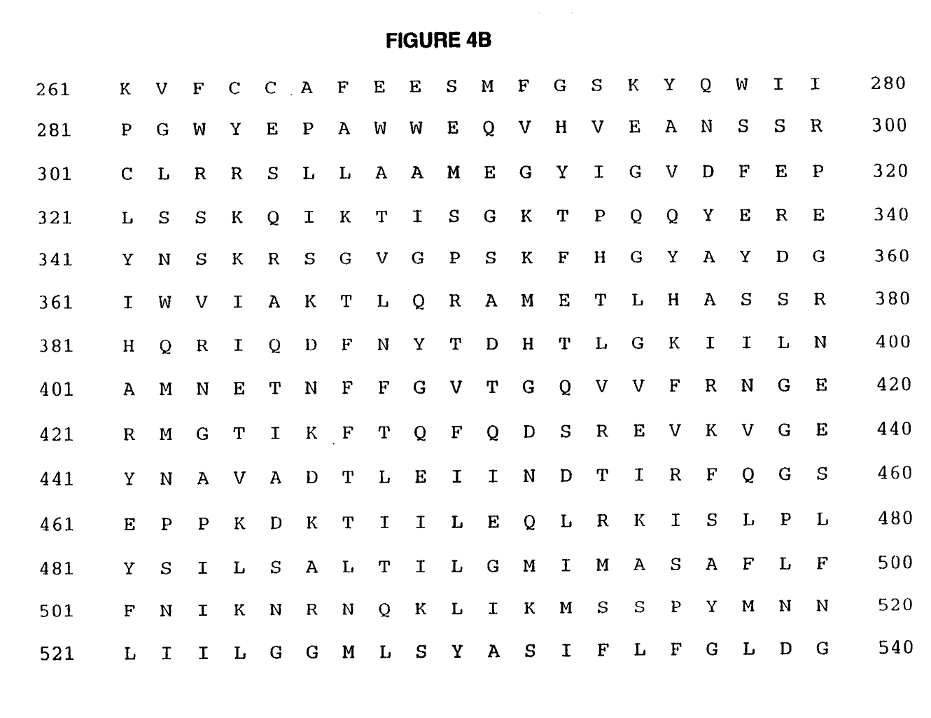

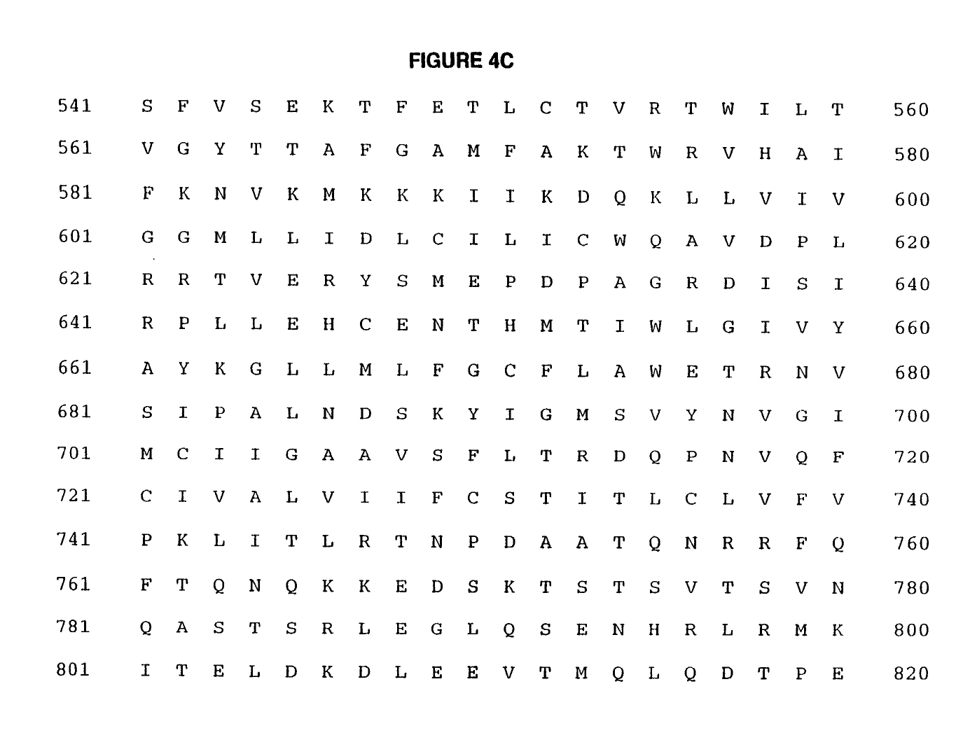

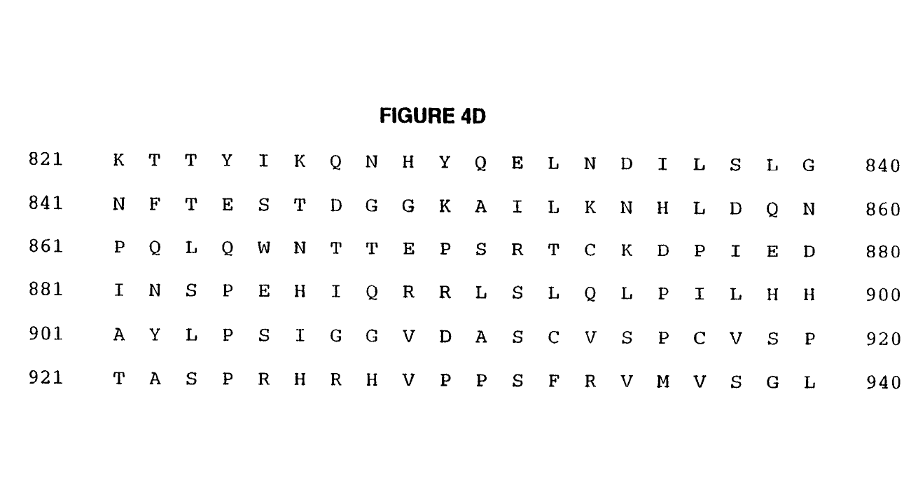

In another embodiment, the nucleic acid encodes a rat GABA_{B}R2 polypeptide having substantially the same amino acid sequence as the amino acid sequence shown in FIGS. 4A-4D (SEQ ID NO:4). In another embodiment, the nucleic acid encodes a rat GABA_{B}R2 polypeptide having the amino acid sequence shown in FIGS. 4A-4D (SEQ ID NO:4).

In another embodiment, the nucleic acid encodes a mammalian GABA_{B}R2 polypeptide which has substantially the same amino acid sequence as does the GABA_{B}R2 polypeptide encoded by the plasmid pEXJT3T7-hGABAB2 (ATCC Accession No. 203515). In another embodiment, the nucleic acid encodes a human GABA_{B}R2 polypeptide which has an amino acid sequence encoded by the plasmid pEXJT3T7-hGABAB2 (ATCC Accession No. 203515).

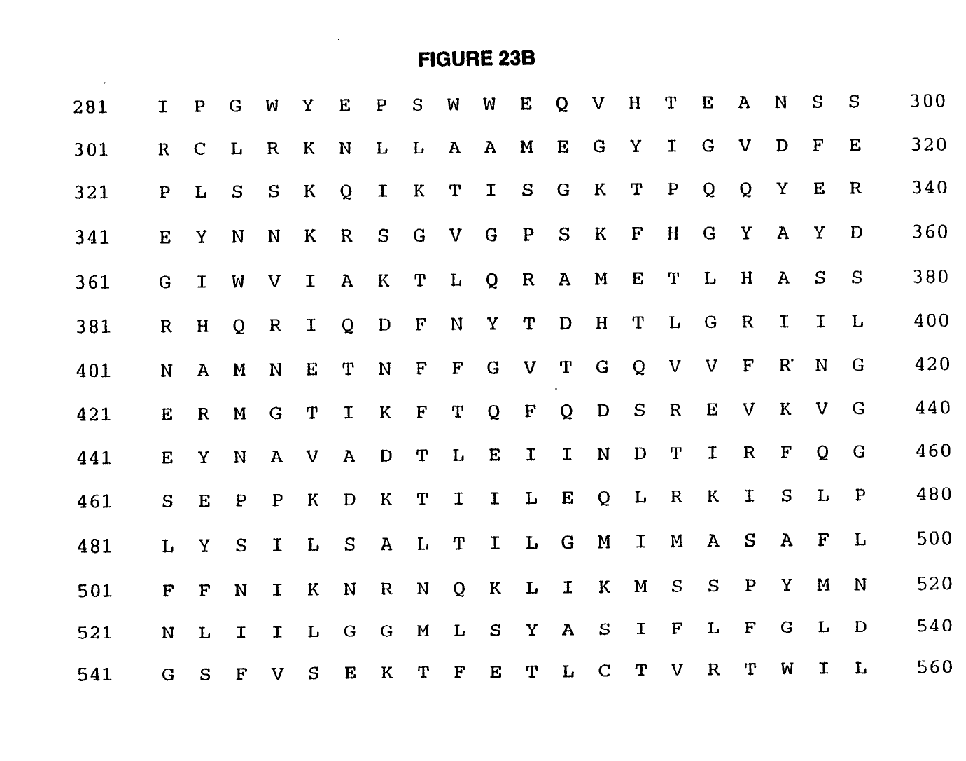

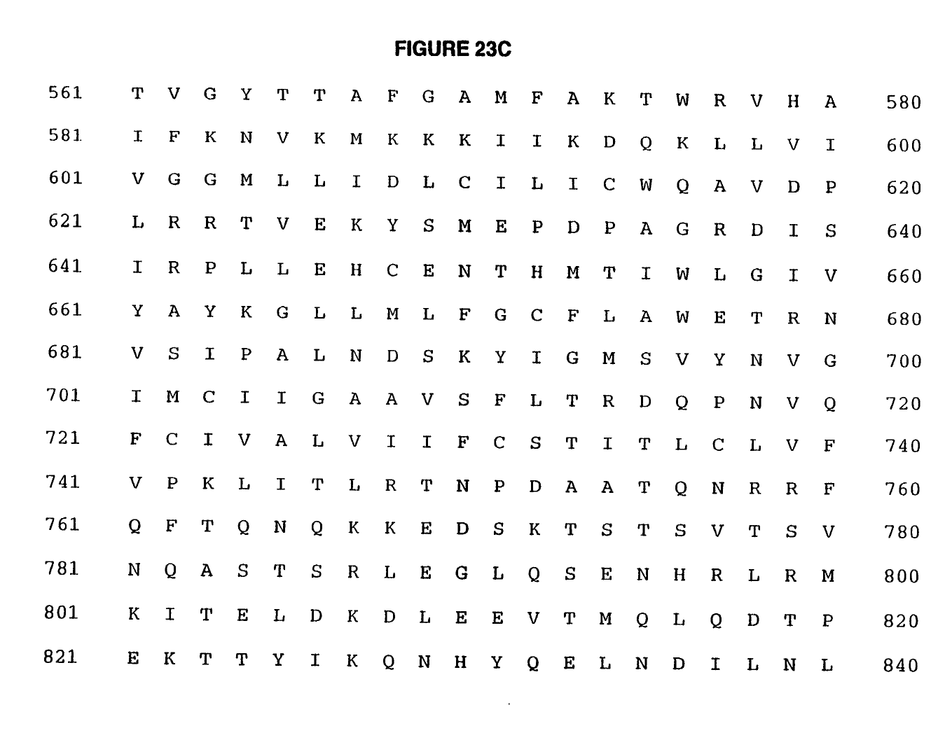

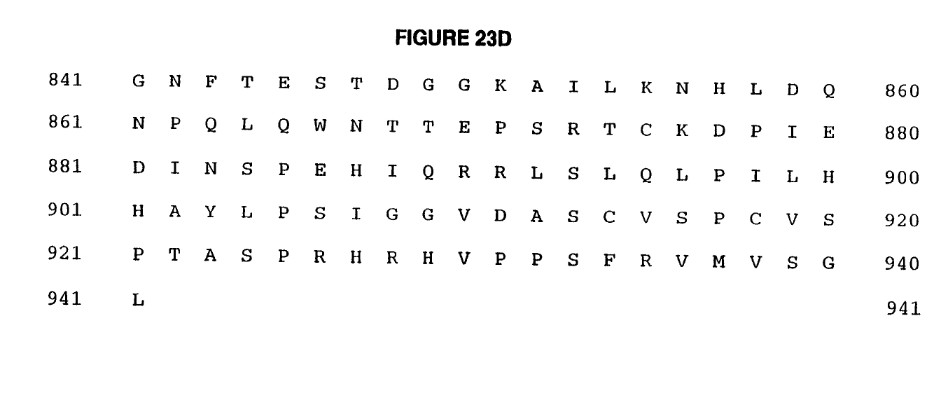

In another embodiment, the human GABA_{B}R2 polypeptide has a sequence, which sequence comprises substantially the same amino acid sequence as the sequence shown in FIGS. 23A-23D (SEQ ID NO:47).

In another embodiment, the human GABA_{B}R2 polypeptide has a sequence, which sequence comprises the sequence shown in FIGS. 23A-23D (SEQ ID NO:47).

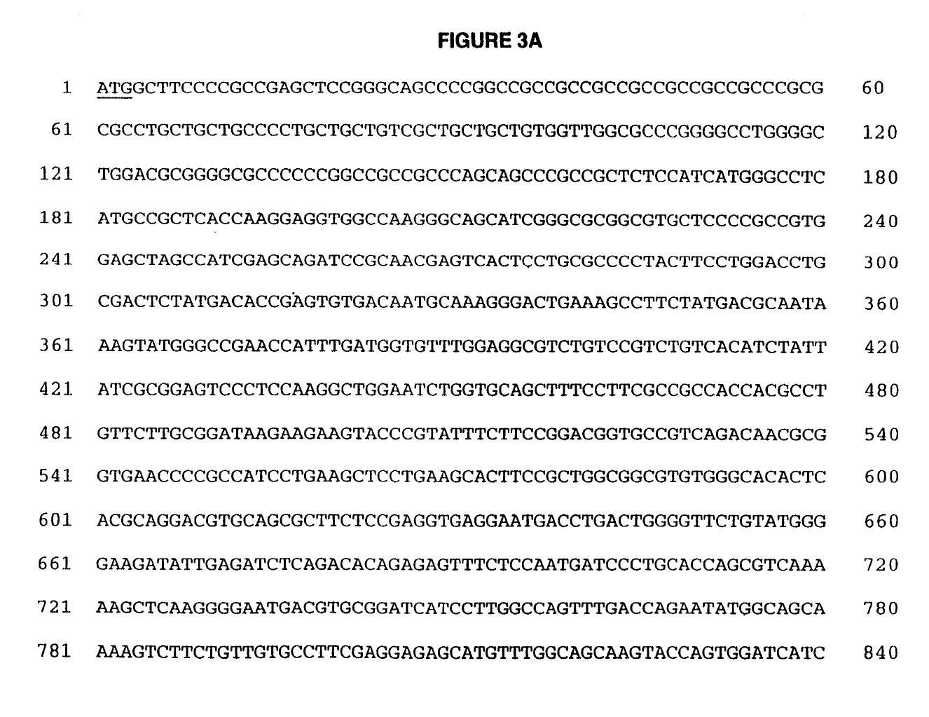

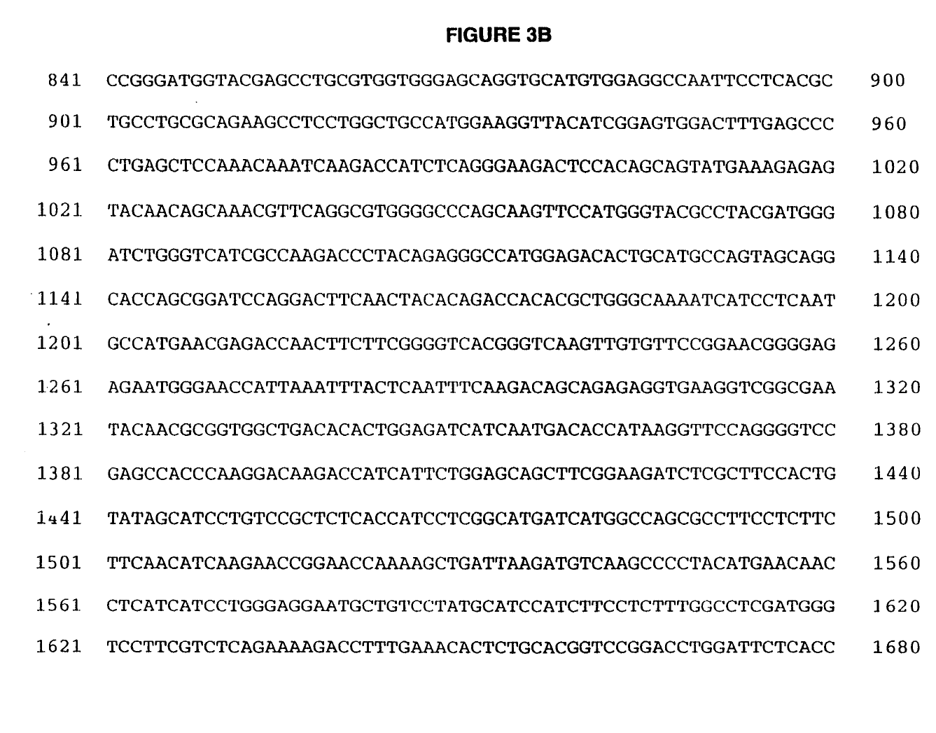

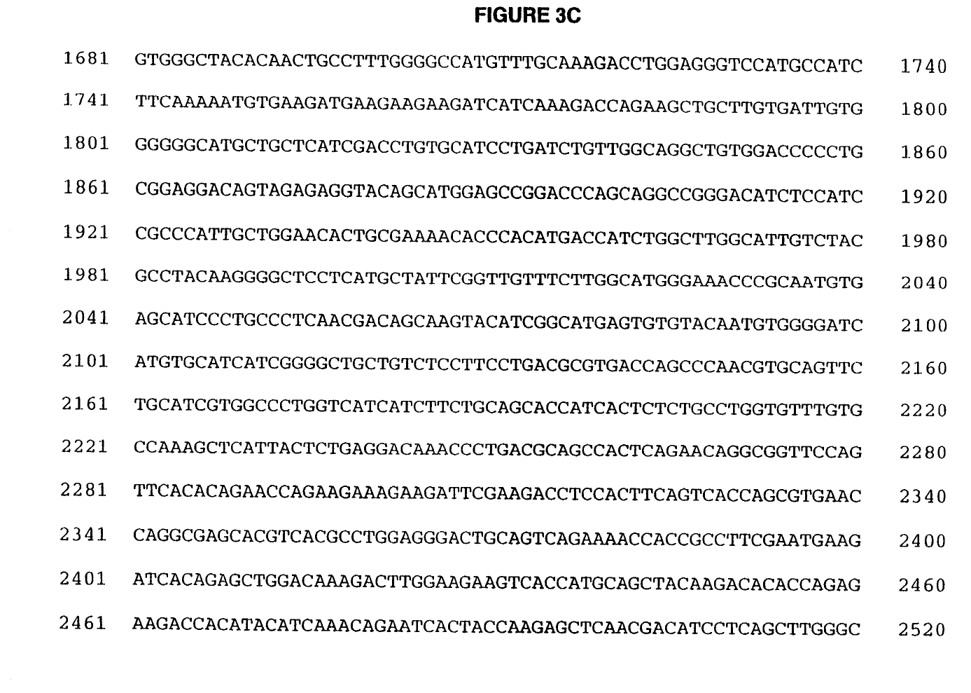

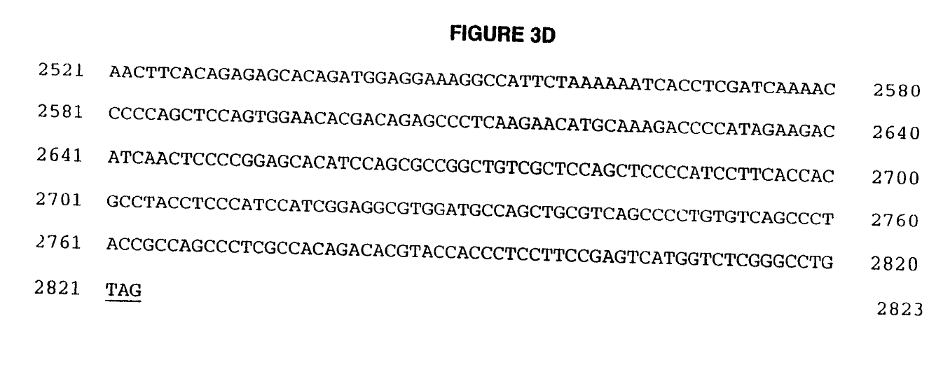

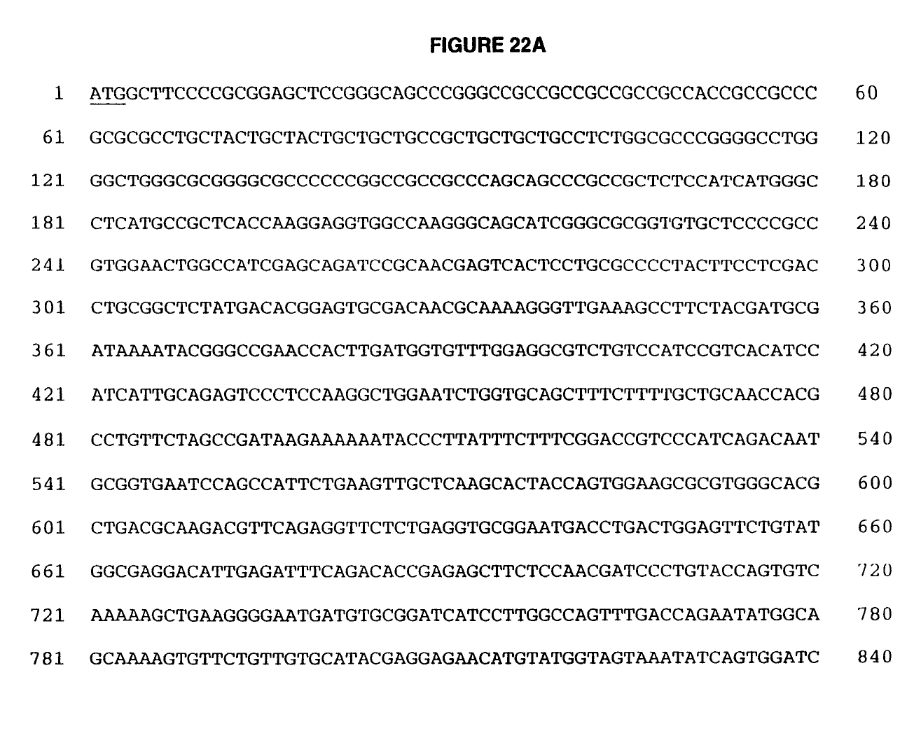

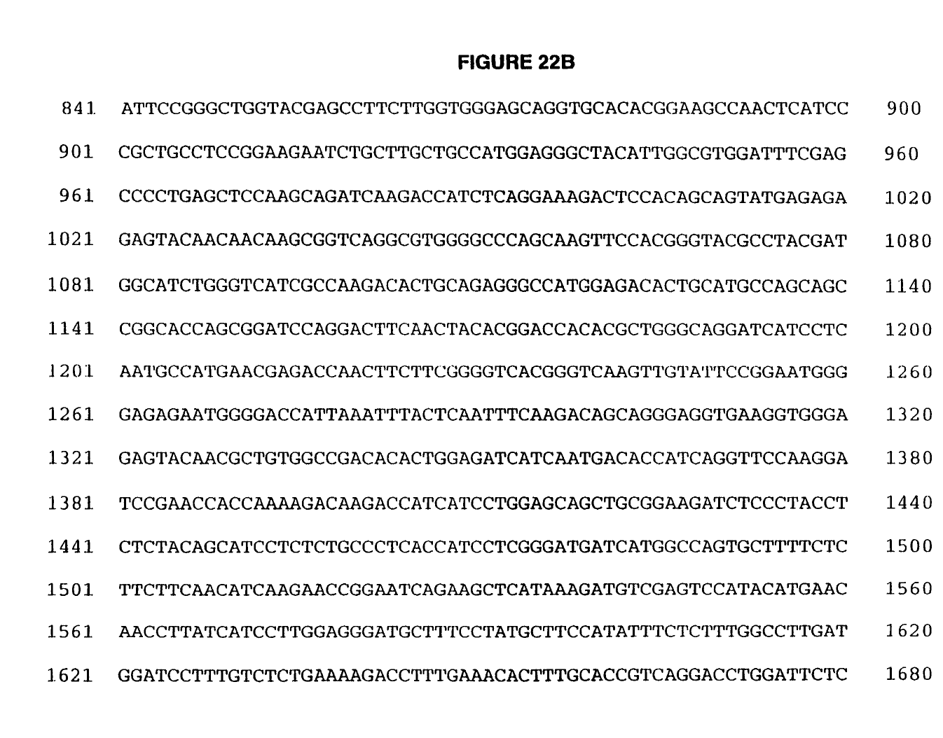

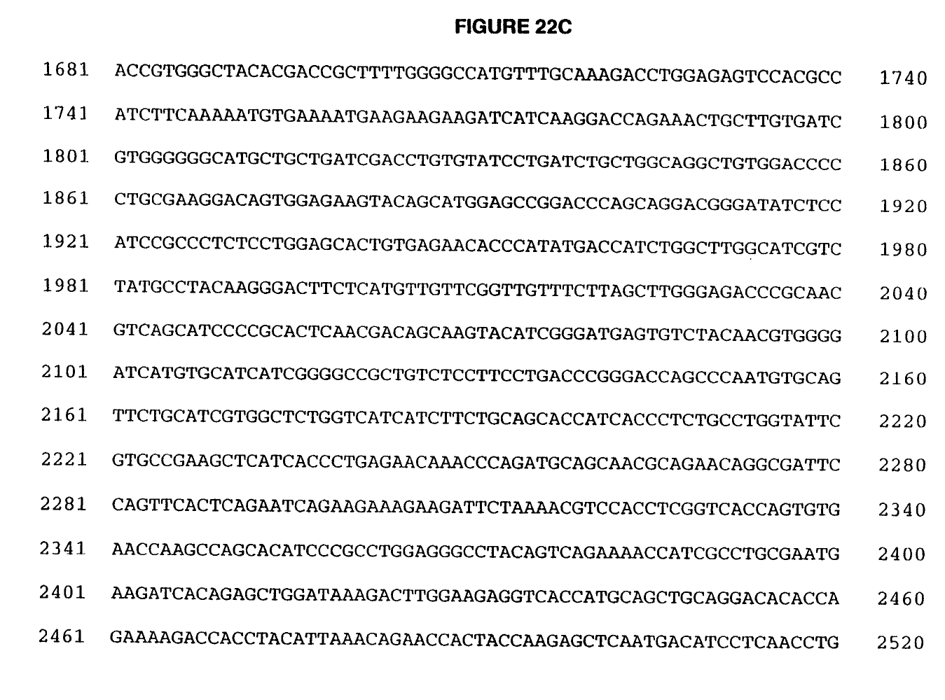

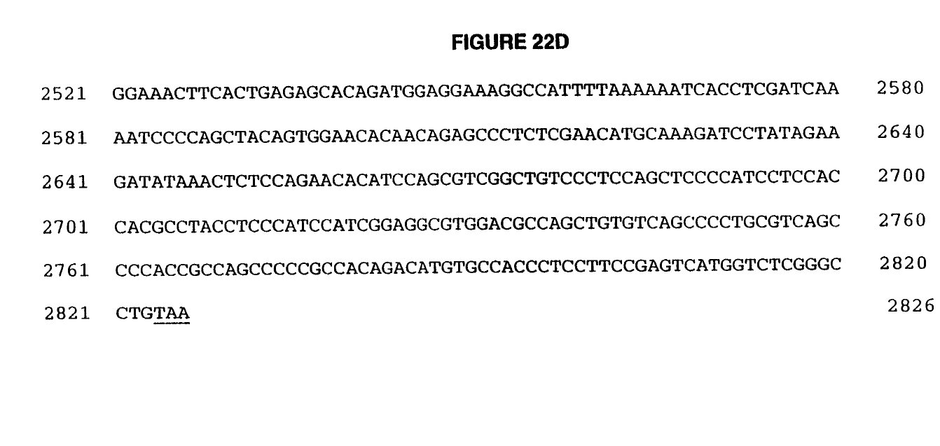

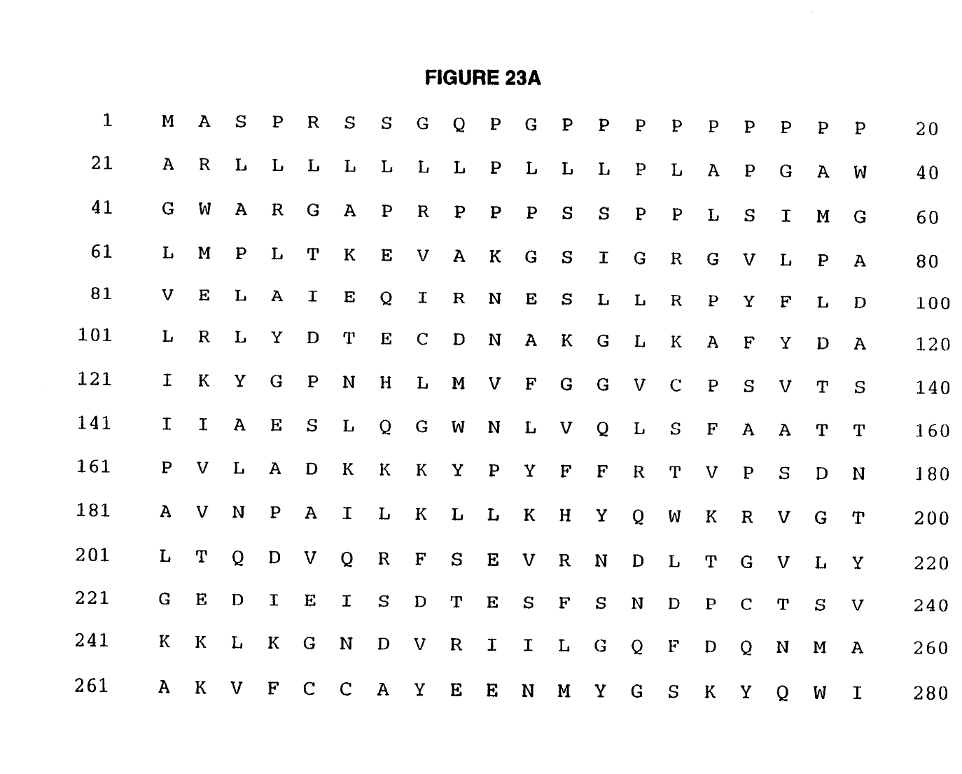

This application further supports an isolated nucleic acid encoding a GABA_{B}R2 polypeptide, the amino acid sequence of which is encoded by the nucleotide sequence set forth in either the FIGS. 22A-22D and 3A-3D.

Further, the human GABA_{B}R2 polypeptide described herein exhibits 38% amino acid identity with the GABA_{B}R1a polypeptide, while the rat GABA_{B}R2 polypeptide described herein exhibits 98% identity with the human GABA_{B}R2 polypeptide.

The ATG encoding the methionine at position 16 is surrounded by flanking sequences which correspond to the well-known Kozak consensus sequence for translation initiation (Kozak, 1989 and Kozak, 1991), thus the sequence from amino acid 16 through amino acid 898 is believed to be the most likely polypeptide expressed by the nucleic acid. Neither the ATG encoding methionine 1 nor the ATG encoding methionine 19 has the Kozak flanking sequences; however, it is to be understood that the present invention provides a GABA_{B}R2 polypeptide having any one of the three possible starting methionines.

This invention provides a splice variant of the polypeptides disclosed herein. This invention further provides for alternate translation initiation sites and alternately spliced or edited variants of nucleic acids encoding rat and human polypeptides of this invention.

Methods for production and manipulation of nucleic acid molecules are well known in the art.

This invention also encompasses DNAs and cDNAs which encode amino acid sequences which differ from those of the polypeptides of this invention, but which should not produce phenotypic changes. Alternatively, this invention also encompasses DNAS, cDNAs, and RNAs which hybridize to the DNA, cDNA, and RNA of the subject invention. Hybridization methods are well known to those of skill in the art.

The nucleic acids of the subject invention also include nucleic acid molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs where in one or more amino acid residues is added to a terminal or medial portion of the polypeptides) and which share some or all properties of naturally-occurring forms. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors.

The modified polypeptides of this invention may be transfected into cells either transiently or stably using methods well-known in the art, examples of which are disclosed herein.

This invention also provides for binding assays using the modified polypeptides, in which the polypeptide is expressed either transiently or in stable cell lines. This invention further provides for a compound identified using a modified polypeptide in a binding assay such as the binding assays described herein.

The nucleic acids described and claimed herein are useful for the information which they provide concerning the amino acid sequence of the polypeptide and as products for the large scale synthesis of the polypeptide by a variety of recombinant techniques. The nucleic acid molecule is useful for generating new cloning and expression vectors, transformed and transfected prokaryotic and eukaryotic host cells, and new and useful methods for cultured growth of such host cells capable of expression of the polypeptide and related products.

Vectors which comprise the isolated nucleic acid molecule described hereinabove also are provided. Suitable vectors comprise, but are not limited to, a plasmid or a virus. These vectors may be transformed into a suitable host cell to form a host cell expression system for the production of a GABA_{B}R2 polypeptide. Suitable host cells include, for example, neuronal cells such as the glial cell line C6, a Xenopus cell such as an oocyte or melanophore cell, as well as numerous mammalian cells and non-neuronal cells.

This invention further provides for any vector or plasmid which comprises modified untranslated sequences, which are beneficial for expression in desired host cells or for use in binding or functional assays. For example, a vector or plasmid with untranslated sequences of varying lengths may express differing amounts of the polypeptide depending upon the host cell used. In an embodiment, the vector or plasmid comprises the coding sequence of the polypeptide and the regulatory elements necessary for expression in the host cell.

As used herein, the phrase “specifically hybridizing” means the ability of a nucleic acid molecule to recognize a nucleic acid sequence complementary to its own and to form double-helical segments through hydrogen bonding between complementary base pairs. The term “complementary” is used in its usual sense in the art, i.e., G and C are complementary and A is complementary to T (or U in RNA), such that two strands of nucleic acid are “complementary” only if every base matches the opposing base exactly.

This invention is directed to a purified GABA_{B}R2 protein.

This invention is directed to a vector comprising a above-identified nucleic acid.

In one embodiment, the vector is adapted for expression in a bacterial cell which comprises the regulatory elements necessary for expression of the nucleic acid in the bacterial cell operatively linked to the nucleic acid encoding a GABA_{B}R2 polypeptide so as to permit expression thereof.

In another embodiment, the vector is adapted for expression in an amphibian cell which comprises the regulatory elements necessary for expression of the nucleic acid in the amphibian cell operatively linked to the nucleic acid encoding a GABA_{B}R2 polypeptide so as to permit expression thereof.

In another embodiment, the vector is adapted for expression in a yeast cell which comprises the regulatory elements necessary for expression of the nucleic acid in the yeast cell operatively linked to the nucleic acid encoding a GABA_{B}R2 polypeptide so as to permit expression thereof.

In another embodiment, the vector is adapted for expression in an insect cell which comprises the regulatory elements necessary for expression of the nucleic acid in the insect cell operatively linked to the nucleic acid encoding the GABA_{B}R2 polypeptide so as to permit expression thereof.

In one embodiment, the vector is a baculovirus.

In another embodiment, the vector is adapted for expression in a mammalian cell which comprises the regulatory elements necessary for expression of the nucleic acid in the mammalian cell operatively linked to the nucleic acid encoding a GABA_{B}R2 polypeptide so as to permit expression thereof.

In one embodiment, the vector is a plasmid.

In a further embodiment, the plasmid is designated BO-55 (ATCC Accession No. 209104).

In a further embodiment, the plasmid is designated pEXJT3T7-hGABAB2 (ATCC Accession No. 203515).

This invention provides a plasmid designated pEXJT3T7-hGABAB2 (ATCC Accession No. 203515) which comprises the regulatory elements necessary for expression of DNA in a mammalian cell operatively linked to DNA encoding the human polypeptide so as to permit expression thereof.

This plasmid (pEXJT3T7-hGABAB2) was deposited on Dec. 9, 1998, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Accession No. 203515.

This invention provides a plasmid designated BO-55 (ATCC Accession No. 209104) which comprises the regulatory elements necessary for expression of DNA in a mammalian cell operatively linked to DNA encoding the rat polypeptide so as to permit expression thereof.

This plasmid (BO-55) was deposited on Jun. 10, 1997, with the American Type Culture Collection (ATCC), 12301 Parklawn Drive, Rockville, Md. 20852, U.S.A. under the provisions of the Budapest Treaty for the International Recognition of the Deposit of Microorganisms for the Purposes of Patent Procedure and was accorded ATCC Accession No. 209104.

Nucleic acid probe technology is well known to those skilled in the art who will readily appreciate that such probes may vary greatly in length and may be labeled with a detectable label, such as a radioisotope or fluorescent dye, to facilitate detection of the probe. DNA probe molecules may be produced by insertion of a DNA molecule which encodes the polypeptides of this invention into suitable vectors, such as plasmids or bacteriophages, followed by transforming into suitable bacterial host cells, replication in the transformed bacterial host cells and harvesting of the DNA probes, using methods well known in the art. Alternatively, probes may be generated chemically from DNA synthesizers.

RNA probes may be generated by inserting the DNA molecule which encodes the polypeptides of this invention downstream of a bacteriophage promoter such as T3, T7 or SP6. Large amounts of RNA probe may be produced by incubating the labeled nucleotides with the linearized fragment where it contains an upstream promoter in the presence of the appropriate RNA polymerase.

This invention is directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within one of the two strands of the nucleic acid encoding the GABA_{B}R2 polypeptide contained in plasmid BO-55, and detecting hybridization of the probe to the nucleic acid.

This invention is directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within (a) the nucleic acid sequence shown in FIGS. 22A-22D (SEQ ID NO:46) or (b) the reverse complement to the nucleic acid sequence shown in FIGS. 22A-22D (SEQ ID NO:46), and detecting hybridization of the probe to the nucleic acid.

This invention is directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within one of the two strands of the nucleic acid encoding the GABA_{B}R2 polypeptide contained in plasmid pEXJT3T7-hGABAB2 and detecting hybridization of the probe to the nucleic acid.

This invention is directed to a method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within (a) the nucleic acid sequence shown in FIGS. 3A-3D (SEQ ID NO:3) or (b) the reverse complement to the nucleic acid sequence shown in FIGS. 3A-3D (SEQ ID NO:3), and detecting hybridization of the probe to the nucleic acid.

In one embodiment, the nucleic acid is DNA.

In another embodiment, the nucleic acid is RNA.

In one embodiment, the probe comprises at least 15 nucleotides complementary to a unique segment of the sequence of the nucleic acid molecule encoding the GABA_{B}R2 polypeptide.

This invention is directed to a method of detecting a nucleic acid encoding a GABA_{B}R2 polypeptide, which comprises contacting the nucleic acid with a probe comprising a nucleic acid of at least 15 nucleotides which is complementary to the antisense sequence of a unique segment of the sequence of the nucleic acid encoding the GABA_{B}R2 polypeptide, and detecting hybridization of the probe to the nucleic acid.

This invention is directed to a method of inhibiting translation of mRNA encoding a GABA_{B}R2 polypeptide which comprises contacting such mRNA with an antisense oligonucleotide having a sequence capable of specifically hybridizing to the above-identified mRNA, so as to prevent translation of the mRNA.

This invention is directed to a method of inhibiting translation of mRNA encoding a GABA_{B}R2 polypeptide which comprises contacting such mRNA with an antisense oligonucleotide having a sequence capable of specifically hybridizing to the above-identified genomic DNA.

In one embodiment, the oligonucleotide comprises chemically modified nucleotides or nucleotide analogues.

In another embodiment, the isolated antibody is capable of binding to a GABA_{B}R2 polypeptide encoded by an above-identified nucleic acid.

In another embodiment, the GABA_{B}R2 polypeptide is a human GABA_{B}R2 polypeptide.

This invention is directed to an antibody capable of competitively inhibiting the binding of an above-identified antibody to a GABA_{B}R2 polypeptide.

In one embodiment, the antibody is a monoclonal antibody.

In one embodiment, the monoclonal antibody is directed to an epitope of a GABA_{B}R2 polypeptide present on the surface of a GABA_{B}R2 polypeptide expressing cell.

In another embodiment, the oligonucleotide is coupled to a substance which inactivates mRNA.

In another embodiment, the substance which inactivates mRNA is a ribozyme.

This invention is directed to a pharmaceutical composition which comprises an amount of an above-identified antibody effective to block binding of a ligand to the GABA_{B}R2 polypeptide and a pharmaceutically acceptable carrier.

As used herein, “pharmaceutically acceptable carriers” means any of the standard pharmaceutically acceptable carriers. Examples include, but are not limited to, phosphate buffered saline, physiological saline, water and emulsions, such as oil/water emulsions.

Animal model systems which elucidate the physiological and behavioral roles of the polypeptides of this invention are produced by creating transgenic animals in which the activity of the polypeptide is either increased or decreased, or the amino acid sequence of the expressed polypeptide is altered, by a variety of techniques. Examples of these techniques include, but are not limited to: 1) Insertion of normal or mutant versions of DNA encoding the polypeptide, by microinjection, electroporation, retroviral transfection or other means well known to those skilled in the art, into appropriate fertilized embryos in order to produce a transgenic animal or 2) Homologous recombination of mutant or normal, human or animal versions of these genes with the native gene locus in transgenic animals to alter the regulation of expression or the structure of these polypeptide sequences. The technique of homologous recombination is well known in the art. It replaces the native gene with the inserted gene and so is useful for producing an animal that cannot express native polypeptides but does express, for example, an inserted mutant polypeptide, which has replaced the native polypeptide in the animal's genome by recombination, resulting in underexpression of the transporter. Microinjection adds genes to the genome, but does not remove them, and so is useful for producing an animal which expresses its own and added polypeptides, resulting in overexpression of the polypeptides.

One means available for producing a transgenic animal, with a mouse as an example, is as follows: Female mice are mated, and the resulting fertilized eggs are dissected out of their oviducts. The eggs are stored in an appropriate medium such as M2 medium. DNA or cDNA encoding a polypeptide of this invention is purified from a vector by methods well known in the art. Inducible promoters may be fused with the coding region of the DNA to provide an experimental means to regulate expression of the trans-gene. Alternatively, or in addition, tissue specific regulatory elements may be fused with the coding region to permit tissue-specific expression of the trans-gene. The DNA, in an appropriately buffered solution, is put into a microinjection needle (which may be made from capillary tubing using a pipet puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only method for inserting DNA into the egg cell, and is used here only for exemplary purposes.

This invention is directed to a transgenic, nonhuman mammal expressing DNA encoding a GABA_{B}R2 polypeptide.

This invention is directed to a transgenic, nonhuman mammal comprising a homologous recombination knockout of the native GABA_{B}R2 polypeptide.

This invention is further directed to a transgenic, nonhuman mammal whose genome comprises antisense DNA complementary to DNA encoding a GABA_{B}R2 polypeptide so placed as to be transcribed into antisense mRNA which is complementary to mRNA encoding such GABA_{B}R2 polypeptide and which hybridizes to such mRNA encoding such GABA_{B}R2 polypeptide, thereby reducing its translation.

This invention is directed to an above-identified transgenic, nonhuman mammal, wherein the DNA encoding the GABA_{B}R2 polypeptide additionally comprises an inducible promoter.

This invention is directed to an above-identified transgenic, nonhuman mammal, wherein the DNA encoding the GABA_{B}R2 polypeptide additionally comprises tissue specific regulatory elements.

This invention is directed to an above-identified transgenic, nonhuman mammal, wherein the transgenic, nonhuman mammal is a mouse.

This invention is directed to method of detecting the presence of a GABA_{B}R2 polypeptide on the surface of a cell which comprises contacting the cell with an above-identified antibody under conditions permitting binding of the antibody to the polypeptide, detecting the presence of the antibody bound to the cell, and thereby detecting the presence of a GABA_{B}R2 polypeptide on the surface of the cell.

This invention is directed to a method of preparing a purified GABA_{B}R2 polypeptide which comprises:

a. inducing cells to express a GABA_{B}R2 polypeptide;

b. recovering the polypeptide so expressed from the induced cells; and

c. purifying the polypeptide so recovered.

This invention is directed to a method of preparing the purified GABA_{B}R2 polypeptide which comprises:

a. inserting a nucleic acid encoding the GABA_{B}R2 polypeptide into a suitable vector;

b. introducing the resulting vector in a suitable host cell;

c. placing the resulting cell in suitable condition permitting the production of the GABA_{B}R2 polypeptide;

d. recovering the polypeptide produced by the resulting cell; and

e. isolating or purifying the polypeptide so recovered.

This invention is directed to a GABA_{B}R1/R2 receptor comprising two polypeptides, one of which is a GABA_{B}R2 polypeptide and another of which is a GABA_{B}R1 polypeptide.

This invention is directed to a method of forming a GABA_{B}R1/R2 receptor which comprises inducing cells to express both a GABA_{B}R1 polypeptide and a GABA_{B}R2 polypeptide.

GABA_{B}R1 as used in this application could be GABA_{B}R1a or GABA_{B}R1b. The observation that at least two variants of the GABA_{B}R1 polypeptide exist raises the possibility that GABA_{B}R2 splice variants may exist or that there may exist introns in coding or non-coding regions of the genes encoding the GABA_{B}R2 polypeptides. In addition, spliced form(s) of mRNA may encode additional amino acids either upstream of the currently defined starting methionine or within the coding region. Further, the existence and use of alternative exons is possible, whereby the mRNA may encode different amino acids within the region comprising the exon. In addition, single amino acid substitutions may arise via the mechanism of RNA editing such that the amino acid sequence of the expressed protein is different than that encoded by the original gene (Burns et al., 1996; Chu et al., 1996). Such variants may exhibit pharmacologic properties differing from the polypeptide encoded by the original gene.

The activity of a G-protein coupled receptor (GPCR) typically is measured using any of a variety of functional assays in which activation of the receptor in question results in an observable change in the level of some second messenger system, including but not limited to adenylate cyclase, calcium mobilization, arachidonic acid release, ion channel activity, inositol phospholipid hydrolysis or guanylyl cyclase. Heterologous expression systems utilizing appropriate host cells to express the nucleic acids of the subject invention are used to obtain the desired second messenger coupling. Receptor activity may also be assayed in an oocyte expression system.

The pharmacologic properties of the receptor described herein when GABA_{B}R2 is co-expressed with GABA_{B}R1, are similar to the pharmacologic properties of the GABA_{B} receptor observed using tissues. For convenience, in the context of the present invention applicants will refer to the product of the heterologous coexpression of GABA_{B}R2 and GABA_{B}R1 as the “GABA_{B}R1/R2 receptor.” Thus, a cell expressing nucleic acid encoding a GABA_{B}R1/R2 receptor is to be understood to refer to a cell expressing both nucleic acid encoding a GABA_{B}R1 polypeptide and nucleic acid encoding a GABA_{B}R2 polypeptide. In this application, GABA_{B}R1 can be either GABA_{B}R1a or GABA_{B}R1b.

This invention is directed to an antibody capable of binding to a GABA_{B}R1/R2 receptor, wherein the GABA_{B}R2 polypeptide is encoded by an above-identified nucleic acid.

This invention is directed to an above-identified antibody, wherein the GABA_{B}R2 polypeptide is a human GABA_{B}R2 polypeptide.

This invention is directed to an antibody capable of competitively inhibiting the binding of an above-identified antibody to a GABA_{B}R1/R2 receptor.

In one embodiment, the antibody is a monoclonal antibody.

This invention is directed to an above-identified monoclonal antibody directed to an epitope of a GABA_{B}R1/R2 receptor present on the surface of a GABA_{B}R1/R2 polypeptide expressing cell.

This invention is directed to a pharmaceutical composition which comprises an amount of an above-identified antibody effective to block binding of a ligand to the GABA_{B}R1/R2 receptor and a pharmaceutically acceptable carrier.

This invention is directed to a transgenic, nonhuman mammal expressing a GABA_{B}R1/R2 receptor, which is not naturally expressed by the mammal.

This invention is directed to a transgenic, nonhuman mammal comprising a homologous recombination knockout of the native GABA_{B}R1/R2 receptor.

In one embodiment, the transgenic nonhuman mammal is a mouse.

This invention is directed to a method of detecting the presence of a GABA_{B}R1/R2 receptor on the surface of a cell which comprises contacting the cell with an above-identified antibody under conditions permitting binding of the antibody to the receptor, detecting the presence of the antibody bound to the cell, and thereby detecting the presence of a GABA_{B}R1/R2 receptor on the surface of the cell.

This invention is directed to a method of determining the physiological effects of varying levels of activity of GABA_{B}R1/R2 receptors which comprises producing an above-identified transgenic nonhuman mammal whose levels of GABA_{B}R1/R2 receptor activity vary due to the presence of an inducible promoter which regulates GABA_{B}R1/R2 receptor expression.

This invention is directed to a method of determining the physiological effects of varying levels of activity of GABA_{B}R1/R2 receptors which comprises producing a panel of above-identified transgenic nonhuman mammals, each expressing a different amount of GABA_{B}R1/R2 receptor.

This invention is directed to a method for identifying an antagonist capable of alleviating an abnormality, by decreasing the activity of a GABA_{B}R1/R2 receptor comprising administering a compound to a above-identified transgenic nonhuman mammal, and determining whether the compound alleviates the physical and behavioral abnormalities displayed by the transgenic, nonhuman mammal, the alleviation of the abnormality identifying the compound as the antagonist.

This invention is directed to an antagonist identified by an above-identified method.

This invention is directed to a pharmaceutical composition comprising an above-identified antagonist and a pharmaceutically acceptable carrier.

This invention is directed to a method of treating an abnormality in a subject wherein the abnormality is alleviated by decreasing the activity of a GABA_{B}R1/R2 receptor which comprises administering to a subject an effective amount of an above-identified pharmaceutical composition, thereby treating the abnormality.

This invention is directed to a method for identifying an agonist capable of alleviating an abnormality, by increasing the activity of a GABA_{B}R1/R2 receptor comprising administering a compound to an above-identified transgenic nonhuman mammal, and determining whether the compound alleviates the physical and behavioral abnormalities displayed by the transgenic, nonhuman mammal, the alleviation of the abnormality identifying the compound as the agonist.

This invention is directed to an agonist identified by an above-identified method.

This invention is directed to a pharmaceutical composition comprising an above-identified agonist and a pharmaceutically acceptable carrier.

This invention is directed to a method for treating an abnormality in a subject wherein the abnormality is alleviated by increasing the activity of a GABA_{B}R1/R2 receptor which comprises administering to a subject an effective amount of an above-identified pharmaceutical composition, thereby treating the abnormality.

This invention is directed to a cell which expresses on its surface a mammalian GABA_{B}R1/R2 receptor that is not naturally expressed on the surface of such cell.

This invention is directed to a cell, wherein the mammalian GABA_{B}R1/R2 receptor comprises two polypeptides, one of which is a GABA_{B}R2 polypeptide and another of which is a GABA_{B}R1 polypeptide.

This invention is directed to a process for identifying a chemical compound which specifically binds to a GABA_{B}R1/R2 receptor which comprises contacting cells containing nucleic acid encoding and expressing on their cell surface the GABA_{B}R1/R2 receptor, wherein such cells do not normally express the GABA_{B}R1/R2 receptor, with the compound under conditions suitable for binding, and detecting specific binding of the chemical compound to the GABA_{B}R1/R2 receptor.

This invention is directed to a process for identifying a chemical compound which specifically binds to a GABA_{B}R1/R2 receptor which comprises contacting a membrane fraction from a cell extract of cells containing nucleic acid encoding and expressing on their cell surface the GABA_{B}R1/R2 receptor, wherein such cells do not normally express the GABA_{B}R1/R2 receptor, with the compound under conditions suitable for binding, and detecting specific binding of the chemical compound to the GABA_{B}R1/R2 receptor.

In one embodiment, the GABA_{B}R1/R2 receptor is a mammalian GABA_{B}R1/R2 receptor.

In another embodiment, the GABA_{B}R1/R2 receptor comprises a GABA_{B}R2 polypeptide which has substantially the same amino acid sequence as that encoded by the plasmid BO-55 (ATCC Accession No. 209104).

In another embodiment, the GABA_{B}R1/R2 receptor comprises a GABA_{B}R2 polypeptide which has substantially the same sequence as the amino acid sequence shown in FIGS. 23A-23D (SEQ ID NO:47).

In another embodiment, the GABA_{B}R1/R2 receptor comprises a GABA_{B}R2 polypeptide which has the amino acid sequence shown in FIGS. 23A-23D (SEQ ID NO:47).

In another embodiment, the GABA_{B}R1/R2 receptor comprises a GABA_{B}R2 polypeptide which has substantially the same amino acid sequence as that encoded by the plasmid pEXJT3T7-hGABAB2 (ATCC Accession No. 203515).

In another embodiment, the GABA_{B}R1/R2 receptor comprises a GABA_{B}R2 polypeptide which has substantially the same amino acid sequence as the sequence shown in FIGS. 23A-23D (SEQ ID NO:47).

In another embodiment, the GABA_{B}R1/R2 receptor comprises a GABA_{B}R2 polypeptide which has the sequence shown in FIGS. 23A-23D (SEQ ID NO:47).

In another embodiment, the compound is not previously known to bind to a GABA_{B}R1/R2 receptor.

This invention is directed to a compound identified by an above-identified process.

In one embodiment, the cell is an insect cell.

In another embodiment, the cell is a mammalian cell.

In another embodiment, the cell is nonneuronal in origin.

In another embodiment, the nonneuronal cell is a COS-7 cell, 293 human embryonic kidney cell, a CHO cell, a NIH-3T3 cell a mouse Y1 cell or LM(tk−) cell.

In another embodiment, the compound is not previously known to bind to a GABA_{B}R1/R2 receptor.

This invention is directed to a compound identified by an above-identified process.

This invention is directed to a process involving competitive binding for identifying a chemical compound which specifically binds to a GABA_{B}R1/R2 receptor which comprises separately contacting cells expressing on their cell surface the GABA_{B}R1/R2 receptor, wherein such cells do not normally express the GABA_{B}R1/R2 receptor, with both the chemical compound and a second chemical compound known to bind to the receptor, and with only the second chemical compound, under conditions suitable for binding of both compounds, and detecting specific binding of the chemical compound to the GABA_{B}R1/R2 receptor, a decrease in the binding of the second chemical compound to the GABA_{B}R1/R2 receptor in the presence of the chemical compound indicating that the chemical compound binds to the GABA_{B}R1/R2 receptor.

This invention is directed to a process involving competitive binding for identifying a chemical compound which specifically binds to a human GABA_{B}R1/R2 receptor which comprises separately contacting a membrane fraction from a cell extract of cells expressing on their cell surface the GABA_{B}R1/R2 receptor, wherein such cells do not normally express the GABA_{B}R1/R2 receptor, with both the chemical compound and a second chemical compound known to bind to the receptor, and with only the second chemical compound, under conditions suitable for binding of both compounds, and detecting specific binding of the chemical compound to the GABA_{B}R1/R2 receptor, a decrease in the binding of the second chemical compound to the GABA_{B}R1/R2 receptor in the presence of the chemical compound indicating that the chemical compound binds to the GABA_{B}R1/R2 receptor.

In one embodiment, the GABA_{B}R1/R2 receptor is a mammalian GABA_{B}R1/R2 receptor.

In another embodiment, the GABA_{B}R1/R2 receptor comprises a GABA_{B}R2 polypeptide which has substantially the same amino acid sequence as that encoded by plasmid BO-55 (ATCC Accession No. 209104).

In another embodiment, the GABA_{B}R1/R2 receptor comprises a GABA_{B}R2 polypeptide which has substantially the same amino acid sequence as that shown in FIGS. 23A-23D (SEQ ID NO:47).

In another embodiment, the GABA_{B}R1/R2 receptor comprises a GABA_{B}R2 polypeptide which has the amino acid sequence shown in FIGS. 23A-23D (SEQ ID NO:47).

In another embodiment, the GABA_{B}R1/R2 receptor comprises a GABA_{B}R2 polypeptide which has substantially the same amino acid sequence as that encoded by plasmid pEXJT3T7-hGABAB2 (ATCC Accession No. 203515).

In another embodiment, the GABA_{B}R1/R2 receptor comprises a GABA_{B}R2 polypeptide which has substantially the same amino acid sequence as the sequence shown in FIGS. 23A-23D (SEQ ID NO:47).

In another embodiment, the GABA_{B}R1/R2 receptor comprises a GABA_{B}R2 polypeptide which has the sequence shown in FIGS. 23A-23D (SEQ ID NO:47).

In another embodiment, the cell is an insect cell.

In another embodiment, the cell is a mammalian cell.

In another embodiment, the cell is nonneuronal in origin.

In another embodiment, the nonneuronal cell is a COS-7 cell, 293 human embryonic kidney cell, a CHO cell, a NIH-3T3 cell a mouse Y1 cell or LM(tk−) cell.

In another embodiment, the compound is not previously known to bind to a GABA_{B}R1/R2 receptor.

This invention is directed to a compound identified by an above-identified process.

This invention is directed to a method of screening a plurality of chemical compounds not known to bind to a GABA_{B}R1/R2 receptor to identify a compound which specifically binds to the GABA_{B}R1/R2 receptor, which comprises

(a) contacting cells containing nucleic acid encoding and expressing on their cell surface the GABA_{B}R1/R2 receptor, wherein such cells do not normally express the GABA_{B}R1/R2 receptor, with a compound known to bind specifically to the GABA_{B}R1/R2 receptor;

(b) contacting the same cells as in step (a) with the plurality of compounds not known to bind specifically to the GABA_{B}R1/R2 receptor, under conditions permitting binding of compounds known to bind the GABA_{B}R1/R2 receptor;

(c) determining whether the binding of the compound known to bind specifically to the GABA_{B}R1/R2 receptor is reduced in the presence of the plurality of the compounds, relative to the binding of the compound in the absence of the plurality of compounds, and if the binding is reduced;

(d) separately determining the extent of binding to the GABA_{B}R1/R2 receptor of each compound included in the plurality of compounds, so as to thereby identify the compound or compounds present in such plurality of compounds which specifically binds to the GABA_{B}R1/R2 receptor.

This invention is directed to a method of screening a plurality of chemical compounds not known to bind to a GABA_{B}R 1/R2 receptor to identify a compound which specifically binds to the GABA_{B}R1/R2 receptor, which comprises

(a) contacting a membrane fraction extract from cells containing nucleic acid encoding and expressing on their cell surface the GABA_{B}R1/R2 receptor, wherein such cells do not normally express the GABA_{B}R1/R2 receptor, with a compound known to bind specifically to the GABA_{B}R1/R2 is receptor;

(b) contacting the same membrane fraction as in step (a) with the plurality of compounds not known to bind specifically to the GABA_{B}R1/R2 receptor, under conditions permitting binding of compounds known to bind the GABA_{B}R1/R2 receptor;

(c) determining whether the binding of the compound known to bind specifically to the GABA_{B}R1/R2 receptor is reduced in the presence of the plurality of compounds, relative to the binding of the compound in the absence of the plurality of compounds, and if the binding is reduced;

(d) separately determining the extent of binding to the GABA_{B}R1/R2 receptor of each compound included in the plurality of compounds, so as to thereby identify the compound or compounds present in such plurality of compounds which specifically binds to the GABA_{B}R1/R2 receptor.

In one embodiment, the GABA_{B}R1/R2 receptor is a mammalian GABA_{B}R1/R2 receptor.

In one embodiment, the cell is a mammalian cell.

In one embodiment, the mammalian cell is non-neuronal in origin.

In one embodiment, the non-neuronal cell is a COS-7 cell, a 293 human embryonic kidney cell, a LM(tk−) cell, a CHO cell, a mouse Y1 cell or an NIH-3T3 cell.

This invention is directed to a process for determining whether a chemical compound is a GABA_{B}R1/R2 receptor agonist which comprises contacting cells with the compound under conditions permitting the activation of the GABA_{B}R1/R2 receptor, and detecting an increase in GABA_{B}R1/R2 receptor activity, so as to thereby determine whether the compound is a GABA_{B}R1/R2 receptor agonist.

This invention is directed to a process for determining whether a chemical compound is a GABA_{B}R1/R2 receptor antagonist which comprises contacting cells containing nucleic acid encoding and expressing on their cell surface the GABA_{B}R1/R2 receptor, wherein such cells do not normally express the GABA_{B}R1/R2 receptor, with the compound in the presence of a known GABA_{B}R1/R2 receptor agonist, under conditions permitting the activation of the GABA_{B}R1/R2 receptor, and detecting a decrease in GABA_{B}R1/R2 receptor activity, so as to thereby determine whether the compound is a GABA_{B}R1/R2 receptor antagonist.

Expression of genes in Xenopus oocytes is well known in the art (A. Cole*/man, Transcription and Translation: A Practical Approach (B. D. Hanes, S. J. Higgins, eds., pp 271-302, IRL Press, Oxford, 1984; Y. Masu et al., Nature 329:21583-21586, 1994) and is performed using microinjection of native mRNA or in vitro synthesized mRNA into frog oocytes. The preparation of in vitro synthesized mRNA can be performed by various standard techniques (J. Sambrook et al., Molecular Cloning: A Laboratory Manual, Second Edition, Cold Spring Harbor Laboratory, Cold Spring Harbor, N.Y., 1989) including using T7 polymerase with the mCAP RNA capping kit (Stratagene).

In one embodiment, the cells additionally express nucleic acid encoding GIRK1 and GIRK4.

In another embodiment, the GABA_{B}R2 receptor is a mammalian GABA_{B}R2 receptor.

This invention is directed to a pharmaceutical composition which comprises an amount of a GABA_{B}R1/R2 receptor agonist determined to be an agonist by an above-identified process effective to increase activity of a GABA_{B}R1/R2 receptor and a pharmaceutically acceptable carrier.

This invention is directed to a pharmaceutical, wherein the GABA_{B}R1/R2 receptor agonist was not previously known.

This invention is directed to a pharmaceutical composition which comprises an amount of a GABA_{B}R1/R2 receptor antagonist determined to be an antagonist an above-identified process effective to reduce activity of a GABA_{B}R1/R2 receptor and a pharmaceutically acceptable carrier.

This invention is directed to a pharmaceutical composition, wherein the GABA_{B}R1/R2 receptor antagonist was not previously known.

This invention is directed to a process for determining whether a chemical compound activates a GABA_{B}R1/R2 receptor, which comprises contacting cells producing a second messenger response and expressing on their cell surface the GABA_{B}R1/R2 receptor, wherein such cells do not normally express the GABA_{B}R1/R2 receptor, with the chemical compound under conditions suitable for activation of the GABA_{B}R1/R2 receptor, and measuring the second messenger response in the presence and in the absence of the chemical compound, a change in the second messenger response in the presence of the chemical compound indicating that the compound activates the GABA_{B}R1/R2 receptor.

In one embodiment, the second messenger response comprises potassium channel activation and the change in second messenger is an increase in the level of potassium current.

This invention is directed to a process for determining whether a chemical compound inhibits activation of a GABA_{B}R1/R2 receptor, which comprises separately contacting cells producing a second messenger response and expressing on their cell surface the GABA_{B}R1/R2 receptor, wherein such cells do not normally express the GABA_{B}R1/R2 receptor, with both the chemical compound and a second chemical compound known to activate the GABA_{B}R1/R2 receptor, and with only the second chemical compound, under conditions suitable for activation of the GABA_{B}R1/R2 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the GABA_{B}R1/R2 receptor.

In one embodiment, the second messenger response comprises potassium channel activation and the change in second messenger response is a smaller increase in the level of inward potassium current in the presence of both the chemical compound and the second chemical compound than in the presence of only the second chemical compound.

This invention is directed to an above-identified process, wherein the GABA_{B}R1/R2 receptor is a mammalian GABA_{B}R1/R2 receptor.

In one embodiment, the GABA_{B}R1/R2 receptor comprises a GABA_{B}R2 polypeptide which has substantially the same amino acid sequence as that encoded by the plasmid BO-55 (ATCC Accession No. 209104).

In another embodiment, the GABA_{B}R1/R2 receptor comprises a GABA_{B}R2 polypeptide which has substantially the same amino acid sequence as that shown in FIGS. 4A-4D (SEQ ID NO:4).

In another embodiment, the GABA_{B}R1/R2 receptor comprises a GABA_{B}R2 polypeptide which has substantially the same amino acid sequence as that shown in FIGS. 23A-23D (SEQ ID NO:47).

In another embodiment, the GABA_{B}R1/R2 receptor comprises a GABA_{B}R2 polypeptide which has the sequence, shown in FIGS. 23A-23D (SEQ ID NO:47).

In another embodiment, the GABA_{B}R1/R2 receptor comprises a GABA_{B}R2 polypeptide which has substantially the same amino acid sequence as that encoded by the plasmid pEXJT3T7-hGABAB2 (ATCC Accession No. 203515).

This invention is directed to an above-identified process, wherein the cell is an insect cell.

This invention is directed to an above-identified process, wherein the cell is a mammalian cell.

In one embodiment, the mammalian cell is nonneuronal in origin.

In another embodiment, the nonneuronal cell is a COS-7 cell, CHO cell, 293 human embryonic kidney cell, NIH-3T3 cell or LM(tk−) cell.

In another embodiment, the compound was not previously known to activate or inhibit a GABA_{B}R1/R2 receptor.

This invention is directed to a compound determined by an above-identified process.

This invention is directed to a pharmaceutical composition which comprises an amount of a GABA_{B}R1/R2 receptor agonist determined by an above-identified process effective to increase activity of a GABA_{B}R1/R2 receptor and a pharmaceutically acceptable carrier.

In one embodiment, the GABA_{B}R1/R2 receptor agonist was not previously known.

This invention is directed to a pharmaceutical composition which comprises an amount of a GABA_{B}R1/R2 receptor antagonist determined by an above-identified process effective to reduce activity of a GABA_{B}R1/R2 receptor and a pharmaceutically acceptable carrier.

In one embodiment, the GABA_{B}R1/R2 receptor antagonist was not previously known.

This invention is directed to method of screening a plurality of chemical compounds not known to activate a GABA_{B}R1/R2 receptor to identify a compound which activates the GABA_{B}R1/R2 receptor which comprises:

(a) contacting cells containing nucleic acid encoding and expressing on their cell surface the GABA_{B}R1/R2 receptor, wherein such cells do not normally express the GABA_{B}R1/R2 receptor, with the plurality of compounds not known to activate the GABA_{B}R1/R2 receptor, under conditions permitting activation of the GABA_{B}R1/R2 receptor;

(b) determining whether the activity of the GABA_{B}R1/R2 receptor is increased in the presence of the compounds, and if it is increased;

(c) separately determining whether the activation of the GABA_{B}R1/R2 receptor is increased by each compound included in the plurality of compounds, so as to thereby identify the compound or compounds present in such plurality of compounds which activates the GABA_{B}R1/R2 receptor.

In one embodiment, the cells express nucleic acid encoding GIRK1 and GIRK4.

In another embodiment, the GABA_{B}R1/R2 receptor is a mammalian GABA_{B}R1/R2 receptor.

This invention is directed to a method of screening a plurality of chemical compounds not known to inhibit the activation of a GABA_{B}R1/R2 receptor to identify a compound which inhibits the activation of the GABA_{B}R1/R2 receptor, which comprises:

(a) contacting cells containing nucleic acid encoding and expressing on their cell surface the GABA_{B}R1/R2 receptor, wherein such cells do not normally express the GABA_{B}R1/R2 receptor, with the plurality of compounds in the presence of a known GABA_{B}R1/R2 receptor agonist, under conditions permitting activation of the GABA_{B}R1/R2 receptor;

(b) determining whether the activation of the GABA_{B}R1/R2 receptor is reduced in the presence of the plurality of compounds, relative to the activation of the GABA_{B}R1/R2 receptor in the absence of the plurality of compounds, and if it is reduced;

(c) separately determining the inhibition of activation of the GABA_{B}R1/R2 receptor for each compound included in the plurality of compounds, so as to thereby identify the compound or compounds present in such a plurality of compounds which inhibits the activation of the GABA_{B}R1/R2 receptor.

In one embodiment, the cells express nucleic acid encoding GIRK1 and GIRK4.

In one embodiment, the GABA_{B}R1/R2 receptor is a mammalian GABA_{B}R1/R2 receptor.

In another embodiment, wherein the cell is a mammalian cell.

In another embodiment, the mammalian cell is non-neuronal in origin.

In another embodiment, the non-neuronal cell is a COS-7 cell, a 293 human embryonic kidney cell, a LM(tk−) cell or an NIH-3T3 cell.

This invention is directed to a pharmaceutical composition comprising a compound identified by an above-identified method, effective to increase GABA_{B}R1/R2 receptor activity and a pharmaceutically acceptable carrier.

This invention is directed to a pharmaceutical composition comprising a compound identified by an above-identified method, effective to decrease GABA_{B}R1/R2 receptor activity and a pharmaceutically acceptable carrier.

This invention is directed to a process for determining whether a chemical compound is a GABA_{B}R1/R2 receptor agonist, which comprises preparing a membrane fraction from cells which comprise nucleic acid encoding and expressing on their cell surface the GABA_{B}R1/R2 receptor, wherein such cells do not normally express the GABA_{B}R1/R2 receptor, separately contacting the membrane fraction with both the chemical compound and GTPΥS, and with only GTPΥS, under conditions permitting the activation of the GABA_{B}R1/R2 receptor, and detecting GTPΥS binding to the membrane fraction, an increase in GTPΥS binding in the presence of the compound indicating that the chemical compound activates the GABA_{B}R1/R2 receptor.

This invention is directed to a process for determining whether a chemical compound is a GABA_{B}R1/R2 receptor antagonist, which comprises preparing a membrane fraction from cells which comprise nucleic acid encoding and expressing on their cell surface the GABA_{B}R1/R2 receptor, wherein such cells do not normally express the GABA_{B}R1/R2 receptor, separately contacting the membrane fraction with the chemical compound, GTPΥS and a second chemical compound known to activate the GABA_{B}R1/R2 receptor, with GTPΥS and only the second compound, and with GTPΥS alone, under conditions permitting the activation of the GABA_{B}R1/R2 receptor, detecting GTPΥS binding to each membrane fraction, and comparing the increase in GTPΥS binding in the presence of the compound and the second compound relative to the binding of GTPΥS alone, to the increase in GTPΥS binding in the presence of the second chemical compound known to activate the GABA_{B}R1/R2 receptor relative to the binding of GTPΥS alone, a smaller increase in GTPΥS binding in the presence of the compound and the second compound indicating that the compound is a GABA_{B}R1/R2 receptor antagonist.

In one embodiment, the GABA_{B}R2 receptor is a mammalian GABA_{B}R2 receptor.

In another embodiment, the GABA_{B}R1/R2 receptor comprises a GABA_{B}R2 polypeptide which has substantially the same amino acid sequence as that encoded by the plasmid BO-55 (ATCC Accession No. 209104).

In another embodiment, the GABA_{B}R1/R2 receptor comprises a GABA_{B}R2 polypeptide which has substantially the same amino acid sequence as that shown in FIGS. 4A-4D (SEQ ID NO:4).

In another embodiment, the GABA_{B}R1/R2 receptor comprises a GABA_{B}R2 polypeptide which has substantially the same amino acid sequence as that encoded by the plasmid pEXJT3T7-hGABAB2 (ATCC Accession No. 203515).

In another embodiment, the GABA_{B}R1/R2 receptor comprises a GABA_{B}R2 polypeptide which has substantially the same amino acid sequence as that shown in FIGS. 23A-23D (SEQ ID NO:47).

In another embodiment, the GABA_{B}R1/R2 receptor comprises a GABA_{B}R2 polypeptide which has the sequence shown in FIGS. 23A-23D (SEQ ID NO:47).

In another embodiment, the cell is an insect cell.

In another embodiment, the cell is a mammalian cell.

In another embodiment, the mammalian cell is nonneuronal in origin.

In another embodiment, the nonneuronal cell is a COS-7 cell, CHO cell, 293 human embryonic kidney cell, NIH-3T3 cell or LM(tk−) cell.

In another embodiment, the compound was not previously known to be an agonist or antagonist of a GABA_{B}R1/R2 receptor.

This invention is directed to a compound determined to be an agonist or antagonist of a GABA_{B}R1/R2 receptor by an above-identified process.

This invention is directed to a method of treating spasticity in a subject which comprises administering to the subject an amount of a compound which is an agonist of a GABA_{B}R1/R2 receptor effective to treat spasticity in the subject.

This invention is directed to a method of treating asthma in a subject which comprises administering to the subject an amount of a compound which is a GABA_{B}R1/R2 receptor agonist effective to treat asthma in the subject.

This invention is directed to a method of treating incontinence in a subject which comprises administering to the subject an amount of a compound which is a GABA_{B}R1/R2 receptor agonist effective to treat incontinence in the subject.

This invention is directed to method of decreasing nociception in a subject which comprises administering to the subject an amount of a compound which is a GABA_{B}R1/R2 receptor agonist effective to decrease nociception in the subject.

This invention is directed to a use of a GABA_{B}R2 agonist as an antitussive agent which comprises administering to the subject an amount of a compound which is a GABA_{B}R1/R2 receptor agonist effective as an antitussive agent in the subject.

This invention is directed to a method of treating drug addiction in a subject which comprises administering to the subject an amount of a compound which is a GABA_{B}R1/R2 receptor agonist effective to treat drug addiction in the subject.

This invention directed to a method of treating Alzheimer's disease in a subject which comprises administering to the subject an amount of a compound which is a GABA_{B}R1/R2 receptor antagonist effective to treat Alzheimer's disease in the subject.

This invention is directed to a peptide selected from the group consisting of:

| | |
|---|---|
| a) P L Y S I L S A L T I L G M I M A S A F L F F N I | (SEQ ID NO:48) |
| K N; | |
| b) L I I L G G M L S Y A S I F L F G L D G S F V S E | (SEQ ID NO:49) |
| K T; | |
| c) C T V R T W I L T V G Y T T A F G A M F A K T W | (SEQ ID NO:50) |
| R; | |
| d) Q K L L V I V G G M L L I D L C I L I C W Q; | (SEQ ID NO:51) |
| e) M T I W L G I V Y A Y K G L L M L F G C F L A | (SEQ ID NO:52) |
| W; | |
| f) A L N D S K Y I G M S V Y N V G I M C I I G A A V; | (SEQ ID NO:53) and |
| g) C I V A L V I I F C S T I T L C L V F V P K L I T | (SEQ ID NO:54) |
| L R T N. | |

This invention is directed to a compound that prevents the formation of a GABA_{B}R1/R2 receptor complex.

Transmembrane peptides derived from GABA_{B}R2 sequences may modulate the functional activity of GABA_{B}R1/R2 receptors. One mode of action involves the destruction of the GABA_{B}R1/R2 receptor complex via competitive displacement of the GABA_{B}R2 polypeptide subunit by the peptide upon binding to the GABA_{B}R1 polypeptide subunit. The peptides may be synthesized using standard solid phase F-moc peptide synthesis protocol using an Advanced Chemtech 396 Automated Peptide Synthesizer.

Additional GABA_{B} subtypes in hypothalamus and caudate putamen are predicted due to the under-representation of GABA_{B}R2 hybridization signals. These novel GABA_{B} proteins and others may be identified by using GABA_{B}R2 polypeptides in co-immunoprecipitation experiments.

This invention provides a process for making a composition of matter which specifically binds to a GABA_{B}R1/R2 receptor which comprises identifying a chemical compound using any of the processes described herein for identifying a compound which binds to and/or activates or inhibits activation of a GABA_{B}R1/R2 receptor and then synthesizing the chemical compound or a novel structural and functional analog or homolog thereof. In one embodiment, the GABA_{B}R1/R2 receptor is a human GABA_{B}R1/R2 receptor.

This invention further provides a process for preparing a pharmaceutical composition which comprises admixing a pharmaceutically acceptable carrier and a pharmaceutically acceptable amount of a chemical compound identified by any of the processes described herein for identifying a compound which binds to and/or activates or inhibits activation of a GABA_{B}R1/R2 receptor or a novel structural and functional analog or homolog thereof. In one embodiment, the GABA_{B}R1/R2 receptor is a human GABA_{B}R1/R2 receptor.

Thus, once the gene for a targeted receptor subtype is cloned, it is placed into a recipient cell which then expresses the targeted receptor subtype on its surface. This cell, which expresses a single population of the targeted human receptor subtype, is then propagated resulting in the establishment of a cell line. This cell line, which constitutes a drug discovery system, is used in two different types of assays: binding assays and functional assays. In binding assays, the affinity of a compound for both the receptor subtype that is the target of a particular drug discovery program and other receptor subtypes that could be associated with side effects are measured. These measurements enable one to predict the potency of a compound, as well as the degree of selectivity that the compound has for the targeted receptor subtype over other receptor subtypes. The data obtained from binding assays also enable chemists to design compounds toward or away from one or more of the relevant subtypes, as appropriate, for optimal therapeutic efficacy. In functional assays, the nature of the response of the receptor subtype to the compound is determined. Data from the functional assays show whether the compound is acting to inhibit or enhance the activity of the receptor subtype, thus enabling pharmacologists to evaluate compounds rapidly at their ultimate human receptor subtypes targets permitting chemists to rationally design drugs that will be more effective and have fewer or substantially less severe side effects than existing drugs.

Approaches to designing and synthesizing receptor subtype-selective compounds are well known and include traditional medicinal chemistry and the newer technology of combinatorial chemistry, both of which are supported by computer-assisted molecular modeling. With such approaches, chemists and pharmacologists use their knowledge of the structures of the targeted receptor subtype and compounds determined to bind and/or activate or inhibit activation of the receptor subtype to design and synthesize structures that will have activity at these receptor subtypes.

Combinatorial chemistry involves automated synthesis of a variety of novel compounds by assembling them using different combinations of chemical building blocks. The use of combinatorial chemistry greatly accelerates the process of generating compounds. The resulting arrays of compounds are called libraries and are used to screen for compounds (lead compounds) that demonstrate a sufficient level of activity at receptors of interest. Using combinatorial chemistry it is possible to synthesize focused libraries of compounds anticipated to be highly biased toward the receptor target of interest.

Once lead compounds are identified, whether through the use of combinatorial chemistry or traditional medicinal chemistry or otherwise, a variety of homologs and analogs are prepared to facilitate an understanding of the relationship between chemical structure and biological or functional activity. These studies define structure activity relationships which are then used to design drugs with improved potency, selectivity and pharmacokinetic properties. Combinatorial chemistry is also used to rapidly generate a variety of structures for lead optimization. Traditional medicinal chemistry, which involves the synthesis of compounds one at a time, is also used for further refinement and to generate compounds not accessible by autometed techniques. Once such drugs are defined the production is scaled up using standard chemical manufacturing methodiologies utilized throughout the pharmaceutical and chemistry industry.

This invention will be better understood from the Experimental Details which follow. However, one skilled in the art will readily appreciate that the specific methods and results discussed are merely illustrative of the invention as described more fully in the claims which follow thereafter.

### Experimental Details

### Materials and Methods

### DNA Sequencing

DNA sequences were determined using an ABI PRISM 377 DNA Sequencer (Perkin-Elmer, Foster City, Calif.) according to the manufacturer's instructions.

### Hybridization Methodology

Probes were end-labeled with polynucleotide kinase according to the manufacturer's instructions (Boehringer-Mannheim). Hybridization was performed on Zeta-Probe membrane (Bio-Rad, CA) at reduced stringency: 40° C. in a solution containing 25% formamide, 5×SSC (1×SSC=0.15 M NaCl, 0.015 M sodium citrate), 1×Denhardt's solution (0.02% polyvinylpyrrolindone, 0.02% Ficoll, 0.02% bovine serum albumin) and 25 μg/μL sonicated salmon sperm DNA. Membrane strips were washed at 40° C. in 0.1×SSC containing 0.1% SDS and exposed at −70° C. to Kodak XAR film in the presence of an intensifying screen.

The nucleotide sequences of the hybridization probes are shown below:

| | |
|---|---|
| T-891: 5′-AGGGATGCTTTCCTATGCTTCCATATTTCTCTTTGGCCTTGATGG-3′ | (SEQ ID NO:5) |
| Nucleotides 1449-1493 of TL-267, forward strand. | |
| T-892: 5′-CAATGTGCAGTTCTGCATCGTGGCTCTGGTCATCATCTTCTGCAG-3′ | (SEQ ID NO:6) |
| Nucleotides 2022-2066 of TL-267, forward strand. | |

### PCR Methodology

PCR reactions were carried out using a PE 9600 (Perkin-Elmer) PCR cycler in 20 μL volumes using Expand Long Template Polymerase (Boehringer-Mannheim) and the manufacturer's buffer 1 for internal PCR primers or manufacturer's buffer 2 for vector-anchored PCR. Reactions were run using a program consisting of 35 cycles of 94° C. for 30 sec., 68° C. for 20 sec, and 72° C. for 1 min, with a pre-incubation at 95° C. for 5 min and post-incubation hold at 4° C.

Nucleotide sequences of the primer sets used in PCR reactions are shown below:

| T-94: | 5′-CTTCTAGGCCTGTACGGAAGTGTT-3′ | (SEQ ID NO: |
|---|---|---|
| vector, forward primer. | | 7); |
| T-95: | 5′-GTTGTGGTTTGTCCAAACTCATCAAT-3′ | (SEQ ID NO: |
| vector, reverse primer. | | 8); |
| T-887: | 5′-GGGATGAGTGTCTACAACGTGGGG-3′ | (SEQ ID NO: |
| nucleotides 1948-1971 of TL-267, forward primer. | | 9); |
| T-888: | 5′-TGCGTTGCTGCATCTGGGTTTGTTCT-3′ | (SEQ ID NO: |
| nucleotides 2138-2113 of TL-267, reverse primer. | | 10); |
| T-889: | 5′-ATCTCCCTACCTCTCTACAGCATCCT-3′ | (SEQ ID NO: |
| nucleotides 1300-1325 of TL-267, forward primer. | | 11); |
| T-890: | 5′-CAGGTCCTGACGGTGCAAAGTGTTTC-3′ | (SEQ ID NO: |
| nucleotides 1544-1519 of TL-267, reverse primer. | | 12); |
| T-921: | 5′-TGACGCAAGACGTTCAGAGGTTCTCT-3′ | (SEQ ID NO: |
| nucleotides 473-498 of TL-267, forward primer. | | 13); |
| T-922: | 5′-TGTAGCCTTCCATGGCAGCAAGCAGA-3′ | (SEQ ID NO: |
| nucleotides 814-789 of TL-267, reverse primer. | | 14); |
| T-923: | 5′-AGAGAACCTCTGAACGTCTTGCGTCA-3′ | (SEQ ID NO: |
| nucleotides 498-473 of TL-267, reverse primer. | | 15); |
| T-935: | 5′-GGCTCTGTTGTGTTCCACTGTAGCTG-3′ | (SEQ ID NO: |
| nucleotides 2483-2458 of TL-267, reverse primer. | | 16); |
| +TLR,1 T-938: | 5′-TCATGCCGCTCACCAAGGAGGTGGCC-3′ | (SEQ ID NO: |
| nucleotides 53 to 78 of TL-267, forward primer. | | 17); |
| T-939: | 5′-GGCCACCTCCTTGGTGAGCGGCATGA-3′ | (SEQ ID NO: |
| nucleotides 78 to 53 of TL-267, reverse primer. | | 18); |
| T-947: | 5′-TGAGTGAGCAGAGTCCAGAGCCGT-3′ | (SEQ ID NO: |
| nucleotides −68 to −45 of TL-267, forward primer. | | 19); |
| T-948: | 5′-ATGGATGGGAGGTAGGCGTGGTGGAG-3′ | (SEQ ID NO: |

| | |
|---|---|
| nucleotides 2591-2566 of TL-267, reverse primer. | 20); |

### Preparation of Human Hippocampal CDNA Library

Total RNA was prepared by a modification of the guanidine thiocyanate method, from 6 grams of human hippocampus. Poly A⁺RNA was purified with a FastTrack kit (Invitrogen Corp., San Diego, Calif.). Double stranded (ds) cDNA was synthesized from 4 μg of poly A⁺ RNA according to Gübler and Hoffman (1983), except that ligase was omitted in the second strand cDNA synthesis. The resulting DS cDNA was ligated to BstxI/EcoRI adaptors (Invitrogen Corp.), the excess of adaptors was removed by exclusion chromatography. High molecular weight fractions were ligated in pcEXV.BS (An Okayama and Berg expression vector) cut by BstxI as described by Aruffo and Seed (1987). The ligated DNA was electroporated in E. coli MC 1061 (Gene Pulser, Biorad). A total of 2.2×10⁶ independent clones with an insert mean size of approximately 3 kb was generated. The library was plated on Petri dishes (Ampicillin selection) in pools of 0.4 to 1.2×10⁴ independent clones. After 18 hours amplification, the bacteria from each pool were scraped, resuspended in 4 mL of LB media and 1.5 mL processed for plasmid purification by the alkali method (Sambrook et al, 1989). 1 mL aliquots of each bacterial pool were stored at −85° C. in 20% glycerol.

### BLAST Search that Identified a Novel 7-TM Protein Sequence

Sequence analysis was performed with the Wisconsin Package Version 9.0, Genetics Computer Group (GCG), Madison, Wis. The rat GABA_{B}R1a amino acid sequence (Kaupmann et al. (1997) Nature 386:239) was used as a query to search the EST division of GenBank with BLAST. Two entries, T07621 and Z43654, had probability scores that suggested significant amino acid homology to the GABA_{B}R1a polypeptide. T07621 had sequence homology from the beginning of the first transmembrane domain to the beginning of third transmembrane domain of the GABA_{B}R1a polypeptide. Z43654 had sequence homology from the sixth transmembrane domain to the seventh transmembrane domain of the GABA_{B}R1a polypeptide. The sequence documentation for T07621 and Z43654 was retrieved with Entrez (NCBI) and neither sequence was annotated as having homology to any 7-TM spanning protein.

### T07621 and Z43654 are Part of the Same Sequence

A series of PCR reactions were carried out on human hippocampus DNA with multiple primer sets: primer set T-887/T-888 designed to Z43654 sequence; primer set T-889/T-890 designed to the T07621 sequence; and primer set T-889/T-888 designed to the forward strand of T07621 and the reverse stand of Z43654. The PCR products was loaded on duplicate lanes of an agarose gel and the DNA was southern blotted to a Zeta-Probe membrane (Bio-Rad, CA). The regions of the membrane corresponding to the individual lanes on the gel were cut to produce membrane strips that contained duplicate samples of the DNA. One set of membrane strips was hybridized with T-891, a probe specific for the T07621 sequence. Another set of membranes was hybridized with T-892, a probe specific to the Z43654 sequence. The membrane from primer set T-887/T-888 hybridized with probe T-892 for the Z43654 sequence. The membrane from primer set T-889/T-890 hybridized with probe T-891 for the T07621 sequence. The membrane from primer set T889/T-888 hybridized with both the T-891 and T-892 probes.

### Isolating the Full-length Human cDNA by PCR Sib Selection

PCR reactions were carried out on bacterial pools containing a human hippocampus cDNA library. Primer set T-888/T-889 was used to identify the bacterial pools that contained a portion of the novel receptor. Vector-anchored PCR was carried out on the positive pools to determine which pool contained the longest cDNA insert. Four primer sets were used for the vector-anchored PCR: T-94/T-888, T-94/T889, T-95/T888, and T-95/T889. Pool 365 was identified having the longest cDNA inset and the plasmid was sib selected (McCormick, 1987). The nucleotide sequence of clone 365-9-7-4, designated TL-260, was translated into amino acids and compared to the amino acid sequence of the rat GABA_{B}R1a polypeptide. Relative the rat GABA_{B}R 1a amino acid sequence, TL-260 was truncated at the amino terminus.

A set of PCR primers (T-921/T-922) was made to the 5′ region of TL-260 and was used to re-screen the bacterial pools of the human hippocampus library for the missing segment of the novel clone. Vector-anchored PCR was carried out on the positive pools to determine which pool contained the longest cDNA insert. Four primer sets were used for the vector-anchored PCR: T-94/T-921, T-94/T922, T-95/T921, and T-95/T-922. Pool 299 contained the most 5′ sequence. A PCR product derived from the primer set T-94/T-923 was isolated (T-261) and sequenced. The putative amino acids derived from TL-261 were compared to the rat GABA_{B}R1 sequence. TL-261 contained an initiation codon but didn't contain a stop codon upstream of the initiation codon.

A set of PCR primers (T-938/T-935) was made to the 5′region of TL-261 and was used to re-screen the bacterial pools of the human hippocampus library for additional sequence. Vector-anchored PCR was carried out on the positive pools to determine which pool contained the longest cDNA insert. Four primer sets were used for the vector-anchored PCR: T-94/T-938, T-94/T939, T-95/T938, and T-95/T-939. A PCR product derived from primer set T-95/T-939 was isolated (T-261a) and sequenced. The putative amino acids derived from T-261a were compared to the rat GABA-1 amino acid sequence. T-261a contained an initiation codon and an in-frame upstream stop codon.

From the vector-anchored PCR, pool 389 contained the longest cDNA insert. This pool was sib selected with the primer set T-947/T-935. The resulting plasmid, 389-20-29-2, was designated TL-266 and was sequenced.

### Construction of GABA_{B}R2 Polypeptide in Expression Vector

A Cla-I-Xba-I fragment from TL-266 was subcloned into the expression vector pEXJ.HRT3T7 and designated TL-267. This plasmid (TL-267) was deposited on Jun. 10, 1997, with the American Type Culture Collection (ATCC), 12301 Parklawn Drive, Rockville, Md. 20852, U.S.A. under the provisions of the Budapest Treaty for the International Recognition of the Deposit of Microorganisms for the Purposes of Patent Procedure and was accorded ATCC Accession No. 209103.

### Generation of Rat GABA_{B}R2 PCR Product

cDNA from rat hippocampus and rat cerebellum were amplified in 50 μL PCR reaction mixtures using the Expand Long Template PCR System (as supplied and described by the manufacturer, Boehringer Mannheim) using a program consisting of 40 cycles of 94° C. for 1 min, 50° C. for 2 min, and 68° C. for 2 min, with a pre- and post-incubation of 95° C. for 5 min and 68° C. for 7 min, respectively. PCR primers for rat GABA_{B}R2 were designed against the human GABA_{B}R2 sequence: BB 257, forward primer in the first transmembrane domain, and BB 258, reverse primer in the seventh transmembrane domain. The single 780 bp fragment from both rat hippocampus and rat cerebellum were isolated from a 1% agarose gel, purified using a GENECLEAN III kit (BIO 101, Vista, Calif.) and sequenced using AmpliTaq DNA Polymerase, FS (Perkin Elmer). The sequence was run on an ABI PRISM 377 DNA Sequencer and analyzed using the Wisconsin Package (GCG, Genetics Computer Group, Madison, Wis.). This sequence was used to design PCR primers for the rat GABA_{B}R2 gene.

### Construction and Screening of a Rat Hypothalamic cDNA Library

Poly A⁺ RNA was purified from rat hypothalamic RNA (Clontech) using a FastTrack kit (Invitrogen, Corp.). DS-cDNA was synthesized from 5 μg of poly A+ RNA according to Gubler and Hoffman (1983) with minor modifications. The resulting cDNA was ligated to BstXI adaptors (Invitrogen, Corp.) And the excess adapters removed by exclusion column chromatography. High molecular weight fractions of size-selected ds-cDNA were ligated in pEXJ.T7, an Okayama and Berg expression vector modified from pcEXV (Miller and Germain, 1986) to contain BstXI, other additional restriction sites, and a T7 promoter. A total of 100,000 independent clones with a mean insert size of 3.7 kb were generated. The library was amplified on agar plates (Ampicillin selection) in 48 primary pools. Glycerol stocks of the primary pools screened for a rat GABA_{B}R2 gene by PCR using BB265, a forward primer from the loop between transmembrane domains 3 and 4 from the sequence determined above and BB266, a reverse primer from the sixth transmembrane domain from the sequence determined above. The conditions for PCR were 1 min at 94° C., 4 min at 68° C. for 40 cycles, with a pre- and post-incubation of 5 min at 95° C. and 7 min at 68° C., respectively. To determine which pools had the largest inserts, positive pools were screened by PCR using the vector primers BB172 or BB173, and a gene-specific primer BB265 or BB266. One positive primary pool, I-47, was subdivided into 24 pools of 1000 clones, and grown in LB medium overnight. Two μL of cultures were screened by PCR using primers BB172 and BB266. One positive subpool, I-47-4 was subdivided into 10 pools of 200 clones and plated on agar plates (ampicillin selection). Colonies were transferred to nitrocellulose membranes (Schleicher and Schuell, Keene, N.H.), denatured in 0.4 N NaOH, 1.5 M NaCl, renatured in 1M Tris, 1.5 M NaCl, and UV cross-linked. Filters were hybridized overnight at 40° C. in a buffer containing 50 % formamide, 0.12 M Na₂HPO₄ (pH7.2), 0.25M NaCl, 7%SDS, 25 mg/L ssDNA and 10⁶ cpm/mL of a cDNA probe corresponding to transmembrane domains 1 to 7 of rat GABA_{B}R2, labeled with [³²P]dCTP (3000 Ci/mmol, NEN) using a random prime labeling kit (Boehringer Mannheim). Filters were washed 1×5 min then 2×20 min at room temperature in 2×SSC, 0.1% SDS then 3×20 min at 500 in 0.1×SSC, 0.1% SDS and exposed to Biomax MS film (Kodak) for 3 hours. Four closely clustering colonies which appeared to hybridize were re-screened individually by PCR using primers BB265 and BB266, primers BB265 and BB55, primers BB265 and BB56, and primers BB266 and BB55. The conditions for PCR were 30 sec at 94° C., 2.5 min at 68° C. for 32 cycles, with a pre- and post-incubation of 5 min at 95° C. and 5 min at 68° C. respectively. One positive colony, I-47-4-2, was amplified overnight in 10 mL TB media and processed for plasmid purification using a standard alkaline lysis miniprep procedure followed by a PEG precipitation. This plasmid was designated BO54 and partially sequenced using AmpliTaq DNA Polymerase, FS (Perkin Elmer). The sequence was run on an ABI PRISM 377 DNA Sequencer and analyzed using the Wisconsin Package (GCG, Genetics Computer Group, Madison, Wis.). BO54 was in the wrong orientation for expression in mammalian cells. To obtain a clone in the correct orientation, an EcoRI restriction fragment from BO54 was subcloned into the vector pEXJ. Transformants were screened by PCR using the primers BB56 and BB268 under the following conditions: 30 sec at 94° C., 2.5 min at 68° C. for 32 cycles, with a pre- and post-incubation of 5 min at 95° C. and 3 min at 68° C. respectively. One transformant in the correct orientation was amplified overnight in 100 ml

TB media and processed for plasmid purification using a standard alkaline lysis miniprep procedure followed by a PEG precipitation. This plasmid was designated BO55 and sequenced using AmpliTaq DNA Polymerase, FS (Perkin Elmer). Plasmid BO-55 was deposited with the ATCC on Jun. 10, 1997, and was accorded ATCC Accession No. 209104. The sequence of BO-55 was determined using an ABI PRISM 377 DNA Sequencer and analyzed using the Wisconsin Package (GCG, Genetics Computer Group, Madison, Wis.).

### Primers Used

| | | |
|---|---|---|
| BB257: | 5′-CTCTCTGCCCTCACCATCCTCGGGAT-3′ | (SEQ ID NO:21) |
| BB258: | 5′-GACTCCGGCTCGAATACCAGGCAGAG-3′ | (SEQ ID NO:22) |
| BB265: | 5′-CCATGTTTGCAAAGACCTGGAGGGTCC-3′ | (SEQ ID NO:23) |
| BB266: | 5′-GGTCACGCGTCAGGAAAGAGACAGCAG-3′ | (SEQ ID NO:24) |
| BB172: | 5′-AAGCTTCTAGAGATCCCTCGACCTC-3′ | (SEQ ID NO:25) |
| BB173: | 5′-AGGCGCAGAACTGGTAGGTATGGAA-3′ | (SEQ ID NO:26) |
| BB55: | 5′-CTTCTAGGCCTGTACGGAAGTGTTA-3′ | (SEQ ID NO:27) |
| BB56: | 5′-GTTGTGGTTTGTCCAAACTCATCAATG-3′ | (SEQ ID NO:28) |
| BB268: | 5′-CTGCTGTCTCTTTCCTGACGCGTGACC-3′ | (SEQ ID NO:29). |

### Generation of DNA Coding for Rat GABA_{B}1b and GABA_{B}1a Polylpeptides

The gene encoding the rat GABA_{B}R1b polypeptide was obtained by screening the same rat hypothalamic library used for GABA_{B}R2 with primers based on the original publication of the clone by Kaupmann, et al., 1997. A partial clone lacking the first 55 nucleotides was identified and ligated to a PCR fragment containing the missing base pairs to obtain the full length clone. A restriction fragment containing the entire coding region of GABA_{B}R1b was subcloned into the mammalian expression vector pEXJ.T7 and designated “BO58”. A rat GABA_{B}1a polypeptide clone was obtained by ligating a restriction fragment of the GABA_{B}1b clone, which contained the common region of the GABA_{B}1 gene, to a PCR product containing the GABA_{B}1a-specific 5′ end.

### In Situ Hybridization Experiments for GABA_{B}R2 mRNA Animals

Male Sprague-Dawley rats (Charles Rivers, Rochester, N.Y.) were euthanized using C0₂, decapitated, and their brains immediately removed and rapidly frozen on crushed dry ice. Coronal sections of brain tissue were cut at 11 μm using a cryostat and thaw-mounted onto poly-L-lysine-coated slides and stored at −20° C. until use.

### Tissue Preparation

Prior to hybridization, the tissues were fixed in 4% paraformaldehyde/PBS pH 7.4 followed by two washes in PBS (Specialty Media, Lavallette, N.J.). Tissues were then treated in 5 mM dithiothreitol, rinsed in DEPC-treated PBS, acetylated in 0.1 M triethanolamine containing 0.25% acetic anhydride, rinsed twice in 2×SSC, delipidated with chloroform then dehydrated through a series of graded alcohols. All reagents were purchased from Sigma (St. Louis, Mo.).

### Radioactive In Situ Hybridization Histochemistry

Oligonucleotide probes, MJ79/80, corresponding to nucleotides 354-398 and MJ109/110, corresponding to nucleotides 952-991 of the rat GABA_{B}R2 cDNA, MJ94/95, corresponding to nucleotides 151-193 of the human GABA_{B}R1a cDNA, and MJ83/84, corresponding to nucleotides 34-71 of the rat GABA_{B}R1b cDNA were used to characterize the distribution of each polypeptides's respective mRNA. The oligonucleotides were synthesized using an Expedite Nucleic Acid Synthesis System (PerSeptive Biosystems, Framingham, Mass.) and purified using 12% polyacrylamide gel electrophoresis. Additionally, sense and antisense oligonucleotides corresponding to positions 1076-1120 of GABA_{B}R1b (1424-1468 of GABA_{B}R1a) were used (BB403 and BB404).

The sequences of the oligonucleotides are:

For rat GABA_{B}R2:

| | | |
|---|---|---|
| Sense probe, | MJ79: | |
| | 5′ - GCA ATA AAG TAT GGG CTG AAC CAT TTG | (SEQ ID NO:36) |
| | ATG GTG TTT GGA GGC GT - 3′ | |
| Antisense probe, | MJ8O: | |
| | 5′ - ACG CCT CCA AAC ACC ATC AAA TGG TTC | (SEQ ID NO:37) |
| | AGC CCA TAC TTT ATT GC - 3′ | |
| Sense probe, | MJ1O9: | |
| | 5′ - TTT GAG CCC CTG AGC TCC AAA CAA ATC | (SEQ ID NO:38) |
| | AAG ACC ATC TCA G - 3′ | |
| Antisense probe, | MJ11O: | |
| | 5′ - CTG AGA TGG TCT TGA TTT GTT TGG AGC | (SEQ ID NO:39) |
| | TCA GGG GCT CAA A - 3′ | |
| For human GABA8R1a: | | |
| Sense probe, | MJ94: | |
| | 5′ - AAG GCC ATC AAC TTC CTG CCT GTG GAC | (SEQ ID NO:40) |
| | TAT GAG ATC GAA TAT G - 3′ | |
| Antisense probe, | MJ95: | |
| | 5′ - CAT ATT CGA TCT CAT AGT CCA CAG GCA | (SEQ ID NO:41) |
| | GGA AGT TGA TGG CCT T - 3′ | |
| For rat GABA8R1b: | | |
| Sense probe, | MJ83: | |
| | 5′ - TGG CCG CTG CCT CTT CTG CTG GTG ATG | (SEQ ID NO:42) |
| | GCG GCT GGG GT - 3′ | |
| Antisense probe, | MJ84: | |
| | 5′ - ACC CCA GCC GCC ATC ACC AGC AGA AGA | (SEQ ID NO:43) |
| | GGC AGC GGC CA - 3′ | |
| Sense probe, | BB403: | |
| | 5′ - CCT TGG CTT TGG CCT TGA ACA AGA CGT | (SEQ ID NO:44) |
| | CTG GAG GAG GTG GTC GTT - 3′ | |
| Antisense probe, | BB404: | |
| | 5′ - AAC GAC CAC CTC CTC CAG ACG TCT TGT | (SEQ ID NO:45) |
| | TCA AGG CCA AAG CCA AGG - 3′ | |

Probes were 3′-end labeled with [³⁵S]dATP (1200Ci/mmol, NEN, Boston, Mass.) to a specific activity of 109 dpm/pg using terminal deoxynucleotidyl transferase (Pharmacia, Piscataway, N.J.). In situ hybridization was done with modification of the method described by Durkin, M, et al, 1995.

### Nonradioactive In Situ Hybridization Histochemistry

Antisense/sense probes corresponding to nucleotides 354-398 of the rat GABA_{B}R2 cDNA, were 3′-end labeled with digoxigenin using TdT. The labeling reaction was carried out as outlined in the DIG/Genius System, (Boehringer Mannheim, Indianapolis, Ind.). Conditions used in ISHH with digoxigenin-labeled probes are the same as described above. The sections were rinsed in buffer 1, washing buffer (0.1 M Tris-HCl pH 7.5/0.15 M NaCl), pre-incubated in Blocking Solution (Buffer 1, 0.1% Triton-X and 2% normal sheep serum) for 30 minutes and then incubated for 2 hours in Blocking Solution containing anti-digoxigenin-AP Fab fragment (Boehringer Mannheim) at 1:500 dilution followed by two 10 minute washes in Buffer 1. To develop color, sections were rinsed in Detection Buffer (0.1M Tris-HCl pH 9.5/0.15M NaCl/0.05 M MgCl₂) for 10 minutes and then incubated overnight in Detection Buffer containing 0.5 mM NBT, 0.1 mM BCIP, and 1 mM levamisole. After color development, slides were dipped in dH2O and coverslipped using aqua mount.

Probe specificity was established by performing in situ hybridization on HEK293 cells transiently transfected with eukaryotic expression vectors containing the rat GABA_{B}R1b and human GABA_{B}R1a DNA or no insert for transfection. Furthermore, two pairs of hybridization probes, sense and antisense, that were targeted to different segments of the GABA_{B}R2 mRNA were used for cells and rat tissues.

### Quantification

The strength of the hybridization signal obtained in various region of the rat brain was graded as weak (+), moderate (++), heavy (+++) or intense (++++). These were qualitative evaluations for each of the polypeptide mRNA distributions based on the relative optical density on the autoradiographic film and on the relative number of silver grains observed over individual cells at the microscopic level.

### Cell Culture

COS-7 cells are grown on 150 mm plates in DMEM with supplements (Dulbecco's Modified Eagle Medium with 10% bovine calf serum, 4 mM glutamine, 100 units/mL penicillin/100 Ag/mL streptomycin) at 37° C., 5% CO₂. Stock plates of COS-7 cells are trypsinized and split 1:6 every 3-4 days.

Human embryonic kidney 293 cells are grown on 150 mm plates in DMEM with supplements (10% bovine calf serum, 4 mM glutamine, 100 units/mL penicillin/100 μg/mL streptomycin) at 37° C., 5% CO₂. Stock plates of 293 cells are trypsinized and split 1:6 every 3-4 days.

Mouse fibroblast LM(tk−) cells are grown on 150 mm plates in D-MEM with supplements (Dulbecco's Modified Eagle Medium with 10% bovine calf serum, 4 mM glutamine, 100 units/mL penicillin/100 μg/mL streptomycin) at 37° C., 5% CO₂. Stock plates of LM(tk−) cells are trypsinized and split 1:10 every 3-4 days.

Chinese hamster ovary (CHO) cells are grown on 150 mm plates in HAM's F-12 medium with supplements (10% bovine calf serum, 4 mM L-glutamine and 100 units/mL penicillin/100 ug/mL streptomycin) at 37° C., 5% CO2. Stock plates of CHO cells are trypsinized and split 1:8 every 3-4 days.

Mouse embryonic fibroblast NIH-3T3 cells are grown on 150 mm plates in Dulbecco's Modified Eagle Medium (DMEM) with supplements (10% bovine calf serum, 4 mM glutamine, 100 units/mL penicillin/100 μg/mL streptomycin) at 37° C., 5% CO2. Stock plates of NIH-3T3 cells are trypsinized and split 1:15 every 3-4 days.

Sf9 and Sf21 cells are grown in monolayers on 150 mm tissue culture dishes in TMN-FH media supplemented with 10% fetal calf serum, at 27° C., no CO₂. High Five insect cells are grown on 150 mm tissue culture dishes in Ex-Cell 400™ medium supplemented with L-Glutamine, also at 27° C., no CO₂.

LM(tk−) cells stably transfected with the DNA encoding the polypeptides disclosed herein may be routinely converted from an adherent monolayer to a viable suspension. Adherent cells are harvested with trypsin at the point of confluence, resuspended in a minimal volume of complete DMEM for a cell count, and further diluted to a concentration of 10⁶ cells/mL in suspension media (10% bovine calf serum, 10% 10× Medium 199 (Gibco), 9 mM NaHCO₃, 25 mM glucose, 2 mM L-glutamine, 100 units/mL penicillin/100 μg/mL streptomycin, and 0.05% methyl cellulose). Cell suspensions are maintained in a shaking incubator at 37° C., 5% CO₂ for 24 hours. Membranes harvested from cells grown in this manner may be stored as large, uniform batches in liquid nitrogen.

Alternatively, cells may be returned to adherent cell culture in complete DMEM by distribution into 96-well microtiter plates coated with poly-D-lysine (0.01 mg/mL) followed by incubation at 37° C., 5% CO₂ for 24 hours.

### Generation of Baculovirus

The coding region of DNA encoding the polypeptides disclosed herein may be subcloned into pBlueBacIII into existing restriction sites, or sites engineered into sequences 5′ and 3′ to the coding region of the polypeptides. To generate baculovirus, 0.5 μg of viral DNA (BaculoGold) and 3 μg of DNA construct encoding a polypeptide may be co-transfected into 2×10⁶ Spodoptera frugiperda insect Sf9 cells by the calcium phosphate co-precipitation method, as outlined in by Pharmingen (in “Baculovirus Expression Vector System: Procedures and Methods Manual”) The cells then are incubated for 5 days at 27° C.

The supernatant of the co-transfection plate may be collected by centrifugation and the recombinant virus plaque purified. The procedure to infect cells with virus, to prepare stocks of virus and to titer the virus stocks are as described in Pharmingen's manual.

### Transfection

All subtypes studied may be transiently transfected into COS-7 cells by the DEAE-dextran method, using 1 μg of DNA/10⁶ cells (Cullen, 1987). In addition, Schneider 2 Drosophila cells may be cotransfected with vectors containing the gene, under control of a promoter which is active in insect cells, and a selectable resistance gene, eg., the G418 resistant neomycin gene, for expression of the polypeptides disclosed herein.

### Stable Transfection

DNA encoding the polypeptides disclosed herein may be co-transfected with a G-418 resistant gene into the human embryonic kidney 293 cell line by a calcium phosphate transfection method (Cullen, 1987). Stably transfected cells are selected with G-418.

### Radioligand Binding Assays

Transfected cells from culture flasks were scraped into 5 mL of Tris-HCl, 5 mM EDTA, pH 7.5, and lysed by sonication. The cell lysates were centrifuged at 1000 rpm for 5 min. at 4° C., and the supernatant was centrifuged at 30,000×g for 20 min. at 4° C. The pellet was suspended in binding buffer (50 mM Tris-HCl, 2.5 mM CaCl₂ at pH 7.5 supplemented with 0.1% BSA, 2 μg/mL aprotinin, 0.5 mg/mL leupeptin, and 10 μg/mL phosphoramidon). Optimal membrane suspension dilutions, defined as the protein concentration required to bind less than 10% of the added labeled compound (typically a radiolabeled compound), were added to 96-well polypropylene microtiter plates containing labeled compound, unlabeled compounds (i.e., displacing ligand in an equilibrium competition binding assay) and binding buffer to a final volume of 250 μL. In equilibrium saturation binding assays membrane preparations were incubated in the presence of increasing concentrations of labeled compound. The binding affinities of the different compounds were determined in equilibrium competition binding assays, using labeled compound, such as 1 nM [³H]-CGP54626, in the presence of ten to twelve different concentrations of the displacing ligand(s). Some examples of displacing ligands included GABA, baclofen, 3APMPA, phaclofen, CGP54626, and CGP55845. Mixtures of several unlabeled test compounds (up to about 10 compounds) may also be used in competition binding assays, to determine whether one of the mixture component compounds binds to the polypeptide or receptor. Binding reaction mixtures were incubated for 1 hr at 30° C., and the reaction was stopped by filtration through GF/B filters treated with 0.5% polyethyleneimine, using a cell harvester. Where the labeled compound was a radiolabeled compound, the amount of bound compound was evaluated by gamma counting (for ¹²⁵I) or scintillation counting (for ³H). Data were analyzed by a computerized non-linear regression program. Non-specific binding was defined as the amount of radioactivity remaining after incubation of membrane protein in the presence of excess unlabeled compound. Protein concentration may be measured by the Bradford method using Bio-Rad Reagent, with bovine serum albumin as a standard.

### Cyclic AMP (cAMP) Formation Assay

The receptor-mediated inhibition of cyclic AMP (cAMP) formation may be assayed in transfected cells expressing the mammalian receptors described herein. Cells are plated in 96-well plates and incubated in Dulbecco's phosphate buffered saline (PBS) supplemented with 10 mM HEPES, 5 mM theophylline, 2 μg/ml aprotinin, 0.5 mg/ml leupeptin, and 10 μg/ml phosphoramidon for 20 min at 37° C., in 5% CO₂. Test compounds are added and incubated for an additional 10 min at 37° C. The medium is then aspirated and the reaction stopped by the addition of 100 mM HCl. The plates are stored at 4° C. for 15 min, and the cAMP content in the stopping solution measured by radioimmunoassay. Radioactivity may be quantified using a gamma counter equipped with data reduction software.

### Generation of Chimeric G-proteins

Chimeric G-proteins were constructed using standard mutagenesis methods (Conklin et al., 1993). Two chimeras were constructed. The first comprises the entire coding region of human Gα_{q} with the exception of the final 3′ 15 nucleotides which encode the C-terminal 5 amino acids of Gαᵢ₃. The second also comprises the entire coding region of human Gα_{q} with the exception of the final 3′ 15 nucleotides which encode the C-terminal 5 amino acids of Gα_{z}. Sequences of both chimeric G-protein genes were verified by nucleotide sequencing. For the purposes of expression in oocytes, synthetic mRNA transcripts of each gene were synthesized using the T7 polymerase.

### Phosphoinositide Assay

The agonist activities of GABA-B agonists were assayed by measuring their ability to generate phosphoinositide production in COS-7 cells transfected transiently with GABA_{B}R1, GABA_{B}R2, and chimeric Gα_{q/z}. Alternatively, COS-7 cells are transfected transiently with GABA_{B}R 1, GABA_{B}R2, and other chimeric G-protein alpha subunits such as Gα_{q/i2}, Gα_{q/i3}, or Gα_{q/o}. Cells were plated in 96-well plates and grown to confluence. The day before the assay the growth medium was changed to 100 μl of medium containing 1% serum and 0.5 μCi [³H]myo-inositol, and the plates were incubated overnight in a CO₂ incubator (5% CO₂ at 37° C.).

Immediately before the assay, the medium was removed and replaced by 200 μl of PBS containing 10 mM LiCl, and the cells were equilibrated with the new medium for 20 min. The [³H]inositol-phosphate (IP) accumulation was started by adding 22 μl of a solution containing the agonist. To the first two wells 22 μl of PBS were added to measure basal accumulation, and 10 different concentrations of agonist were assayed in the following 10 wells of each plate row. All assays were performed in duplicate by repeating the same additions in two consecutive rows. The plates were incubated in a CO₂ incubator for 30 min. The reaction was terminated by removal of the buffer solution by blotting, followed by the addition of 100 μl of 50% (v/v) trichloroacetic acid (TCA), and 10 min incubation at 4° C.

The contents of the wells were then transferred to a Multiscreen HV filter plate (Millipore) containing Dowex AG1-X8 (200-400 mesh, formate form). The filter plates were prepared adding 100 μl of Dowex AG1-X8 suspension (50% v/v, water:resin) to each well. The filter plates were placed on a vacuum manifold to wash or elute the resin bed. Each well was washed 3 times with 200 μl of 5 mM myo-inositol. The [³H]-IPs were eluted into empty 96-well plates with 75 μl of 1.2 M ammonium formate/0.1 M formic acid. After the addition of 200 μl of scintillation cocktail (Optiphase Supermix; Wallac) to each well, [³H]-Ips were quantified by counting on a Trilux 1450 Microbeta scintillation counter.

### Oocyte Expression

Female Xenopus laevis (Xenopus-1, Ann Arbor, Mich.) are anesthetized in 0.2% tricain (3-aminobenzoic acid ethyl ester, Sigma Chemical Corp.) and a portion of ovary is removed using aseptic technique (Quick and Lester, 1994). Oocytes are defolliculated using 3 mg/ml collagenase (Worthington Biochemical Corp., Freehold, N.J.) in a solution containing 87.5 mM NaCl, 2 mM KCl, 2 mM MgCl₂ and 5 mM HEPES, pH 7.5. Oocytes are injected (Nanoject, Drummond Scientific, Broomall, Pa.) with 50-70 nl mRNA prepared as described below. After injection of mRNA, oocytes are incubated at 17 degrees for 3-8 days.

RNAs are prepared by transcription from: (1), linearized DNA plasmids containing the complete coding region of the gene, or (2), templates generated by PCR incorporating a T7 promoter and a poly A⁺ tail. From either source, DNA is transcribed into mRNA using the T7 polymerase (“Message Machine”, Ambion).

The transcription template for the rat GABA_{B}R1b gene was prepared by PCR amplification of the plasmid BO58 using the primers MJ23 and MJ47 (see below). The template for the rat GABA_{B}R2 gene was made by linearization of the plasmid BO56, rat GABA_{B}R2 insert from BO55 in the expression vector pEXJ.T7, with NotI.

Primers:

| | |
|---|---|
| MJ23 5′ CCAAGCTTCTAATACGACTCACTATAGGGGAGACCATGGGCCCGGGGGG | (SEQ ID NO:30); |
| ACCCTGTACC 3′ | |
| MJ47 5′ T₍₃₅₎CACTTGTAAAGCAAATGTACTCGACTCC 3′ | (SEQ ID NO:31). |

Genes encoding G-protein inwardly rectifying K⁺ channels 1 and 4 (GIRK1 and GIRK4; “GIRKs”) were obtained by PCR using the published sequences (Kubo et al., 1993; Dascal et al., 1993; Krapivinsky et al., 1995b) to derive appropriate 5′ and 3′ primers. Human heart cDNA was used as template together with the primers

| | |
|---|---|
| 5′ -CGCGGATCCATTATGTCTGCACTCCGAAGGAAATTTG-3′ | (SEQ ID NO:32) and |
| 5′ -CGCGAATTCTTATGTGAAGCGATCAGAGTTCATTTTTC-3′ | (SEQ ID NO:33) for GIRK1 and |
| 5′ -GCGGGATCCGCTATGGCTGGTGATTCTAGGAATG-3′ | (SEQ ID NO:34) and |
| 5′ -CCGGAATTCCCCTCACACCGAGCCCCTGG-3′ | (SEQ ID NO:35) for GIRK4. |

The BamH1 and EcoR1 restriction sites in each primer pair were used to clone the PCR product into the expression vector pcDNA-Amp (Invitrogen). Plasmid vectors containing GIRK1 and GIRK4 are referred to as “JS1800” and “JS1741”, respectively. The coding regions of both genes were sequenced and verified.

### Oocyte Electrophysiology

Dual electrode voltage clamp (“GeneClamp”, Axon Instruments Inc., Foster City, Calif.) is performed using 3 M KCl-filled glass microelectrodes having resistances of 1-3 Mohms. Unless otherwise specified, oocytes are voltage clamped at a holding potential of −80 mV. During recordings, oocytes are bathed in continuously flowing (1-3 ml/min) medium containing 96 mM NaCl, 2 mM KCl, 1.8 mM CaCl₂, 1 mM MgCl₂, and 5 mM HEPES, pH 7.5 (ND96), or elevated K⁺ containing 49 mM KCl, 49 mM NaCl, 1.8 mM CaCl₂, 2 mM MgCl₂, and 5 mM HEPES, pH 7.5 (hK). Drugs are applied either by local perfusion from a 10 μl glass capillary tube fixed at a distance of 0.5 mm from the oocyte, or for calculation of steady-state EC₅₀s, by switching from a series of gravity fed perfusion lines. Experiments are carried out at room temperature. All values are expressed as mean +/− standard error of the mean.

Concentration-response curves for agonists and antagonists were fitted with logistic equations of the form I=1/(1+(EC₅₀/[Agonist])ⁿ) for agonists and I=1/(1+([Antagonist]/IC₅₀)ⁿ) for antagonists, where I is current, where EC₅₀ is the concentration of agonist that produced half-maximal activation, IC₅₀ is the concentration of antagonist that produced half-maximal inhibition, and n the Hill coefficient. Fits were made with a Marquardt-Levenberg non-linear least-squares curve fitting algorithm.

### Recording Ion Currents in Mammalian Cells

The ability of the rat GABA_{B}R1 and GABA_{B}R2 genes to activate GIRK currents in mammalian cells was investigated by transient transfection of HEK-293 cells followed by voltage clamp analysis of currents. HEK-293 cells were maintained in Dulbecco's modified Eagle medium (DMEM) plus 10% (v/v) bovine calf serum, 2% L-glutamine, 50 U/ml penicillin, and 50 μg/ml streptomycin and were incubated at 37° C. in a humidified 5% CO₂ atmosphere. Cells were harvested twice each week by treatment with 0.25% trypsin/1 mM EDTA in Hank's Salts and re-seeded at 20% of their original density either into 75 cm² flasks (for passaging) or into 35 mm tissue culture dishes (for transfection and electrophysiology experiments).

HEK-293 cells, 40%-80% confluent, were co-transfected with various combinations of 0.6 ug each of the following plasmids: pGreen Lantern-1 (Gibco/BRL, Gaithersburg, Md.), human GIRK1 (JS1800), human GIRK4 (JS1741), rat GABA_{B}R1b (BO58), and rat GABA_{B}R2 (BO55). Cells were transiently transfected using the Superfect Transfection Reagent from Qiagen (Valencia, Calif.) according to the manufacturer's instructions. Briefly, 3 μg total plasmid DNA were incubated with 22.5 μl Superfect Reagent in 100 μl serum-free DMEM for 5-10 minutes at room temperature. After addition of 600 μl complete DMEM, the DNA/Superfect mixture was transferred to cells growing in 35 mm dishes coated with poly-D-lysine and incubated for 2-4 hours at 37° C. in a 5% CO₂ incubator. Subsequently, the dishes were washed once with phosphate-buffered saline and 2 ml complete DMEM was added. Cells were incubated for 24-72 hours at 37° C. before performing electrophysiological measurements.

The whole-cell configuration of the patch-clamp technique was used with glass pipettes having resistances of 2-4 MΩ when filled with the pipette solution. Solutions used were (in mM), KMeSO₄, 125; KCl, 5; NaCl, 5; MgCl₂, 2; EGTA, 11; HEPES, 10, pH 7.4; MgATP, 1.0; Na₂GTP, 0.2, for the pipette and NaCl, 130; KCl, 4; CaCl₂, 2; MgCl₂, 2; Glucose, 10; Sucrose, 10; HEPES, 10, pH 7.4 for the bath. GIRK currents were recorded in elevated K⁺ solution containing 25 mM K⁺ and a correspondingly lower concentration of Na⁺. Voltage clamp recordings were made with an EPC-9 amplifier using Pulse+PulseFit software (HEKA Elektronik). Series resistances were kept below 10 Mohm and no attempt was made to provide series resistance compensation. Currents were low-pass filtered at 1 kHz and digitized at a rate of 5 kHz. Unless otherwise noted, experiments were performed at room temperature on cells voltage clamped at a holding potential of −70 mV. Application of agonists was realized using a gravity-fed, perfusion system consisting of six concentrically arranged microcapillary tubes (Jones et al. 1997). The time to complete solution exchange was about 100 ms. The bath was constantly perfused at a low rate with control solution.

All voltage clamp recordings were made from transfected cells visualized under epifluorescent lighting conditions utilizing a filter set designed for GFP (Zeiss Optics) Fluorescent cells were an excellent indication of transfection since they all exhibited some constitutive GIRK current activity in contrast to untransfected cells which displayed no measurable inward rectifier K⁺ currents (data not shown).

### Microphysiometry

GABA_{B}R1, GABA_{B}R2 or the combination, were transiently expressed in CHO-K1 cells by liposome mediated transfection according to the manufacturer's recommendations (“LipofectAMINE”, GibcoBRL, Bethesda, Md.), and maintained in Ham's F-12 medium with 10% bovine serum. Cells were prepared for microphysiometric recording as previously described (Salon, J. A., et al., 1995). On the day of the experiment the cell capsules were transferred to the microphysiometer and allowed to equilibrate in recording media (low buffer RPMI 1640, no bicarbonate, no serum, Molecular Devices Corp.), during which a baseline was established. The recording paradigm consisted of a 100 ml/min flow rate and a 30 s flow interruption during which the rate measurement was taken. Challenges involved an 80 s drug exposure just prior to the first post-challenge rate measurement being taken, followed by two additional pump cycles. Acidification rates reported are expressed as a percentage increase of the peak response over the baseline rate observed just prior to challenge.

### N-terminal Deletion Experiments

As a start to exploring the structural aspects of GABA_{B}R2 important for functional activity of the GABA_{B}R1/R2 receptor, N-terminal deletion experiments were performed on the GABA_{B}R2-HA construct (see below). All such deletion mutants caused a complete disruption of receptor activity as assessed by the measurement of GIRK currents in transfected HEK293 cells. In one such experiment, wildtype GABA_{B}R2-HA was digested with BglII restriction enzyme and religated. The BglII deletion mutant (M118) lacks 257 amino acids at the N-terminus, corresponding to positions 226-482. Using immunofluorescence, M118 was found to be expressed on the cell surface, similarly to the wildtype GABA_{B}R2-HA, yet when co-expressed with GABA_{B}R1 did not produce GIRK activation with 100 μM GABA. Thus, although we cannot yet identify specific amino acids contributing to receptor activity, it appears that the N-terminal region comprising amino acids 226-482 is critically important either for dimer formation, ligand binding or conformational changes associated with signal transduction.

### Construction of Epitope-tagged Polypeptides and Confocal Microscopy

Incorporation of sequences encoding the RGS6xHis or influenza virus hemagglutinin (HA) epitope into the GABA_{B}R1 and GABA_{B}R2 genes, respectively, was performed by PCR. Each epitope was positioned immediately before the stop codon in the appropriate gene. Both tagged genes were subcloned into pcDNA. Sequence analysis was used to confirm all PCR-derived portions of the construct. Forty-eight hours post-transfection HEK293 cells were fixed for 20 min in 4% paraformaldehyde in PBS, permeablized in PBS containing 2% BSA and 0.1% Triton X-100 and incubated with primary antibody for 1.5 h. Mouse monoclonal anti-RGS (Qiagen) and mouse anti-FLAG (Boehringer-Mannheim) were labeled with FITC-conjugated goat anti-mouse antibodies. Rat monoclonal anti-HA (Boehringer-Mannheim) was visualized with TRITC-conjugated rabbit anti-irat antibodies. Fluorescent images were obtained with a Zeiss LSM 410 confocal microscope using a 100× oil-immersion objective.

### Immunoprecipitation and Western Blotting

Forty-eight hours following transient transfection HEK293 cells were solubilized in lysis buffer containing (in mM): 50 Tris/Cl pH 7.4, 300 NaCl, 1.5 MgCl₂, 1 CaCl₂, protease inhibitors (Boehringer Mannheim tablets), 1% Triton X-100, and 10% glycerol. 1-2 mg of protein was immunoprecipitated overnight at 4° C. with either 0.5 μg rat monoclonal anti-HA antibody or 0.5 μg mouse monoclonal anti-4xHis antibody (Qiagen). Immune complexes were bound to 20 μl Protein-A agarose (Research Diagnostics, Inc.) for 2 h at RT. Protein-A pellets were washed twice with buffer containing Triton-X-100, then once without, and eluted with 80 μl Laemmli sample buffer containing 2% (w/v) SDS and 20 mM DTT. After heating for 3 min. at 70° C., 20 μl IP samples or 20 μg total protein was subjected to SDS-PAGE followed by Western blotting with either anti-HA or anti-4xHis antibody, followed by sheep anti-rat (Amersham) or goat anti-mouse (RDI) HRP-linked secondary antibodies, respectively. Proteins were visualized with enhanced chemiluminescent substrates (Pierce).

Alternatively, material for immunoprecipitations was obtained by sucrose gradient fractionation of the P1 pellet as described by Graham(Graham, 1984). To verify the enrichment of plasma membrane in the resulting “P1+” pellet, Na⁺/K⁺ ATPase in the P1+ and P2 (primarily microsomal and vesicular(Graham, 1984)) fractions was quantified by fluorescence detection of anti-alpha 1 subunit antibody (Research Diagnostics, Inc., clone 9A-5) on a phosphor imager (Molecular Dynamics). ATPase in P1+ fractions used for immunoprecipitations was found to be enriched >50 fold compared to P2 fractions.

### Experimental Results

### Novel GPCR Sequences Identified by BLAST Search

The rat GABA_{B}R1a amino acid sequence (Kaupmann et al. (1997) Nature 386:239) was used as a query to search the EST division of GenBank with BLAST. Two entries, T07621 and Z43654, had probability scores that suggested significant amino acid homology to the GABA_{B}R1a polypeptide. T07621 had sequence homology from the beginning of the first transmembrane domain to the beginning of third transmembrane domain of the GABA_{B}R1a polypeptide. Z43654 had sequence homology from the sixth transmembrane domain to the seventh transmembrane domain of the GABA_{B}R1a polypeptide. The sequence documentation for T07621 and Z43654 was retrieved with Entrez (NCBI) and neither sequence was annotated as having homology to any 7-TM spanning protein.

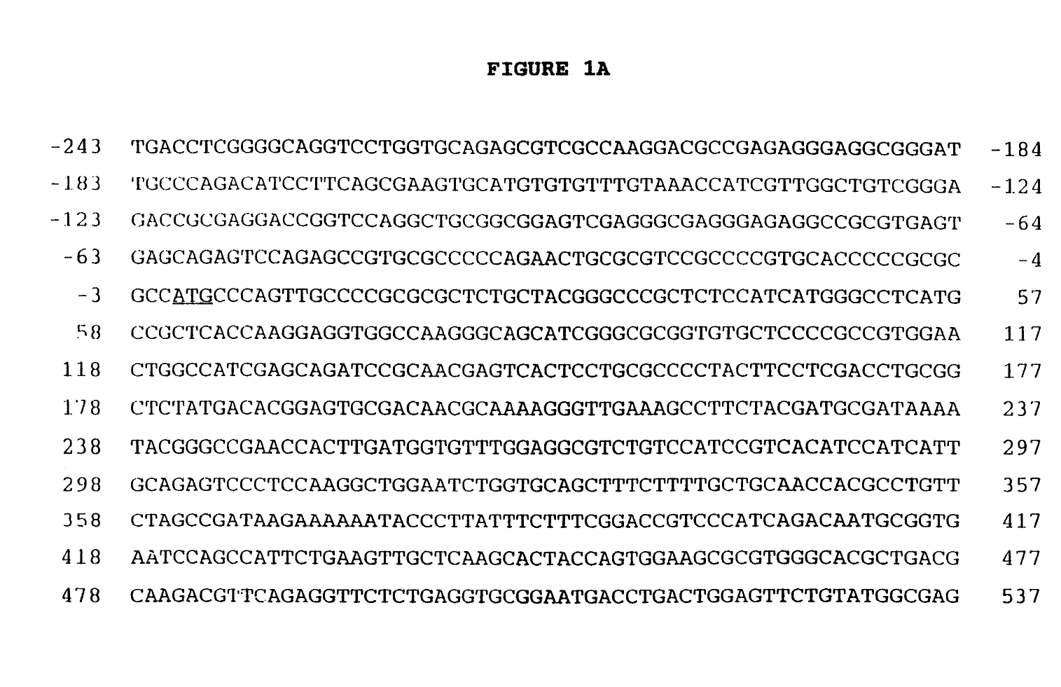

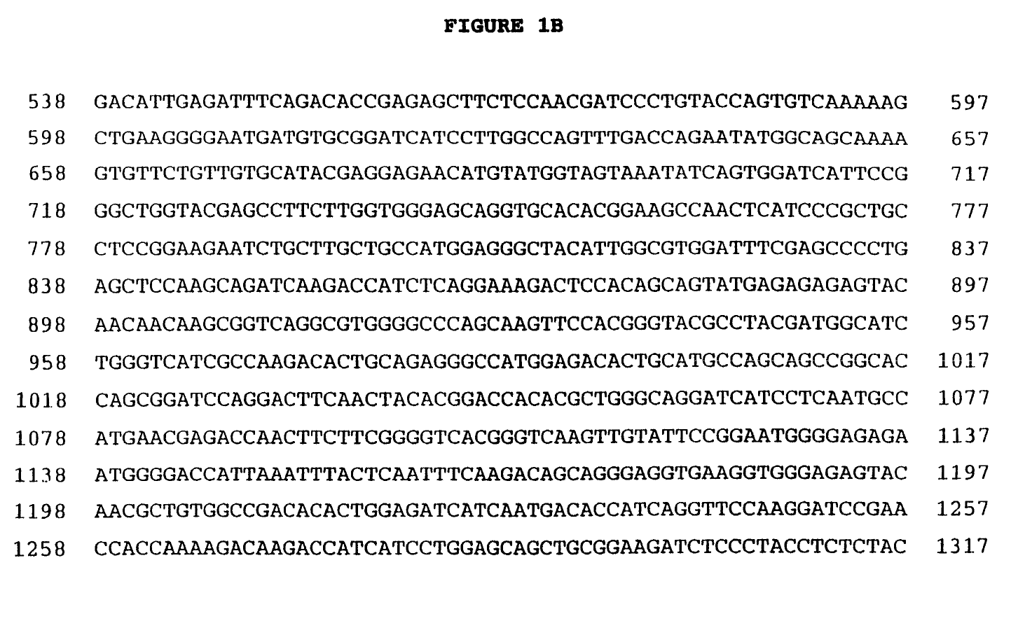

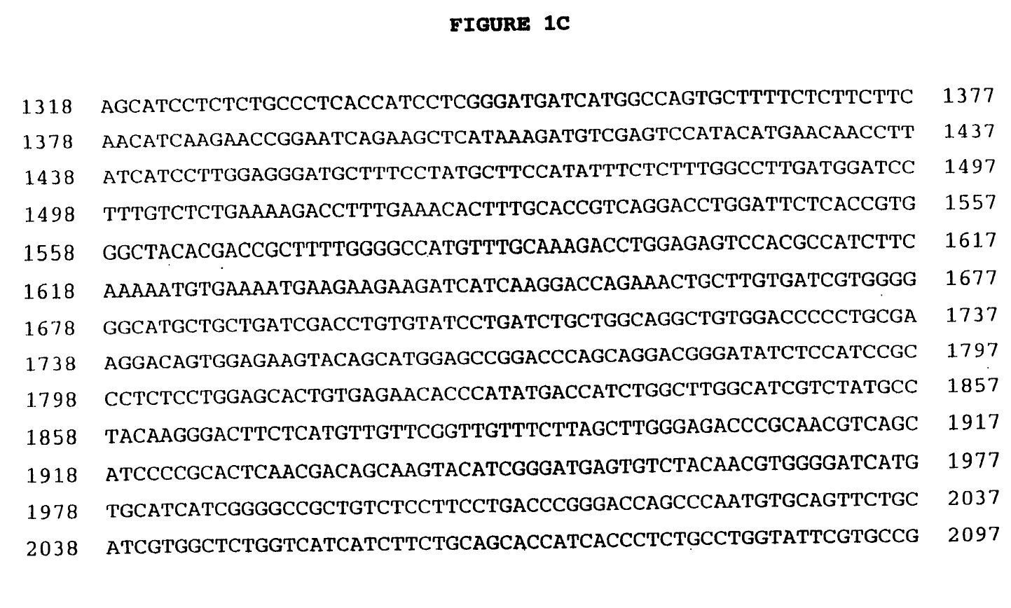

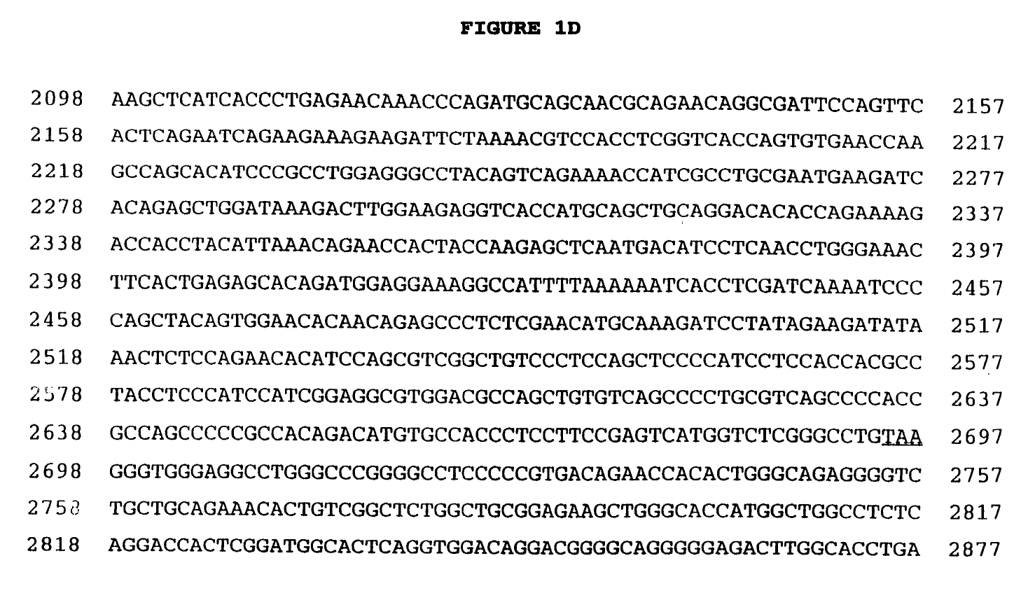

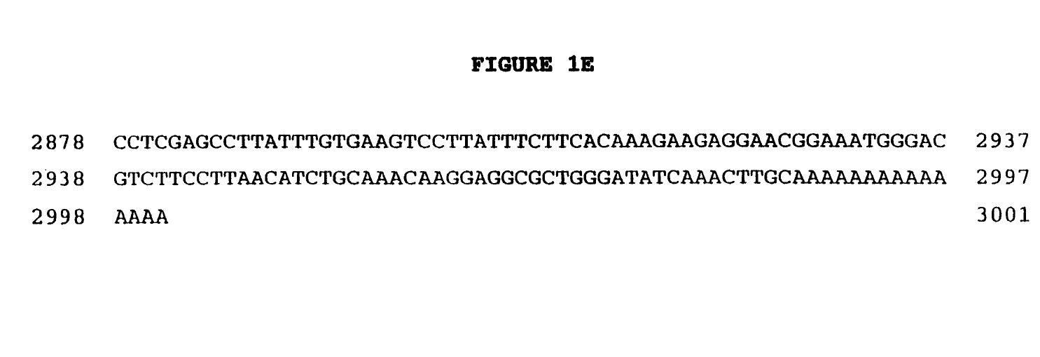

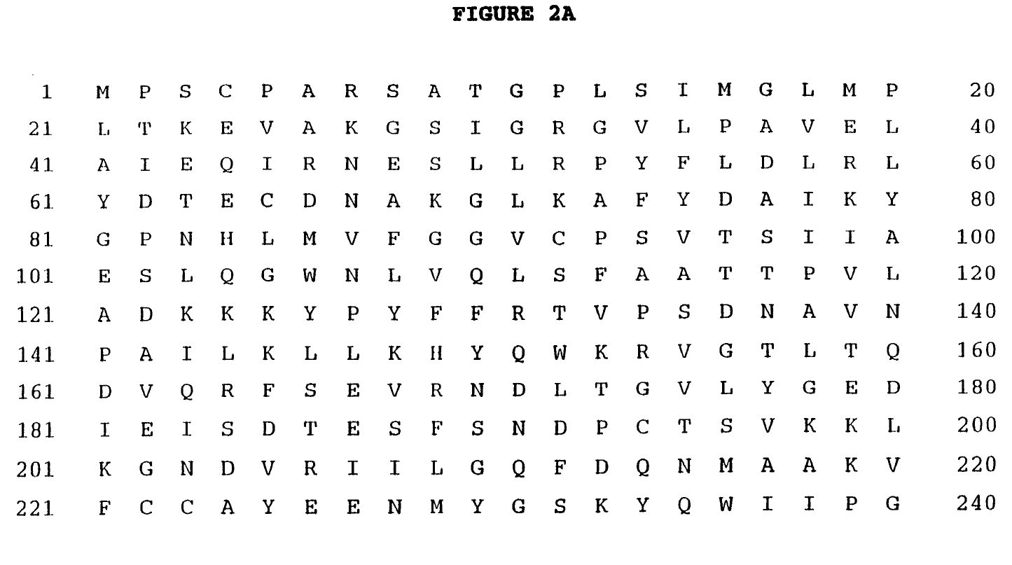

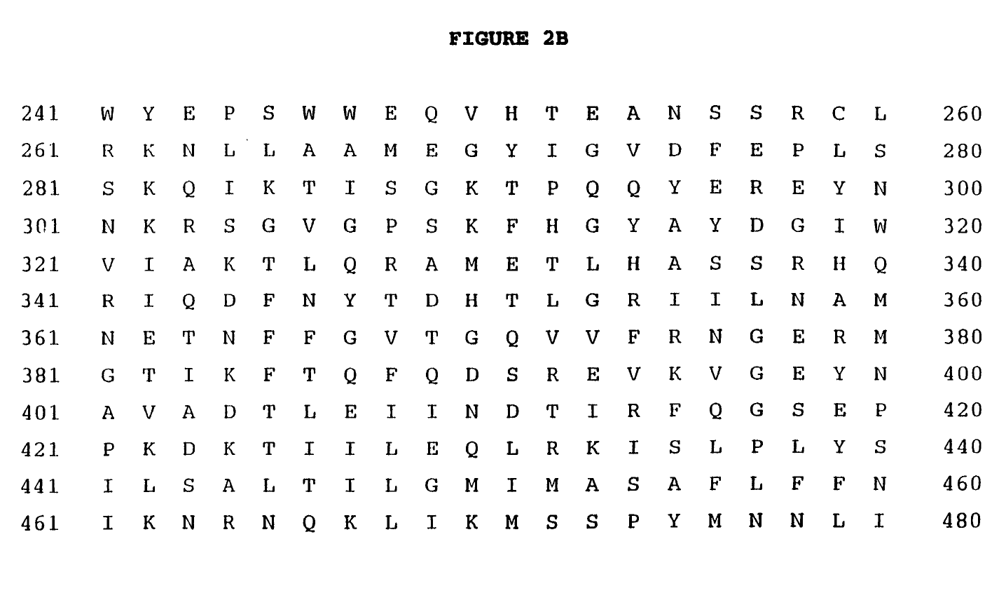

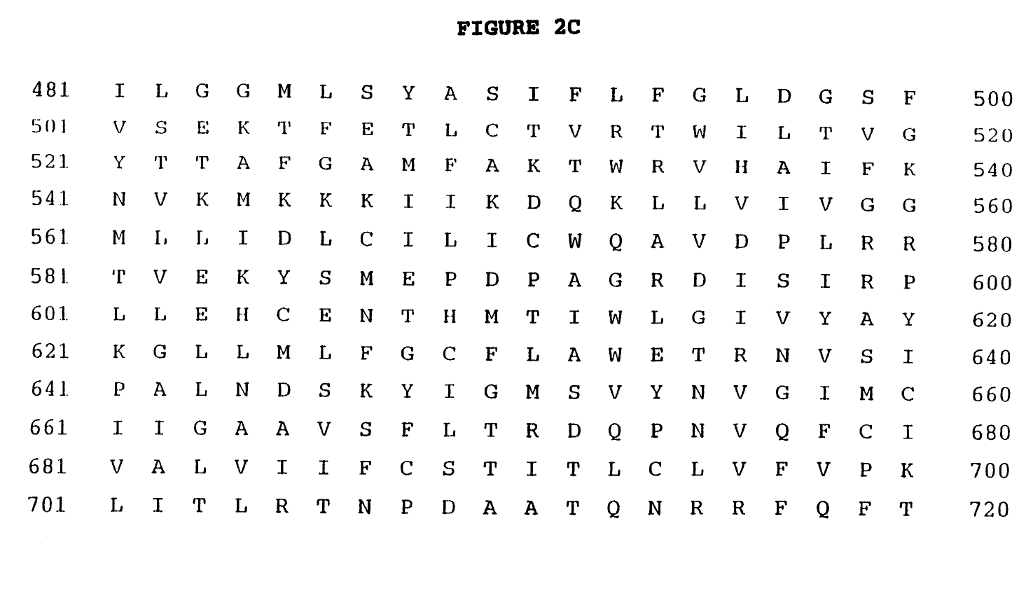

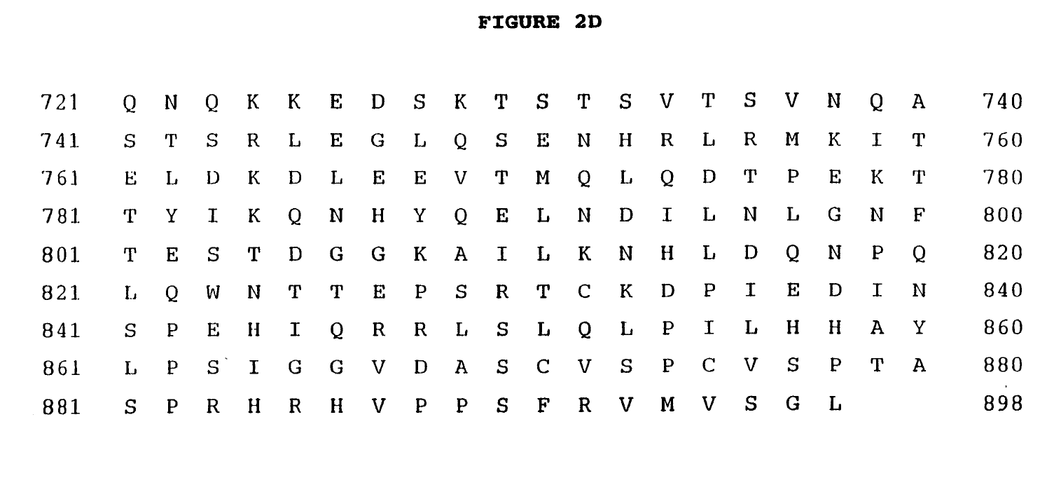

These results were used to obtain a full-length human clone TL-266, comprising both of the sequences identified by the BLAST search. Sequence analysis of clone TL-266 revealed a complete coding region for a novel protein. A search of the GenEMBL database indicated that the most similar sequence was that of GABA_{B}R1a , followed by G protein-coupled receptors (GPCRs) of the metabotropic receptor superfamily. The nucleotide and deduced amino acid sequence of TL-267 are shown in FIGS. 1 and 2, respectively. The nucleotide sequence of the coding region is 57% identical to the rat GABA_{B}R1a over a region of 1,686 bases. The longest open reading frame encodes an 898 amino acid protein with 38% amino acid identity to the rat GABA_{B}R1a polypeptide. Hydropathy plots of the predicted amino acid sequence reveal seven hydrophobic regions that may represent transmembrane domains (TMs, data not shown), typical of the G protein-coupled receptor superfamily. In the putative TM domains, GABA_{B}R2 exhibits 45% amino acid identity with the rat GABA_{B}R1a polypeptide. The amino terminus of TL-266 has amino acid homology to the bacterial periplasmic binding protein, a common feature of the metabotropic receptors (O'Hara et al. (1993) Neuron 11:41-52).

### Generation of rat GABA_{B}R2 PCR Product

Using PCR primers designed against the first and seventh transmembrane domains of the human GABA_{B}R2 sequence, BB257 and BB258, a 780 base pair fragment was amplified from rat hippocampus and rat cerebellum. Sequence from these bands displayed 90% nucleotide identity to the human GABA_{B}R2 gene. This level of homology is typical of a species homologue relationship in the GPCR superfamily.

### Construction and Screening of a Rat Hypothalamic cDNA Library

To obtain a full-length rat GABA_{B}R2 clone, pools of a rat hypothalamic cDNA library were screened by PCR using primers BB265 and BB266. A 440 base pair fragment was amplified from 44 out of 47 pools. Vector-anchored PCR was performed to identify pools with the largest insert size. One positive primary pool, I-47, was subdivided into 24 pools of 1000 individual clones and screened by vector-anchored PCR. Seven positive subpools were identified and one, I-47-4, was subdivided into 10 pools of 200 clones, plated onto agar plates, and screened by southern analysis. Four closely clustering colonies that appeared positive were rescreened individually by vector-anchored PCR. One positive colony, I-47-4-2, designated BO54, was amplified as a single rat GABA_{B}R2 clone. Since vector-anchored PCR revealed that BO54 was in the wrong orientation for expression, the insert was isolated by restriction digest and subcloned into the expression vector pEXJ. A transformant in the correct orientation was identified by vector-anchored PCR, and designated BO-55.

The nucleotide and deduced amino acid sequence of BO-55 are shown in FIGS. 3 and 4, respectively. BO-55 contains a 2.82 kB open reading frame and encodes a polypeptide of 940 amino acids. The nucleotide sequence of BO-55 is 89% identical to TL-267 in the coding region, with an overall amino acid identity of 98%. The proposed signal peptide cleavage site is between amino acids 40 and 41 (Nielsen et al., 1997).

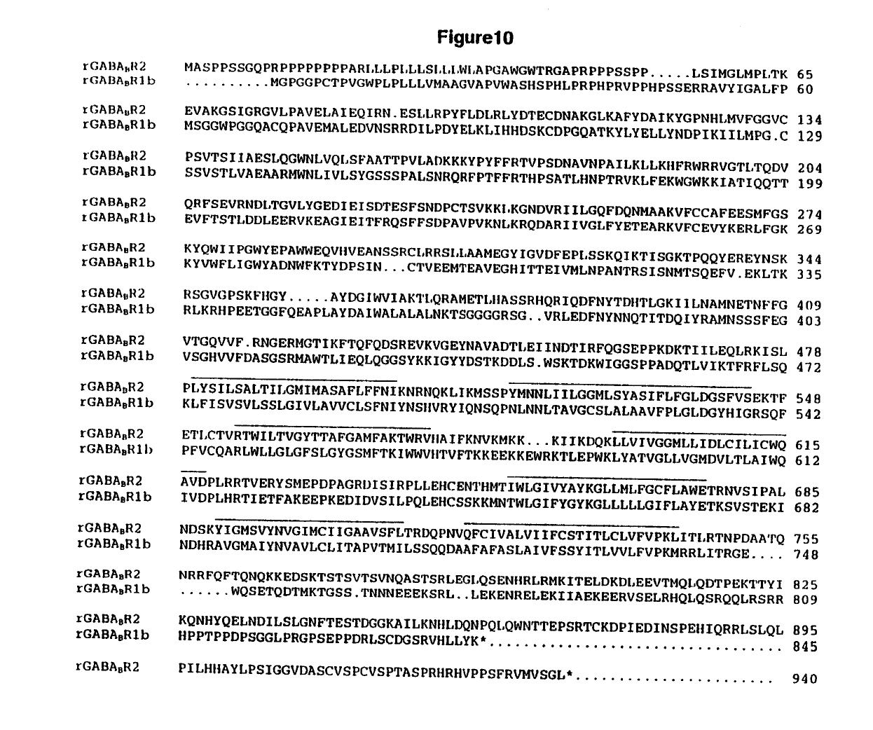

A BLAST search of GenEMBL indicated that this sequence was most closely related to GABA_{B}R1, displaying 35% and 41% amino acid identities overall and within the predicted transmembrane domains, respectively (FIG. 10). The structural similarity to GABA_{B}R1 indicated that this sequence encodes a novel polypeptide, which we refer to as GABA_{B}R2. The next most related sequences were other members of the mGluR family, with 21-24% overall amino acid identities. Like GABA_{B}R1 and other members of the mGluR family (O'Hara, P. J., et al., 1998), GABA_{B}R2 contains a large N-terminal extracellular domain having regions of homology to bacterial periplasmic binding proteins.

### Distribution of GABA_{B}R1 or GABA_{B}R2 in cDNA Libraries

Three cDNA libraries were screened by PCR with primers directed to transmembrane regions of either GABA_{B}R 1 or GABA_{B}R2. In a human hippocampal cDNA library both polypeptides were found in greater than 90% of the pools and in a rat hypothalamic cDNA library, again both polypeptides were found in greater than 90% of the pools. In addition, within each of these two libraries, the respective frequency of GABA_{B}R L and GABA_{B}R2 seems to be the same. However, in a rat spinal cord cDNA library, GABA_{B}R1 was found in 62.5% of the pools while GABA_{B}R2 was found in only 17.5% of the pools. Thus, while both polypeptide subtype appear to be present at high frequency in all three of the libraries, in the spinal cord library GABA_{B}R2 occurs at 3.6-fold lower frequency. These data point to the existence of an additional GABA_{B}-like peptide(s).

### Results of Localization Controls

The specificity of the hybridization of the GABA_{B}R2 probe was verified by performing in situ hybridization on transiently transfected HEK293 cells as described in Methods. The results indicate that hybridization to each of the individual GABA_{B}polypeptides was specific only to the HEK293 cells transfected with each respective cDNA. In addition, in situ hybridization on rat brain sections was performed using two hybridization probes targeted to different segments of the GABA_{B}R2 mRNA. In each case the pattern and intensity of labeling was identical in all regions of the rat CNS. Nonspecific hybridization signal was determined using the sense probes and was indistinguishable from background.

### Localization of GABA_{B}R2 mRNA in Rat CNS

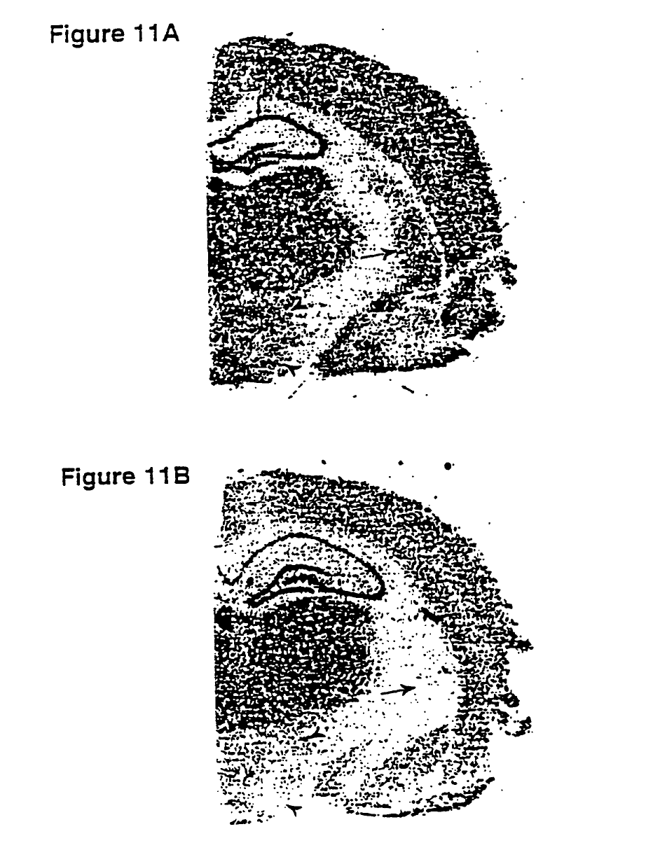

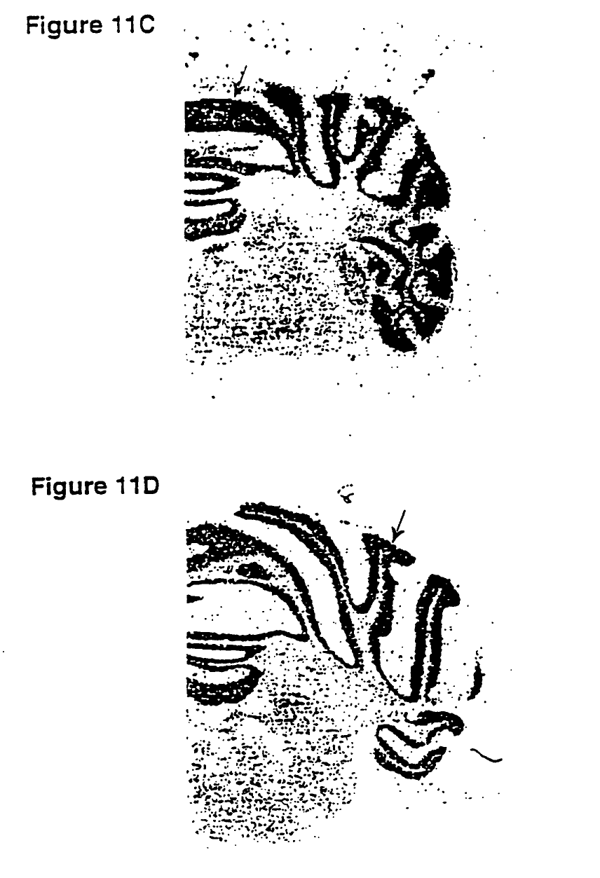

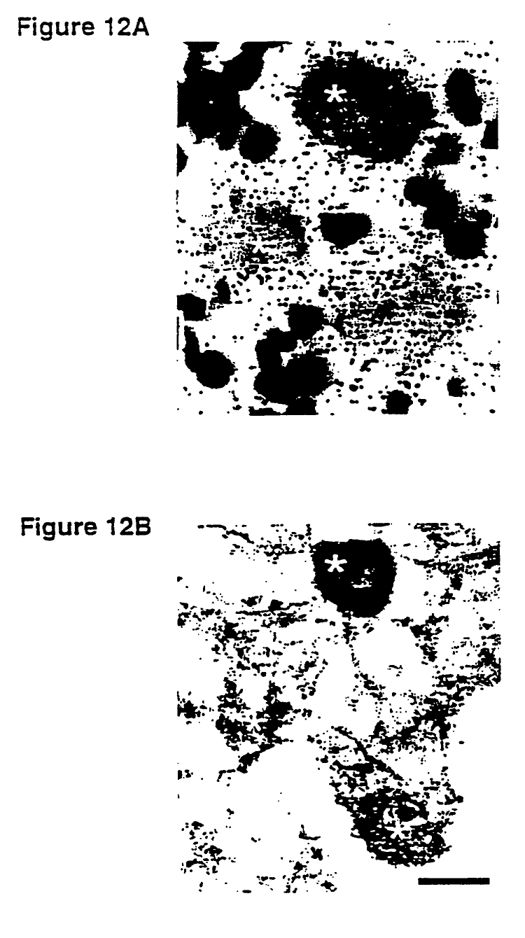

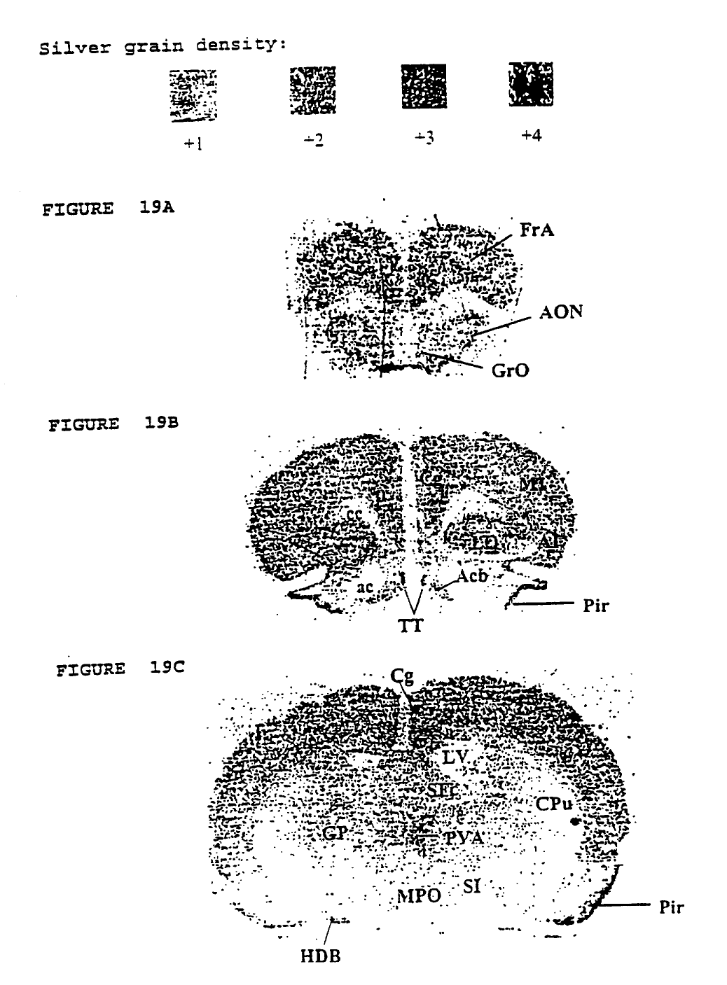

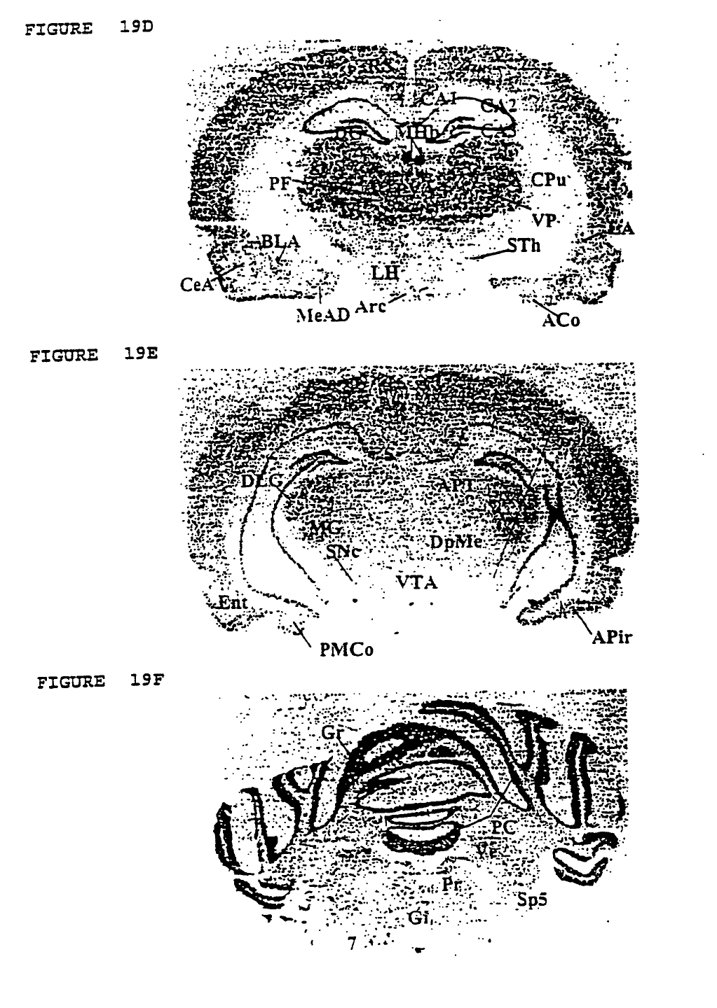

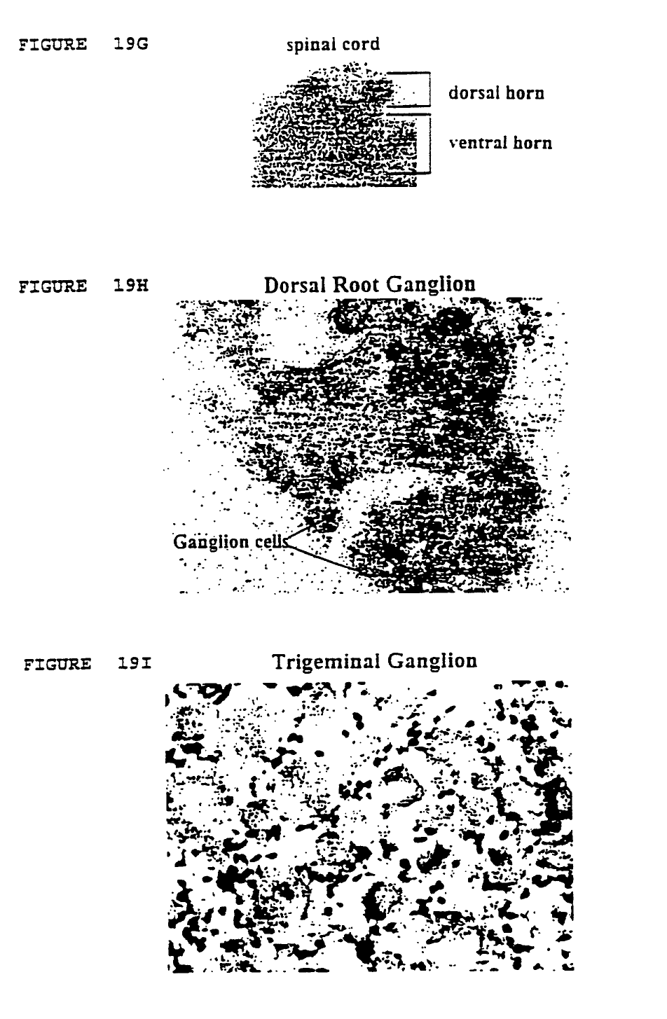

The anatomical distribution of GABA_{B}R2 mRNA in the rat brain was determined by in situ hybridization. By light microscopy the silver grains were determined to be distributed over neuronal profiles. The results suggest that the mRNA for GABA_{B}R2 is widely distributed throughout the rat CNS in addition to several sensory ganglia (FIGS. 19H-I) . However, expression levels in the brain were not uniform with several regions exhibiting higher levels of expression such as the medial habenula, CA3 region of the hippocampus, piriform cortex, and cerebellar Purkinje cells (FIGS. 19A-F). Moderate expression levels were observed in the ventral pallidum, septum, thalamus, CA1 region of the hippocampus, and geniculate nuclei (FIGS. 19C, D, E). Lower expression of GABA_{B}R2 mRNA was seen in the hypothalamus, mesencephalon, and several brainstem nuclei (FIGS. 19D, F). GABAergic neurons and terminals are likewise widely distributed in the CNS (Mugnaini, E., et al., 1985). and the distribution of the GABA_{B}R2 mRNA correlates well with the distribution of GABAergic neurons. One exception is the substantia nigra which contains high densities of GABAergic neurons, yet very low expression of GABA_{B}R2 mRNA. Additionally, the anatomical distribution of GABA_{B}R2 mRNA is in concordance with previous reports of the distribution of GABA_{B}binding sites obtained using [³H]baclofen (Gehlert, D. R., et al., 1985), and [³H]GABA (Bowery, N. J., et al., 1987). Furthermore, there was a high degree of similarity in the distribution and intensity of GABA_{B}R2 hybridization signal relative to those previously reported for GABA_{B}R1 (Bischoff, S., et al., 1997) (FIGS. 11, 12). Notable exceptions were the hypothalamus and caudate-putamen, where the expression of GABA_{B}R2 message appeared lower than that of GABA_{B}R1.

### Colocalization of GABA_{B}R2 and GABA_{B}R1b mRNAs in the Rat CNS

The results of the in situ hybridization studies using digoxygenin-labeled probe conjugated to alkaline phosphatase and the chromagen NBT/BCIP for the GABA_{B}R2 mRNA and an [³⁵S]dATP-labeled probe for the GABA_{B}R1b mRNA indicated that coexpression of the GABA_{B}R2 mRNA and GABA_{B}R1b mRNA did occur in vivo in neurons. In particular, colocalization was observed in cells of the medial habenula, hippocampus, and the cerebellar Purkinje cells. Likewise, there was evidence from the autoradiograms for potential overlapping distribution of the three known GABA_{B}mRNAs in the olfactory bulb, throughout the entire neocortex, several hypothalamic nuclei, numerous thalamic nuclei and brain stem nuclei. However, the Purkinje cells of the cerebellum contained message for only GABA_{B}R2 and GABA_{B}R1b and not the GABA_{B}R1a. Additionally, all three subtypes appear to be distributed throughout the gray matter of the spinal cord in all levels of the spinal cord.

The overlapping expression patterns of GABA_{B}R1 and GABA_{B}R2 transcripts in the brain suggested the possibility the polypeptides may be co-expressed in individual neurons and that both might be required for functional activity.

### Oocyte Expression

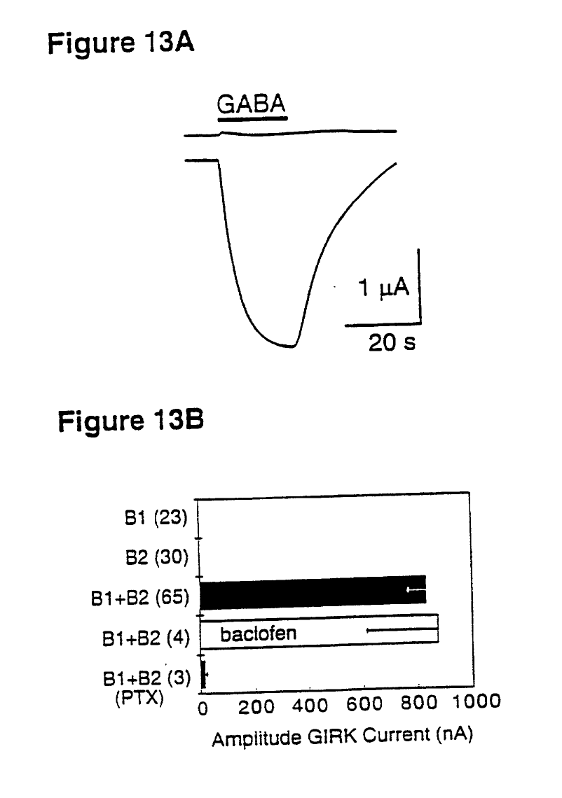

Postsynaptic inhibition of neurons by GABA_{B} receptor activation is caused by the opening of inwardly rectifying K⁺ channels (GIRK) (North, R. A., 1989; Andrade, R. et al., 1986; Gahwiler, B. H., et al., 1985; Luscher, C., et al., 1997). Oocytes expressing the combination of GABA_{B}R1b and GABA_{B}R2 mRNAs together with GIRKs elicited large currents in response to 30 μM GABA (Table 1a and 1b). (Subsequent to the compilation of the data in Table 1a, experiments were done to make Table 1b.) GABA and baclofen evoked sustained currents of similar magnitude (FIG. 13B). In contrast, oocytes expressing transcripts encoding either GABA_{b}R1a, GABA_{B}R1b, or GABA_{B}R 2 alone consistently failed to generate GIRK currents in response to high concentrations of GABA (1 mM), baclofen (1 mM) or 3-APMPA (100 μM). Others have reported similar results with GABA_{B}R1 (Kaupmann, K. et al., 1997a; Kaupmann, K., et al., 1997b).

TABLE 1a
Magnitude of GIRK currents stimulated by GABA
in oocytes and HEK-293 cells expressing GIRK1 and GIRK4
and various combinations of rat GABA_{B}R1 and rat GABA_{B}R2.

| | Oocytes | | | HEK-293 | | |
|---|---|---|---|---|---|---|
| | mean | | | mean | | |
| | (nA) | S.E.M. | (n) | (pA) | S.E.M. | (n*) |
| GABA_{B}R1a | 0 | 0 | (35) | — | — | — |
| GABA_{B}R1b | 0 | 0 | (15) | 5 | 3 | (3/26) |
| GABA_{B}R2 | 0 | 0 | (19) | 5 | 5 | (1/6) |
| GABA_{B}R1b+ | 1396 | 269 | (7) | 658 | 323 | (9/10) |
| GABA_{B}R2 | | | | | | |
| GABA_{B}R1b+ | 7 | 7 | (2) | — | — | — |
| GABA_{B}R2+ | | | | | | |
| PTX | | | | | | |

*number of cells responding/total number studied

TABLE 1b
Magnitude of GIRK currents stimulated by GABA
in oocytes and HEK-293 cells expressing GIRK1 and GIRK4
and various combinations of rat GABA_{B}R1 and rat GABA_{B}R2.

| | Oocytes | | | HEK-293 | | |
|---|---|---|---|---|---|---|
| | mean | | | mean | | |
| | (nA) | S.E.M. | (n) | (pA) | S.E.M. | (n*) |
| GABA_{B}R1a | 0 | 0 | (35) | — | — | — |
| GABA_{B}R1b | 0 | 0 | (23) | 5 | 3 | (5/26) |
| GABA_{B}R2 | 0.230 | .13 | (30) | .87 | .87 | (1/23) |
| GABA_{B}R1b+ | 832 | 65 | (65) | 470 | 71 | (70/81) |
| GABA_{B}R2 | | | | | | |
| GABA_{B}R1b+ | 16 | 9 | (3) | — | — | — |
| GABA_{B}R2+ | | | | | | |
| PTX | | | | | | |

*number of cells responding/total number studied

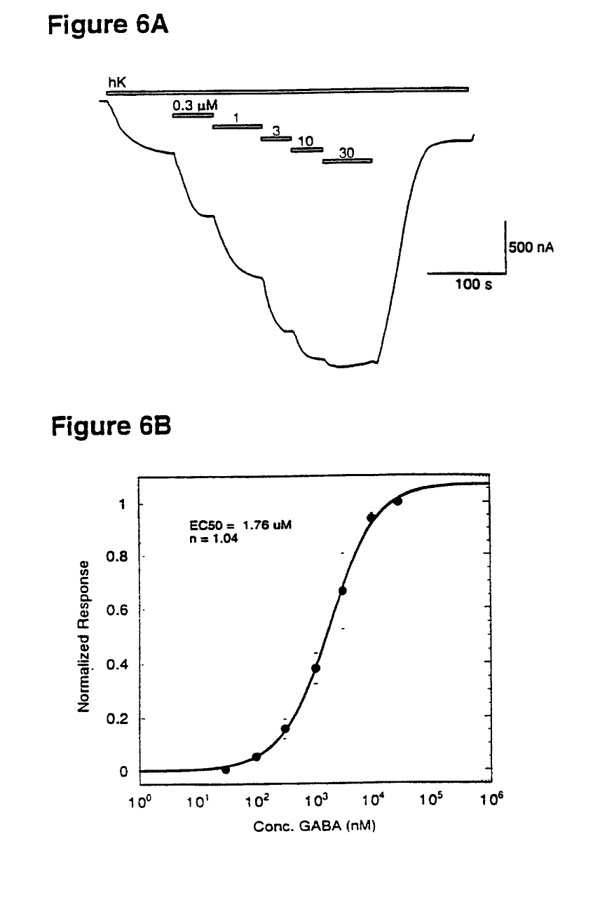

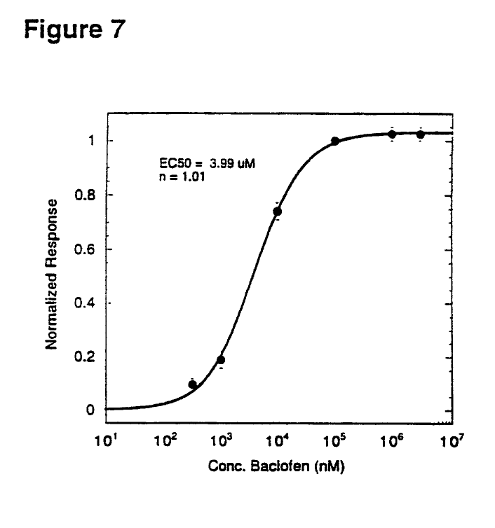

Currents stimulated by GABA in oocytes injected with both GABA_{B}R1b and GABA_{B}R2 mRNAs were completely blocked by the selective antagonist CGP55845 (1 μM) in a reversible fashion (data not shown). The potency of GABA and baclofen for eliciting GIRK currents was measured by performing steady-state cumulative concentration response assays on individual oocytes (FIG. 6A). Like K⁺ responses elicited by stimulation of native GABA_{B} receptors (Lacy et al. 1988; Misgeld et al. 1995), responses in oocytes did not desensitize and could be faithfully reproduced by multiple agonist applications on single oocytes. Stimulation of inward current was concentration dependent for both GABA and baclofen. The EC₅₀s, 1.76 μM for GABA and 3.99 μM for baclofen (FIG. 6B, FIG. 7), agreed closely with those reported in the literature for native receptors (Lacy et al. 1988; Misgeld et al. 1995). Concentration-effect curves for GABA were shifted to the right, in an apparently competitive manner, by well characterized GABA_{B}-selective antagonists (FIG. 15B). Based on additional experiments, the EC₅₀'s are 1.32 μM for GABA and 3.31 μM for baclofen. The results to date are summarized in Table 2. Antagonist affinity estimates (FIG. 15B, Table 2) were similar to values reported in previous electrophysiological studies using brain tissue (Bon, C., et al., 1996; Seabrook, G. R., et al., 1990), as well as to those obtained by measuring displacement of radioligand from cells expressing GABA_{B}R1 alone (Kaupmann, K., et al., 1997a) (Table 2).

TABLE 2
Agonist and antagonist pharmacology in cells
expressing GABA_{B}R1, GABA_{B}R2, or both.

| Protein Measurement Agonist | | | | | Antagonist | | |
|---|---|---|---|---|---|---|---|
| | | | Bacl | 3- | Phacl | CGP5 | CGP55 |
| | | GABA | ofen | APMPA | ofen | 4626 | 845 |
| GABA_{B} | pEC₅₀¹, | 5.88 ± | 5.48 | 7.29 ± | 3.80 | 7.48 ± | 8.60 ± |
| R1 + | pK₈² | 0.01 | ± | 0.02 | ± | 0.05 | 0.09 |
| GA | | | 0.05 | | 0.03⁴ | | |
| BA_{B}R2 | | | | | | | |
| GABA_{B} | pKᵢ³ | 4.6 | 4.3 | 5.2 | >3.0 | 8.95 | 8.7 |
| R1 | | | | | | | |

¹n = 6-8 oocytes except for GABA; n = 20 oocytes.
²Measured using GABA as agonist; n = 4-6 oocytes.
³Displacement of [³H]-CGP54626 from COS-7 cells expressing GABA_{B}R1; n = 3.4
⁴IC₅₀ using EC₅₀ concentration of GABA.

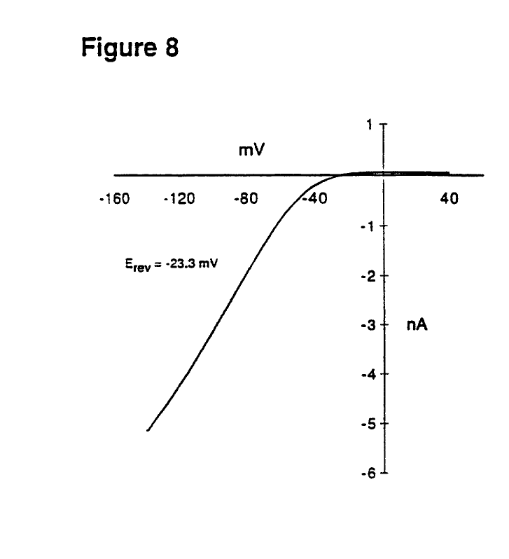

Evidence that GABA-induced currents were mediated by GIRK channels included: 1) dependency on elevated external K⁺, 2) strong inward rectification of the current-voltage (I/v) relation, 3) reversal potential (−23.3 mV) close to the predicted equilibrium potential for K⁺ (−23 mV), and 4) sensitivity to block by 100 μM Ba⁺⁺ (FIG. 8).

Three oocytes were injected with pertussis toxin (2 ng/oocyte) 6 h before voltage clamping. GABA-stimulated currents were abolished in these oocytes (Table 1a and 1b), suggesting that receptor activation of GIRKs was mediated by G-proteins Gᵢ or Gₒ. Analogous results have been obtained by others expressing D2 dopamine receptors with GIRKs in oocytes (Werner et al. 1996).

### GABA Responses in Co-transfected HEK-293 Cells

To verify that both gene products, GABA_{B}R1b and GABA_{B}R2, are also required for expression of functional GABA_{B} receptors in mammalian cells, voltage clamp recordings were obtained from HEK-293 cells transiently transfected with various combinations of each gene along with GIRKs. Cells transfected with a combination of GABA_{B}R1b (BO58) and GABA_{B}R2 (BO55) plus GIRKs consistently produced large K⁺ currents in response to 100 μM GABA (9 of 10 cells tested, Table 1a and 70 of 81 cells tested, Table 1b). Large amplitude currents were also observed when GABA_{B}R2 was paired with the GABA_{B}R1a splice variant (1046″ 247 pA; n=9). In contrast, cells transfected with only one of the GABA_{B} genes plus GIRKs responded either not at all or only very weakly to GABA (Table 1a and 1b). Small agonist-evoked currents (10-50 pA) were observed in 5 of 26 cells expressing GABA_{B}R1; similar weak currents were evoked in 1 of 23 cells expressing GABA_{B}R2.

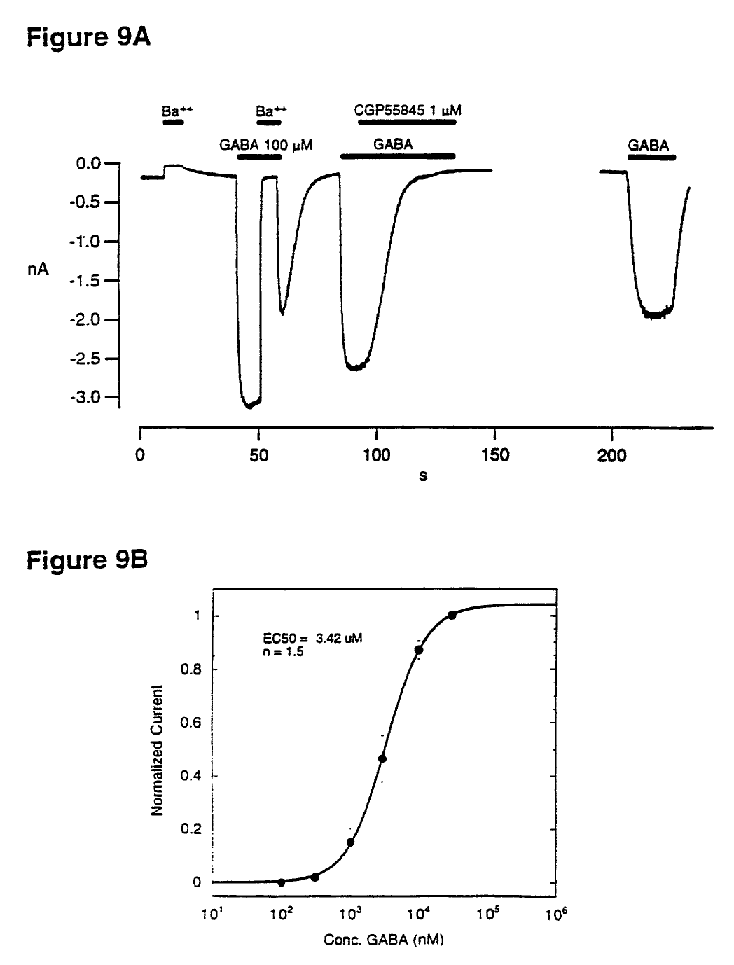

GABA-elicited currents in doubly transfected cells were completely blocked by 100 μM Ba⁺⁺ or the competitive antagonist CGP55845 at 1 μM (FIG. 9) . The EC₅₀ for GABA stimulation of GIRKs in HEK-293 cells was determined using similar methods as for oocytes. The EC₅₀, 3.42 μM, was comparable to that measured in oocytes (1.76 μM; further experiments gave 1.32 μM). Thus, whether in Xenopus oocytes or HEK-293 cells, the behavior of the GABA_{B} receptor is the same. Co-expression of both GABA_{B}R1b and GABA_{B}R2 is required to observe activation of the receptor by GABA.

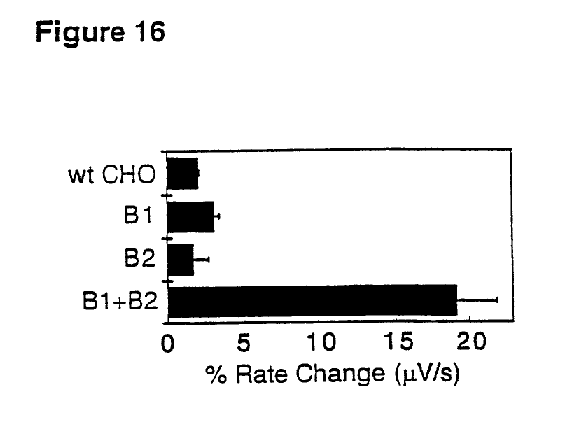

To determine if co-expressed GABA_{B}R1/R2 could mediate a cellular response in the absence of exogenously supplied GIRKs, we transiently co-transfected CHO cells with GABA_{B}R1 and GABA_{B}R2 and measured agonist-evoked extracellular acidification using a microphysiometer. Baclofen stimulated a 9-fold increase in acidification rate (FIG. 16) which was blocked by 100 nM CGP55845 and by pretreatment with PTX (not shown). This response was absent in cells expressing either protein alone. Since GIRK activity is undetectable in wild-type CHO cells (Krapivinsky, G., et al., 1995b) we conclude that GIRK expression is not a prerequisite for signal generation by GABA_{B}R1/R2.

### GABA_{B}R1/GABA_{B}R2 Signaling Through Chimeric G-proteins

Chimeric G-proteins have been used to “switch” the coupling pathway of a GPCR from one that normally inhibits adenylyl cyclase to one that activates phospholipase C (Conklin et al., 1993). With the aim of developing an assay based on Ca⁺⁺ or some other signal amenable to high throughput screening, we employed a Gα_{q/i3} chimera to obtain Ca⁺⁺-induced Cl⁻ responses in oocytes. Oocytes were injected with GABA_{B}R1 and GABA_{B}R2 mRNAs as previously described. 2-3 days later oocytes were injected again with 50 pg of Gα_{q/i3} mRNA and recorded under voltage clamp conditions. In response to GABA (0.1-1 mM) 88% of these oocytes produced rapidly desensitizing inward currents (454±92 nA; n=14) typical of those stimulated by receptors that normally couple to Gα_{q}. In contrast, oocytes injected with only the GABA_{B}R1/GABA_{B}R2 combination (n>100), or GABA_{B}R1 plus Gα_{q/i3} (n=4) failed to produce currents.

GABA_{B} agonists also resulted in concentration-dependent stimulation of phosphoinositide production in COS-7 cells transfected transiently with GABA_{B}R1, GABA_{B}R2, and the chimeric G-protein Gα_{q/z}. The concentration of agonist evoking 50% of its maximum response (EC₅₀) and fold stimulation over basal were: GABA (EC₅₀=1.8 μM; 2.4 fold); baclofen (1.7 μM; 1.8 fold); 3-aminopropylmethylphosphinic acid (EC₅₀=0.11 μM; 2.2 fold). These results indicate that G-protein chimeras, in particular Gα_{q/z} and Gα_{q/i3}, are useful for directing GABA_{B} receptor stimulation to a phosphoinositide- or Ca⁺⁺-based assay.

A comparison of the pharmacological properties of GABA_{B}R1 and GABA_{B}R2 using radioligand binding revealed that membranes from HEK293 or COS-7 cells expressing GABA_{B}R1, but not those expressing GABA_{B}R2, were labeled by the high affinity antagonist [³H]-CGP54626²¹ (Table 2), indicating that the polypeptides are pharmacologically distinct. Neither was labeled by the agonists [³H]-GABA or [³H]-baclofen. Furthermore, with the available ligands (GABA, baclofen, APMPA, phaclofen, CGP54626, CGP-55845 and SCH-50911) the binding profile of membranes from cells co-transfected with GABA_{B}R1/R2 was not different from those transfected with GABA_{B}R1 alone. The absence of detectable high affinity agonist binding to GABA_{B}R1/R2, as well as to GABA_{B}R1b, constitutes a notable distinction from the GABA_{B} binding profile in the CNS and may reflect the absence of an essential, as yet undefined G-protein or accessory protein.

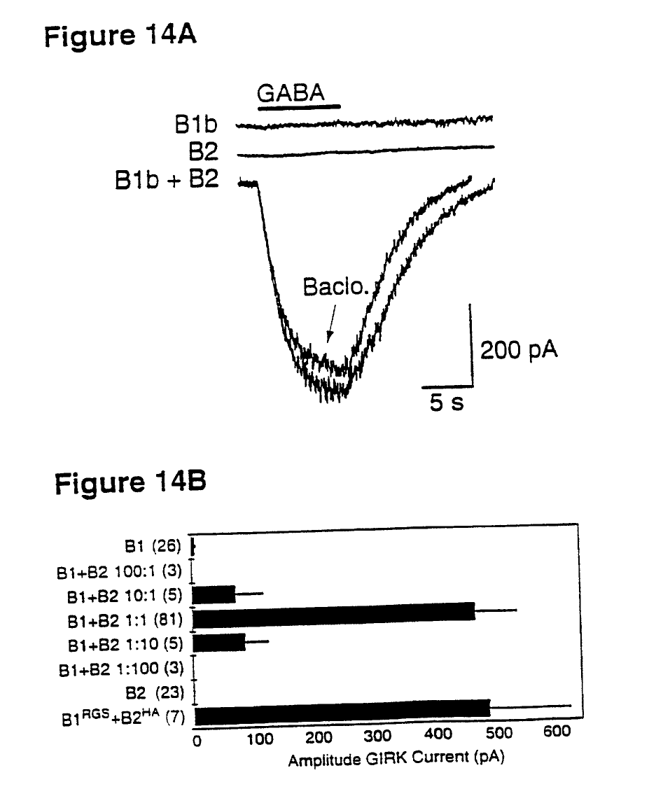

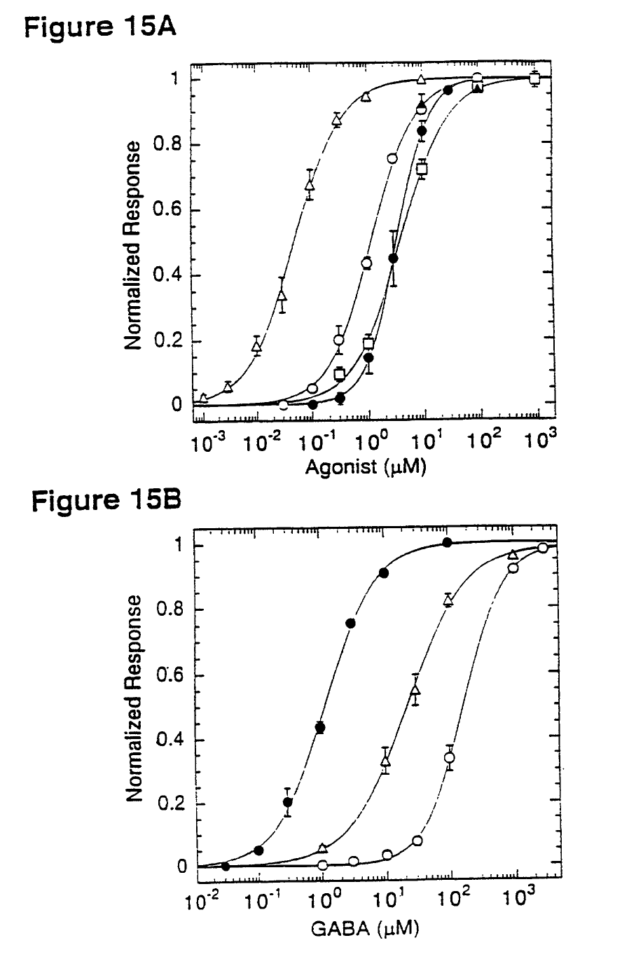

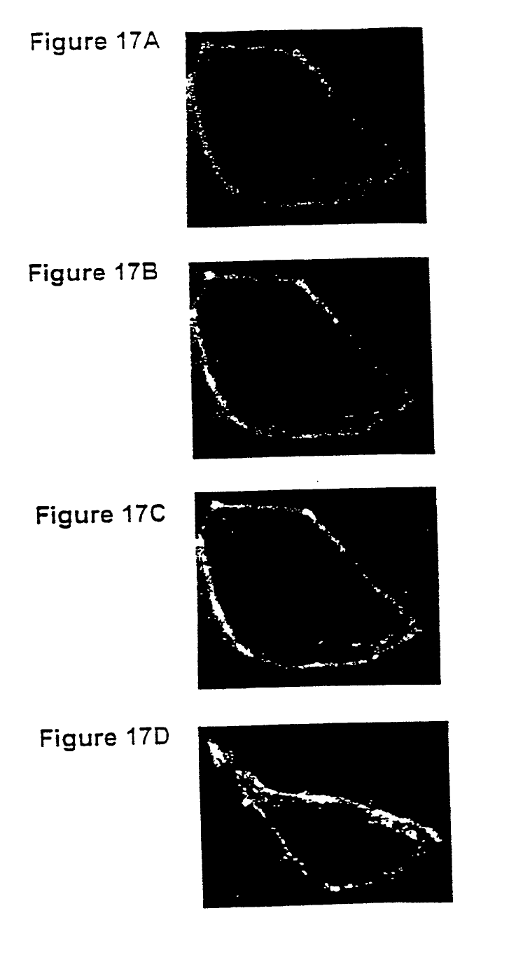

The molecular mechanism by which protein co-expression confers functional activity is unknown. We noted that varying the ratios of GABA_{B}R1/R2 cDNAs from 1/100 to 100/1 in HEK293 cells resulted in a symmetrical fall off in response amplitude (FIG. 14B). This suggests that a 1:1 protein stoichiometry may be critical, and caused us to postulate that the polypeptides are forming a heteromeric association. Biochemical evidence supports the idea that certain GPCRs can exist as homodimers (Hebert, T. E., et al., 1996; Cvejic, S., et al., 1997; Ciruela, F., et al., 1995; Avissar, S., et al., 1983; Romano, C., et al., 1996), but the functional significance of this has been largely unexplored (Hebert, T. E., et al., 1996; Wreggett, K. A., et al., 1995). The possibility of a physical association was investigated using epitope-tagged versions of GABA_{B}R1 (RGS6xH tag) and GABA_{B}R2 (HA tag). C-terminal modification did not appear to alter the function of either polypeptide; maximal current amplitudes (FIG. 14B) and EC₅₀ values for GABA (4.97 μM, n=5) were unchanged compared to HEK293 cells expressing the wild-type GABA_{B}R1/R2 receptor combination (3.42 μM, n=5). The subcellular distribution of epitope-tagged proteins was examined in transfected cells by fluorescence microscopy. When expressed individually, GABA_{B}R1^{RGS6xH} and GABA_{B}R2^{HA} were localized throughout the plasma membrane. Optical sectioning of antibody-labeled cells by confocal microscopy confirmed the membrane localization pattern, with less labeling in the cytoplasm and none in the nucleus. In co-transfected cells there was a striking overlap in the distribution of the two epitope tags (FIG. 17A-17C). Both proteins were prominently expressed on the plasma membrane. Furthermore, co-localization occurred within the cytoplasm, suggesting that GABA_{B}R1 and GABA_{B}R2 assemble in the endoplasmic reticulum. In contrast, the cellular distribution of an unrelated GPCR, NPY Y5, differed considerably from that of GABA_{B}R2 (FIG. 17D), suggesting specificity in the association of GABA_{B}R2 with GABA_{B}R1.

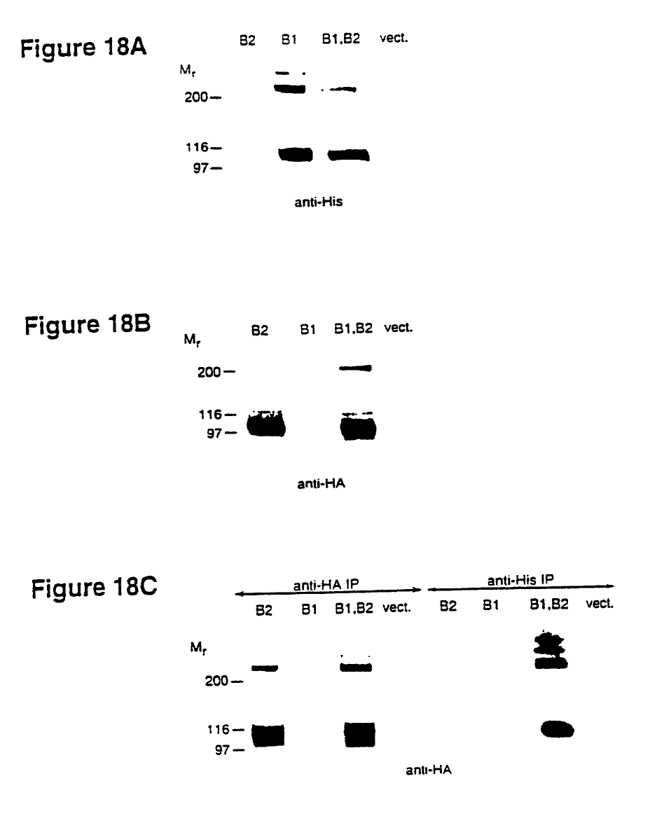

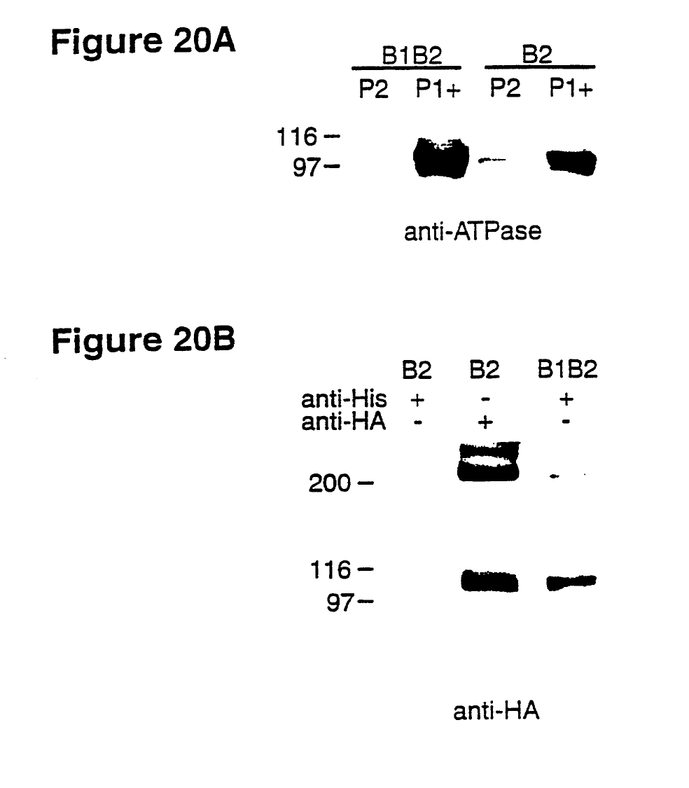

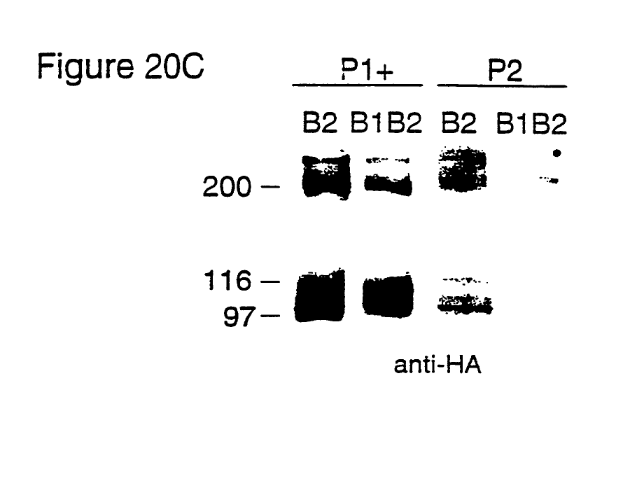

Western blots of whole cell extracts from cells expressing GABA_{B}R1^{RGS6xH}, GABA_{B}R2^{HA} or both, exhibited bands close to the predicted molecular weights of the two proteins (92 kD for GABA_{B}R1, 97 kD for GABA_{B}R2) and additional bands corresponding to the predicted molecular weights of receptor dimers (FIG. 18A,B). To determine if GABA_{B}R1 and GABA_{B}R 2 co-associate in a heteromeric complex, we immunoprecipitated solubilized material from cells expressing both polypeptides. GABA_{B}R2^{HA} was detected in material immunoprecipitated using either anti-His or anti-HA antibodies (FIG. 18). To determine if GABA_{B}R1b and GABA_{B}R2 co-associate in a heteromeric complex, we performed immunoprecipitations using membrane fractions enriched in plasma membrane as determined by the presence of Na⁺/K⁺ ATPase (FIG. 20A). In co-transfected cells only, GABA_{B}R2^{HA} was detected in material immunoprecipitated using antibodies specific for the GABA_{B}R1^{RGS6xH} protein (FIG. 20B). This result confirms that both GABA_{B}R1 and GABA_{B}R2 are correctly targeted to the plasma membrane of HEK293 cells, and that the two proteins exist in a heteromeric complex, perhaps as heterodimers, on the membrane surface.

### Responses Measured Using a Microphysiometer

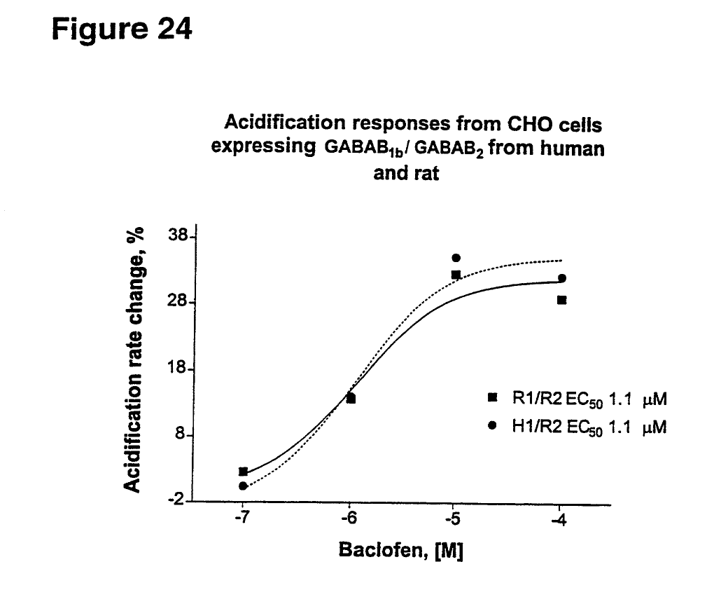

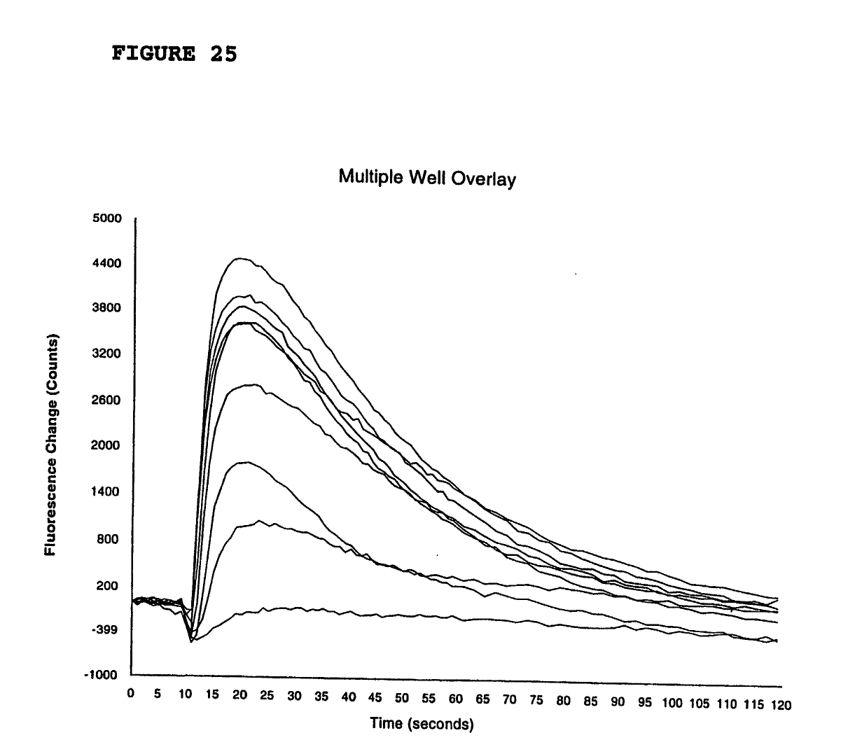

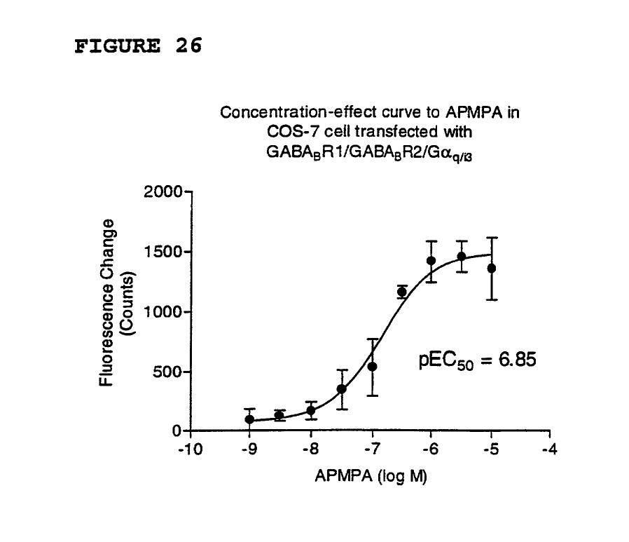

The results described so far using rat clones of GABA_{B}R1 and GABA_{B}R2 indicate that heterodimer formation is critical for the activation of GABA_{B} receptors by agonists. Co-expression experiments with human polypeptides suggest that the same phenomenon occurs for human homologues of GABA_{B}R1 and GABA_{B}R2. For example, using the microphysiometer we found that only when both rat GABA_{B}R1 and rat GABA_{B}R2 were co-expressed was there a dose-dependent acidification response to baclofen. In other experiments when human GABA_{B}R1 was substituted for rat GABA_{B}R1, and co-expressed with rat GABA_{B}R2, a similar concentration-dependent acidification response was observed (FIG. 24). The calculated EC₅₀ values for baclofen were indistinguishable between rat GABA_{B}R1/rat GABA_{B}R2 (1.09 μM) and human GABA_{B}R1/rat GABA_{B}R2 (1.08 μM). These data strongly support the notion that like the rat receptor, the human GABA_{B} receptor is formed by subunits composed of GABA_{B}R1 and GABA_{B}R2.

### GABA_{B} Mediated Calcium Responses Measured Using FLIPR

Confluent monolayers of COS-7 cells in 150 cm² flasks were transfected simultaneously with three individual plasmids containing (1) the human GABA_{B}R1 (5 ng), (2) the human GABA_{B}R2 (5 ng), and (3) the chimeric G-protein Gα_{q/13} (10 ng), using the DEAE/Dextran method (Lopata, et al., 1984). The cells were grown under controlled conditions for 24 h, at which time they were re-seeded into poly-D-lysine coated 96 well plates (Biocoat, VWR #62406-036) at a density of 50000 cells/well, and grown for another 24 h.

On the day of the assay, the medium was aspirated and 100 μl of loading buffer (Hank's buffer containing 20 mM HEPES, 1% FBS, 2.5 mM probenecid, and 4 μM Fluo-3 μM; F1241 Molecular Probes, Eugene, Oreg.) was added to each well. The cells were then incubated for 1 h at 37° C. in an atmosphere of 5% CO₂ The cells were then washed 3× with assay buffer (Hank's buffer containing 20 mM HEPES and 2.5 mM probenecid) at room temperature. The cells were left in a final volume of 150 assay buffer. To begin the assay, test compounds were added from drug plates in 50 μl volumes via the FLIPR instrument (Molecular Devices, Sunnyvale, Calif.) in a volume of 50 μl. Receptor-mediated increases in intracellular Ca⁺ concentration were recorded as flourescent units. After subtracting baseline fluorescence, responses above background were plotted versus agonist concentration to give final concentration effect curves. Concentration-effect curves were analyzed by non-linear regression analysis to derive agonist potencies.

The GABA_{B} agonist 3-APMPA evoked concentration dependent increases in intracellular Ca⁺ concentration in COS-7 cells transfected with GABA_{B}R1, GABA_{B}R2, and Gα_{q13}. The potency derived for 3-APMPA in this assay was 6.85 (pEC₅₀).

### Experimental Discussion

A gene has been cloned that shows 38% overall identity at the amino acid level with the recently cloned GABA_{B}R1 polypeptide. Important predicted features of the new gene product include 7 transmembrane spanning regions, and a large extracellular N-terminal domain. Like the GABA_{B}R1 gene product, GABA_{B}R2 by itself does not promote the activation of cellular effectors such as GIRKs. When co-expressed together, however, the two permit a GABA_{B} receptor phenotype that is quite similar to that found in the brain. The functional attributes of this reconstituted receptor include: 1) robust stimulation of a physiological effector (GIRKs), 2) EC₅₀s for GABA and baclofen in the same range as for GABA_{B} receptors previously studied in the CNS, 3) antagonism by the high affinity selective antagonist CGP55845, and 4) inhibition of receptor function by pertussis toxin. These attributes are not observed when either GABA_{B}R1 or GABA_{B}R2 is expressed alone.

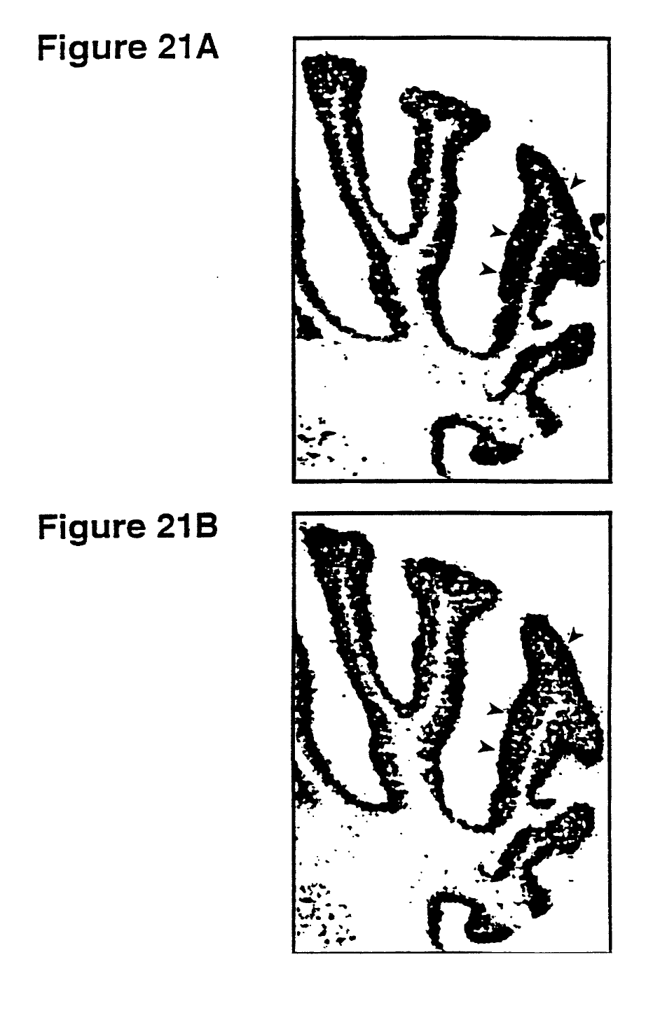

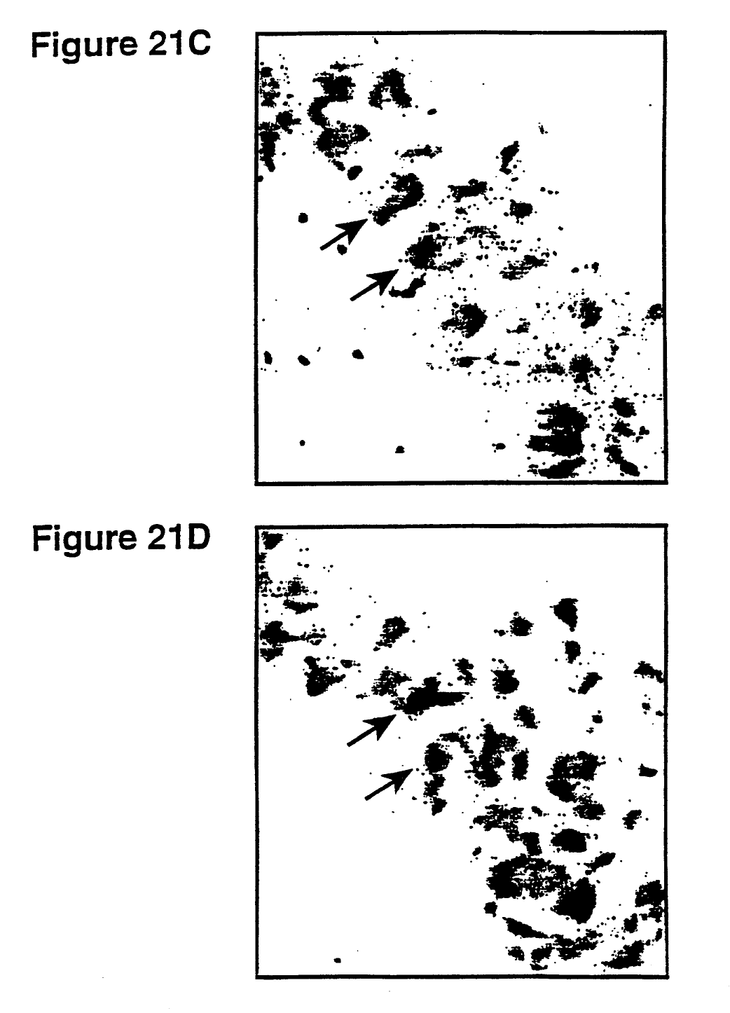

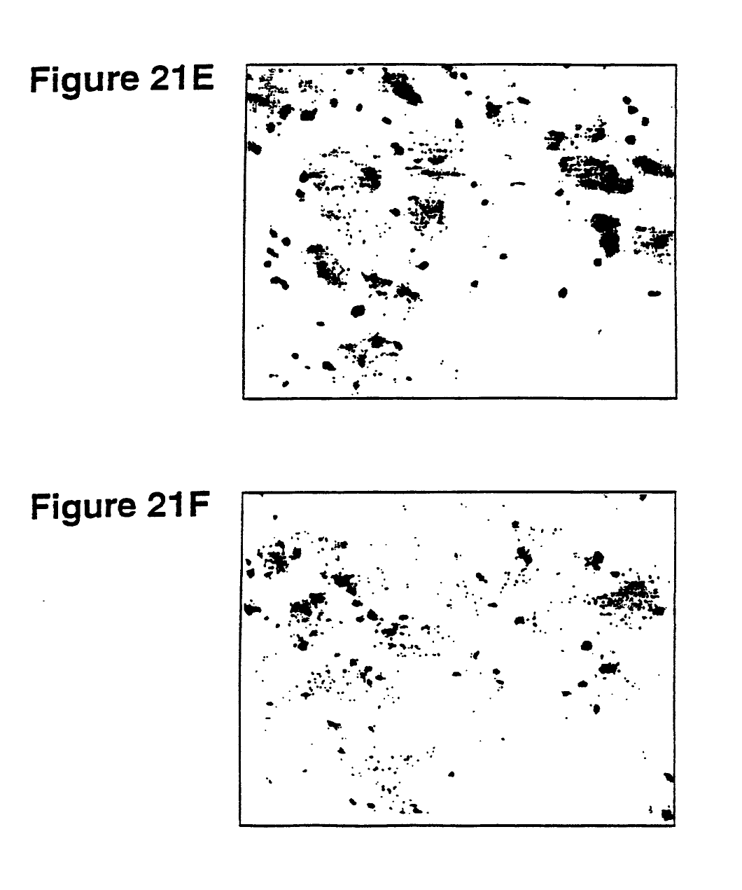

Our data indicate that GABA_{B}R1 and GABA_{B}R2 associate as subunits to produce a single pharmacologically and functionally defined receptor. Consistent with this view, double labeling in situ hybridization experiments provided evidence that GABA_{B}R1 and GABA_{B}R2 mRNAs are co-expressed in individual neurons and populations of neurons in several regions of the nervous system including hippocampal pyramidal cells (FIG. 21), cerebellar Purkinje cells (FIG. 12A,B) and sensory neurons in mesencephalic trigeminal nucleus (FIG. 21) and dorsal root ganglia. This co-localization pattern of GABA_{B}R1 and R2 transcripts predicts that GABA_{B} receptors on these cells are comprised of GABA_{B}R1/R2 heteromers. Other as yet unidentified GABA_{B} receptor homologues may associate elsewhere to produce novel subtypes. For example, the low level of expression of GABA_{B}R2 mRNA relative to GABA_{B}R1 in caudate putamen and hypothalamus (FIGS. 11A,B) raises the possibility that other GABA_{B} receptor homologues may associate with GABA_{B}R1 to produce novel subtypes in these regions. Conclusive evidence that functional GABA_{B} receptors exist in vivo as multimers will await immunofluorescence studies with specific antibodies.

The recent cloning of a family of accessory proteins that modify the binding and functional properties of a calcitonin-receptor-like receptor (McLarchie, et al., 1998) demonstrates that some 7-TM spanning proteins require additional unrelated proteins to reconstitute native GPCR activity. GABA_{B}R1 and GABA_{B}R2 are the first examples of 7-TM proteins for which activity is dependent on an interaction with another member within the same family of proteins. There will be considerable interest in whether other GPCRs are formed by heteromeric complexes of related 7-TM proteins. Many members of the superfamily of GPCRs, such as D₃, 5-HT₅, and olfactory receptors, do not function well in heterologous expression systems and may require related partners to generate native receptor function (Nimschinsky, et al., 1997). The growing list of receptors that have been reported to exist as homodimers (Ciruela, F., et al., 1995; Cvejic, S., et al., 1997; Hebert, T. E., et al, 1996; Romano, C., et al., 1996; Maggio, R., et al., 1996) points to the likelihood that both homomeric and heteromeric assemblies are more widespread among GPCRs than previously thought.

There are several possible explanations for why two genes are required for full function of the GABA_{B} receptor. One possible explanation is that the two gene products function together as a heterodimer having high affinity agonist and antagonist binding sites. Currently, there is no precedent for heterodimerization of GPCRs. There is evidence that certain GPCRs, for example the mGluR5 receptor, can form homodimers via cystine disulfide bridges in the N-terminal domain (Romano et al., 1996).

Significantly, synthetic peptides that inhibit homodimerization of beta2-adrenergic receptors also reduce agonist stimulation of adenylyl cyclase activity (Hebert et al., 1996). Useful parallels may be drawn from other classes of receptors where heterodimeric structures are well-known. For example, the NMDA (glutamate) receptor is comprised of two principal subunits, neither of which alone permits all of the native features of the receptor (see Wisden and Seeburg, 1993). GABA_{B} receptors may be comprised similarly of two (or more) peptide subunits, such as GABA_{B}R1 and GABA_{B}R2, that form a quaternary structure having appropriate binding sites for agonist and G-protein.

A role for GABA_{B}R2 in modulating sensory information is suggested by in situ hybridization histochemistry which revealed the expression of GABA_{B}R2 mRNA in relay nuclei of several sensory pathways. In the olfactory and visual pathways GABA_{B}R2 appears to be in a position to modulate excitatory glutamatergic projections from the olfactory bulb and retina GABA_{B}R2 mRNA was observed in the target regions of projection fibers from the main olfactory bulb, including the olfactory tubercle, piriform and entorhinal cortices and from the retina, for instance the superior colliculus (FIGS. 19A,B; Table 3).

The ability to modulate nociceptive information might be indicated not only by the presence of GABA_{B}R2 transcripts in somatic sensory neurons of the trigeminal and dorsal root ganglia (FIGS. 19H-I) but also by being present in the target regions of nociceptive primary afferent fibers, including the superficial layers of the spinal trigeminal nucleus and dorsal horn of the spinal cord (FIGS. 19F-G). Again, in each of these loci GABA_{B}R2 has been shown to be in a position to potentially modulate the influence of excitatory glutamatergic nociceptive primary afferents. In both ganglia, microscopic examination indicated that the hybridization signal did not appear to be restricted to any one size cell and was distributed evenly over small, medium and large ganglion cells. Thus, GABA_{B}R2 may be able to influence various sensory modalities. Expression levels appeared to be higher in the ganglion cells of the dorsal root with light to moderate expression in the trigeminal ganglia.

GABA_{B}R2 mRNA was likewise observed to be expressed in the vestibular nuclei which are target regions of inhibitory GABAergic Purkinje cells and also in the Purkinje cells themselves, suggesting that GABA_{B}R2 may be important in the mediation of planned movements (FIG. 19F).

Moderate expression of GABA_{B}R2 transcripts throughout the telencephalon indicate a potential modulatory role in the processing of somatosensory and limbic system (entorhinal cortex) information, in addition to modulating visual (parietal cortex) and auditory stimuli (temporal cortex) as well as cognition. Furthermore, modulation of patterns of integrated behaviors, such as defense, ingestion, aggression, reproduction and learning could also be attributed to this receptor owing to its expression in the amygdala (Table 3). The high levels of expression in the thalamus suggest a possible regulatory role in the transmission of somatosensory (nociceptive) information to the cortex and the exchange of information between the forebrain and midbrain limbic system (habenula). The presence of GABA_{B}R2 mRNA in the hypothalamus indicates a likely modulatory role in food intake, reproduction, the expression of emotion and possibly neuroendocrine regulation (FIG. 19D). A role in the mediation of memory acquisition and learning may be suggested by the presence of the GABA_{B}R2 transcript throughout all regions of the hippocampus and the entorhinal cortex (FIG. 19D).

TABLE 3
Distribution of rGABA_{B}R2, rGABA_{B}R1a, and GABA_{B}1b
mRNA in the rat CNS. The strength of the hybridization
signal for each of the respective mRNAs obtained in
various regions of the rat brain was graded as weak (+),
moderate (++), heavy (+++) or intense (++++) and is relative
to the individual polypeptides.

| Region | GABA_{B}R2 | GABA_{B}R1a* | GABA_{B}R1b* | Potential Application |
|---|---|---|---|---|
| Olfactory | | | | Modulation of |
| bulb | | | | olfactory sensation |
| internal | + | ++ | ++ | |
| granule | | | | |
| layer | | | | |
| glomerular | + | ++ | ++ | |
| layer | | | | |
| external | — | — | — | |
| plexiform | | | | |
| layer | | | | |
| mitral cell | — | + | ++ | |
| layer | | | | |
| anterior | ++ | ++ | ++ | |
| olfactory n | | | | |
| olfactory | + | ++ | +++ | |
| tubercle | | | | |
| Islands of | — | ++ | +++ | |
| Calleja | | | | |
| Telencephalon | | | | Sensory |
| | | | | integration |
| taenia | ++ | ++ | ++ | |
| tecta | | | | |
| frontal | ++ | ++ | ++ | |
| cortex | | | | |
| orbital | ++ | ++ | ++ | |
| cortex | | | | |
| agranular | +++ | ++ | ++ | |
| insular | | | | |
| cortex | | | | |
| cingulate | ++ | ++ | + | |
| cortex | | | | |
| retrosplenial | ++ | ++ | + | |
| cortex | | | | |
| parietal | ++ | ++ | ++ | Processing of |
| cortex | | | | visual stimuli |
| occipital | ++ | ++ | ++ | |
| cortex | | | | |
| temporal | ++ | ++ | ++ | Processing of |
| cortex | | | | auditory stimuli |
| perirhinal | ++ | ++ | | |
| cortex | | | | |
| entorhinal | ++ | ++ | ++ | Processing of |
| cortex | | | | visceral information |
| dorsal | ++ | ++ | ++ | |
| endopiriforn n | | | | |
| piriform | +++ | +++ | +++ | Integration/ |
| cortex | | | | transmission |
| | | | | of incoming |
| | | | | olfactory information |
| Basal Ganglia | | | | |
| accumbens n | + | ++ | ++ | Modulation of |
| | | | | dopaminergic |
| | | | | function |
| caudateputamen | + | + | ++ | Sensory/motor |
| | | | | integration |
| globus | + | — | + | |
| pallidus | | | | |
| Septum | | | | |
| medial | ++ | ++ | + | Cognitive |
| septum | | | | enhancement |
| | | | | via cholinergic |
| | | | | system |
| lateral | ++ | + | ++ | Modulation of |
| septum | | | | integration |
| | | | | of stimuli |
| | | | | associated |
| | | | | with |
| | | | | adaptation |
| septohippo- | + | + | +++ | |
| campal n | | | | |
| diagonal | ++ | ++ | ++ | |
| band n | | | | |
| ventral | ++ | + | + | |
| pallidum | | | | |
| Amygdala | | | | Anxiolytic |
| | | | | (activation - |
| | | | | reduction in |
| | | | | panic attacks) |
| | | | | appetite, depression |
| basolateral n | ++ | + | + | |
| medial | + | + | + | Olfactory |
| amygdaloid n | | | | amygdala |
| basomedial n | + | + | | |
| central n | +++ | — | + | |
| anterior | + | + | + | |
| cortical n | | | | |
| posteromedial | ++ | + | + | |
| cortical n | | | | |
| bed n stria | ++ | + | ++ | |
| terminals | | | | |
| zona incerta | + | + | + | |
| Hippocampus | | | | Memory |
| | | | | consolidation |
| | | | | and retention |
| CA1 , Ammon's | ++ | +++ | +++ | |
| horn | | | | |
| CA2 , Ammon's | ++++ | +++ | +++ | |
| horn | | | | |
| CA3 , Ammon's | ++++ | +++ | +++ | Facilitation |
| horn | | | | of LTP |
| subiculum | + | +++ | +++ | |
| parasubiculum | ++ | ++ | ++ | |
| presubiculum | ++ | ++ | ++ | |
| dentate gyrus | ++++ | +++ | ++ | |
| polymorph | +++ | +++ | ++ | |
| dentate gyrus | | | | |
| Hypothalamus | | | | |
| suprachiasm | + | ++ | ND | |
| atic n | | | | |
| median | + | + | ++ | Regulation of |
| preoptic | | | | gonadotropin |
| area | | | | secretion and |
| | | | | reproductive |
| | | | | behaviors |
| paraventricular n | + | ++ | ++ | Appetite/obesity |
| arcuate n | ++ | ++ | ++ | |
| anterior | + | + | | |
| hypoth, | | | | |
| post | | | | |
| lateral | + | + | ++ | |
| hypoth | | | | |
| ventromedial n | + | ++ | +++ | |
| periventricular n | + | + | + | |
| supraoptic n | + | ++ | + | Synthesis of |
| | | | | OXY and AVP |
| supramammillary n | ++ | ++ | ++ | Modulation of |
| | | | | hypothalamic |
| | | | | projections |
| | | | | to cortex |
| premammillary n | + | + | + | |
| medial | + | ++ | + | |
| mammillary n | | | | |
| Thalamus | | | | Analgesia/Mo |
| | | | | d-ulation of |
| | | | | sensory information |
| paraventricular n | ++ | + | ++ | Modulation of |
| | | | | motor and |
| | | | | behavioral |
| | | | | responses to |
| | | | | pain |
| centromedial n | ++ | + | ++ | Modulation of |
| | | | | motor and |
| | | | | behavioral |
| | | | | responses to |
| | | | | pain |
| paracentral n. | ++ | + | ++ | |
| parafascicular n | ++ | + | ++ | Modulation of |
| | | | | motor and |
| | | | | behavioral |
| | | | | responses to |
| | | | | pain |
| anterodorsal n | +++ | + | ++ | Modulation of |
| | | | | eye movement |
| laterodorsal n | +++ | + | ++ | |
| lateral | ++ | + | ++ | |
| posterior n | | | | |
| reuniens n | +++ | + | ++ | Modulation of |
| | | | | thalamic input to |
| | | | | ventral |
| | | | | hippocampus and |
| | | | | entorhinal ctx |
| rhomboid n | +++ | + | ++ | |
| medial | ++++ | + | ++++ | Anxiety/sleep |
| habenula | | | | disorders/ |
| | | | | analgesia in |
| | | | | chronic pain |
| lateral | + | + | +++ | |
| habenula | | | | |
| ventrolateral n | +++ | + | ++ | |
| ventromedial n | +++ | ++ | ++ | |
| ventral | +++ | + | ++ | |
| posterolateral n | | | | |
| reticular n | ++ | + | + | Alertness/sedation |
| lateral | ++ | + | ++ | Modulation of |
| geniculate n | | | | visual perception |
| medial | ++ | + | ++ | Modulation of |
| geniculate | | | | auditory system |
| subthalamic n | ++ | ++ | ++ | |
| Mesencephalon | | | | |
| superior | + | + | + | Modulation of |
| colliculus | | | | vision |
| inferior | + | + | + | |
| colliculus | | | | |
| central gray | + | + | + | Analgesia |
| dorsal raphe | + | ++ | + | |
| deep | + | + | + | |
| mesencephalic n | | | | |
| phalic n | | | | |
| oculomotor n | + | | | |
| pontine n | +++ | | ++ | |
| retrorubral field | + | | | |
| ventral | + | ++ | ++ | Modulation of |
| tegmental | | | | the |
| area | | | | integration |
| | | | | of motor |
| | | | | behavior and |
| | | | | adaptive |
| | | | | responses |
| substantia | + | + | + | Motor control |
| nigra, | | | | |
| reticular | | | | |
| substantia | ++ | ++ | ++ | |
| nigra, | | | | |
| compact | | | | |
| interpedunc ular n | ++ | ND | ND | Analgesia |
| Myelencephalon | | | | Analgesia |
| raphe magnus | ++ | | ++ | |
| raphe pallidus | + | ++ | ND | |
| principal trigeminal | + | + | | |
| spinal trigeminal n | + | + | + | |
| pontine reticular n | ++ | + | ++ | |
| parvicellular | + | ++ | ++ | |
| reticular n | | | | |
| locus | ++ | ++ | ++ | Modulation of |
| coeruleus | | | | NA transmission |
| parabrachial n | + | ++ | + | Modulation of |
| | | | | visceral sensory |
| | | | | information |
| vestibular n | + | ++ | + | Maintenance of |
| | | | | balance and |
| | | | | equilibrium |
| gigantocel | + | ++ | ++ | Inhibition |
| l - ular | | | | and |
| reticular n | | | | disinhibition |
| | | | | of brainstem |
| prepositus | + | +++ | ++ | Position and |
| hypoglossal n | | | | movement of |
| | | | | the eyes/ |
| | | | | Modulation of |
| | | | | arterial |
| | | | | pressure and |
| | | | | heart rate |
| ventral cochlear n | ++ | + | ND | |
| n soltary tract | ++ | | | Hypertension |
| A5 Noradrenaline cells | + | ND | ND | |
| facial n(7) | + | ++ | + | |
| Cerebellum | | | | Motor |
| | | | | coordination, Autism |
| granule | + | + | + | |
| cell layer | | | | |
| Purkinje | ++ | — | ++ | |
| cells | | | | |
| Spinal cord | | | | Analgesia |
| dorsal horn | + | ++ | + | |
| ventral | + | ++ | + | |
| horn | | | | |
| trigeminal | ++ | +++ | + | Nociception |
| ganglion | | | | |
| dorsal root | ++++ | +++ | ND | Nociception |
| ganglion | | | | |

ND = not determined
*Bischoff S et al.

### List of Abbreviations

7 facial n

ac anterior commisure

Acb accumbens n

ACo anterior cortical amygdaloid n

AI agranular insular cortex

AON anterior olfactory n

APir amygdalopiriform transition area

APT anterior pretectal n

Arc arcuate hypothalamic n

BLA basolateral amygdaloid n

CA1-3 Fields of Ammon's horn

cc corpus callosum

Cg cingulate cortex

CeA central amygdaloid n

CPU caudate-putamen

DG dentate gyrus

DLG dorsal lateral geniculate n

DpMe deep mesencephalic n

Ent entorhinal cortex

Gi gigantocellular reticular n

Gr granule cll layer, cerebellum

GrO granule layer olf. bulb

FrA frontal association cortex

GP globus pallidus

HDB horizontal diagonal band

LA lateral amygdaloid n

LH lateral hypothalamus

LO lateral orbital cortex

LV lateral ventricle

Ml primary motor cortex

MeAD medial amygdaloid n, anterodorsal

MG medial geniculate

MHb medial habenular n

MPO medial preoptic n

PC Purkinje cell layer of the cerebellum

PF parafascicular n

Pir piriform cortex

PMCO posteromedial cortical amygdaloid n

Pr prepositus n

PVA paraventricular thalamic n

RS retrosplenial cortex

S subiculum

SFi septofimbrial n

SI substantia innominata

SNc substantia nigra,compact

STh subthalamic n

Sp5 spinal trigeminal n

TT tenia tecta

Ve vestibular n

VTA ventral tegmental area

### Potential Therapeutic Application for GABA_{B} Agonists and Antagonists

### Agonists

### Antinociception

A potential GABA_{B} agonist application may in antinociception. The inhibitory effects of GABA and GABA_{B} agonists are thought to be predominantly a presynaptic mechanism on excitation-induced impulses in high threshold Ad and C fibers on primary afferents. This effect can be blocked by GABA_{B} antagonists (Hao,J- H., et al., 1994). Baclofen's spinal cord analgesic effects have been well documented in the rat, though it has not been as effective in human. However, baclofen has been successful in the treatment of trigeminal neuralgia in human.

The localization of the GABA_{B}R2 mRNA in the superficial layers of the spinal cord dorsal horn, the termination site for primary afferents, as well as their cells of origin in the dorsal root and trigeminal ganglia position the GABA_{B}R1/R2 receptor appropriately for mediating the agonist effects.

### Drug Addiction

It has been suggested that GABA agonists may have some potential in the treatment of cocaine addiction. A role for the action of psychostimulants in the mesoaccumbens dopamine system is well established. The ventral pallidum receives a GABAergic projection from the nucleus accumbens and both regions contain GABA_{B}R2 transcripts. GABA receptors were shown to have an inhibitory effect on dopamine release in the ventral pallidum. Phaclofen acting at these receptors resulted in increased dopamine release and baclofen was shown to attenuate the reinforcing effects of cocaine. (Roberts, D. C. S., et al.,1996; Morgan,A. E. et al.)

### Micturition

There is a potential application for GABA_{B} agonists in the treatment of bladder dysfunction. Baclofen has been used in the treatment of detrussor hyperreflexia through inhibition of contractile responses. In addition to a peripheral site of action for GABA_{B} agonists, there is also the possibility for a central site. The pontine micturition center in the brainstem is involved in mediating the spinal reflex pathway, via Onuf's nucleus in the sacral spinal cord. Support for possible application of GABA_{B} agonists in the treatment of bladder dysfunction may be augmented by presence of GABA_{B}R2 mRNA in the various nuclei involved in the control of the lower urinary tract function.

### Antagonists

### Memory Enhancement—Alzheimer's Disease

GABA_{B} antagonists may have a potential application in the treatment of Alzheimer's Disease. The blockade of GABA_{B} receptors might lead to signal amplification and improvement in cognitive functions resulting from an increased excitability of cortical neurons via amplification of the acetycholine signal. Additionally, memory may be enhanced by GABA_{B} antagonists which have been shown to suppress late IPSPs, thus facilitating long-term potentiation in the hippocampus (see Table 3).

To support this idea, CGP36742, a GABA_{B} antagonist, has been shown to improve learning performance in aged rats as well as the performance of rhesus monkeys in conditioned spatial color task. (Mondadori, C. et al., 1993). The significance of the GABA_{B}R1/R2 receptor in cognitive functioning might be indicated by the presence of GABA_{B}R2 mRNA in the cerebral cortex and its codistribution in the ventral forebrain with cortically projecting cholinergic neurons as well as its localization in the pyramidal cells in all regions of Ammon's horn and dentate gyrus in the hippocampus.

### References

Andrade, R., Malenka, R. C., Nicoll, R. A. A G protein couples serotonin and GABA_{B} receptors to the same channels in hippocampus. (1986) Science 234:1261-1265.

Aruffo, A. and Seed, B. (1987). Molecular cloning of a CD28 cDNA by a high efficiency COS Cell expression system. Proc. Natl. Acad. Sci. USA, 84, 8573-8577.

Avissar, S., Amitai, G. & Sokolovsky, M. (1983) Oligomeric structure of muscarinic receptors is shown by phocoaff inity labeling: subunit assembly may explain high- and low-affinity agonist states. Proc. Natl. Acad. Sci. U.S.A. 80:156-159.

Bischoff, S., Leonhard, N., Reyman, N, Schuler, V., Kaupmann, K., Bettler, B. (1997) Distribution of the GABA-BR1 mRNA in rat brain: comparison with the GABA-B binding sites. Soc . Neurosci. Abstr. 23:954 (Abstract).

Bolser, D. C., Aziz, S. M., DeGennaro, F. C., Kreutner, W., Egan, R. W., Siegel, M. .I, Chapman, R. W. (1993) Antitussive effects of GABA_{B} agonists in the cat and guinea-pig. Br J Pharmacol 110:491-495.

Bon, C., Galvan, M. Electrophysiological actions of GABAB agonists and antagonists in rat dorso-lateral septal neurones in vitro. (1996) Br.J.Pharmacol. 118:961-967.

Bonanno, G., Raiteri, M. (1992). Functional evidence for multiple gamma-aminobutyric acid B receptor subtypes in the rat cerebral cortex. J. Pharmacol. Exp. Ther. 262:114-118.

Bonanno, G., Raiteri, M. (1993). Multiple GABAB receptors. Trends Pharmacol. Sci. 14:259-261.

Bowery, N. G. (1993). GABA_{B} receptor pharmacology. Ann. Rev. Pharmacol. Toxicol. 33:109-47:109-147.

Bowery, N. G., Hudson, A. L., Price, G. W. (1987). GABAA and GABAB receptor site distribution in the rat central nervous system. Neuroscience 20:365-383.

Bowery, N. G., Knott, C., Moratalla, R., Pratt, G. D. (1990). GABAB receptors and their heterogeneity. Adv. Biochem. Psychopharmacol. 46:127-39:127-139.

Chapman, R. W., Hey, J. A., Rizzo, C. A., Bolser, D. C. (1993). GABAB receptors in the lung. Trends Pharmacol. Sci. 14:26-29.

Chu, D. C., Albin, R. L., Young, A. B., Penney, J. B. Distribution and kinetics of GABA_{B}binding sites in rat central nervous system: a quantitative autoradiographic study. (1990) Neuroscience 34(2):341-57.

Ciruela, F., Casado, V., Mallol, J., Canela, E. I., Lluis, C., Franco, R. (1995) Immunological identification of Al adenosine receptors in brain cortex. J.Neurosci.Res. 42:818-828.

Conklin, B. R., Farfel, Z., Lustig, K. D., Julius, D. & Bourne, H. R. (1993). Substitution of three amino acids switches receptor specificity of Gq-alpha to that of Gi-alpha. Nature 363:274-276.

Cvejic, S., Devi, L. A. Dimerization of the delta opioid receptor: implication for a role in receptor internalization. (1997) J.Biol.Chem. 272:26959-26964.

Dirig, D. M., Yaksh, T. L. (1995) Intrathecal baclofen and muscimol, but not midazolam, are antinociceptive using the rat-formalin model. J Pharmacol Exp Ther 275:219-227.

Durkin, M. M., Smith, K. E., Borden, L. A., Weinshank, R. L., Branchek, T. A. and Gustafson, E. L. Localization of messenger RNAs encoding three GABA transporters in rat brain: an in situ hybridization study. (1995) Brain Res. Mol. Brain Res. 33:7-21.

Erdo, S. L., Riesz, M., Karpati, E., Szporny, L. (1984). GABAB receptor-mediated stimulation of the contractility of isolated rabbit oviduct. Eur. J. Pharmacol. 99:333-336.

Gahwiler, B. H., Brown, D. A. GABAB -receptor-activated K+ current in voltage-clamped CA3 pyramidal cells in hippocampal cultures. (1985) Proc.Natl.Acad.Sci.U.S.A. 82:1558-1562.

Gehlert, D. R., Yamamura, H. I., and Wamsley, J. K. Υ-Aminobutyric acid_{B} receptors in the rat brain: quantitative autoradiographic localization using [³H] (−)baclofen. (1985) Neuroscience Lett. 56:183-188.

Giotti, A., Luzzi, S., Spagnesi, S., Zilletti, L. (1983). GABAA and GABAB receptor-mediated effects in guinea-pig ileum. Br. J. Pharmacol. 78:469-478.

Graham J (1984) Isolation of subcellular organelles and membranes. In: Centrifugation (The Practical Approach Series) (Rickwood D ed), pp 161-182. Oxford: IRL Press Ltd.

Gübler, U., Hoffman, B. J. (1983). A simple and very efficient method for generating cDNA libraries. Gene, 25, 263-269.

Hao,J -H., Xiao-Jun,X., Wiesenfeld-Hallin, Z. (1994) Neurosci. Lett. 182, 299-302.

Hebert, T. E., Moffett, S., Morello, J. P., Loisel, T. P., Bichet, D. G., Barret, C., Bouvier, M. (1996) A peptide derived from a beta2-adrenergic receptor transmembrane domain inhibits both receptor dimerization and activation. J Biol Chem 271:16384-16392.

Hills, J. M., Dingsdale, R. A., Parsons, M. E., Dolle, R. E., Howson, W. (1989). 3-Aminopropylphosphinic acid—a potent, selective GABAB receptor agonist in the guinea-pig ileum and rat anococcygeus muscle. Br. J. Pharmacol. 97:1292-1296.

Jones, K. A., Wilding, T. J., Huettner, J. E., Costa, A -M. (1997) Desensitization of kainate receptors by kainate, glutamate and diastereomers of 4-methylglutamate. Neuropharmacol 36:853-863.

Kaupmann, K., Huggel, K., Heid, J., Flor, P. J., Bischoff, S., Micke,l S. J., McMaster, G., Angst, C., Bittiger, H., Froestl, W., Bettler, B. (1997a) Expression cloning of GABA_{B} receptors uncovers similarity to metabotropic glutamate receptors. Nature 386:239-246.

Kaupmann, K., Mosbacher, J., Schuler, V., Flor, P. I., Froestl, W., Bittiger, H., Sommer, B., Bettler, B. (1997b) Structure, pharmacology and chromosomal localization of GABA-B receptors. Soc. Neurosci. Abstr. 23:954(Abstract).

Kofuji, P., Hofer, M., Millen, K. J., Millonig, J. H., Davidson, N., Lester, H. A., Hatten, M. E. (1996) Functional analysis of the weaver mutant GIRK2 K+ channel and rescue of weaver granule cells. Neuron 16:941-952.

Kozak, M. (1989). The scanning model for translation: an update. J. Cell Biol. 108: 229-241.

Kozak, M. (1991). Structural features in eukaryotic mRNAs that modulate the initiation of translation. J. Biol. Chem. 266: 19867-19870.

Krapivinsky, G., Krapivinsky,L., Wickman, K., Clapham, D. E. (1995a) G beta gamma binds directly to the G protein-gated K+ channel, IKACh. J Biol Chem 270:29059-29062.

Krapivinsky, G., Gordon, E. A., Wickman, K., Velimirovic, B., Krapivinsky, L., Clapham, D. E. The G-protein-gated atrial K channel I_{KACh} is a heteromultimer of two inwardly rectifying K channel proteins. (1995b)_Nature 374:135-141.

Lacey, M. G., Mercuri, N. B., North, R. A. (1988) On the potassium conductance increase activated by GABAB and dopamine D2 receptors in rat substantia nigra neurones. J Physiol (Lond) 401:437-53:437-453.

Leyson, J. F., Martin, B. F., Sporer, A. (1980) Baclofen in the treatment of detrusor-sphincter dyssynergia in spinal cord injury patients. J. Urol. 124:82-84.

Lopata, M. A., Cleveland, D. W., Sollner-Webb, B. High level transient expression of a chloramphenicol acetyl transferase gene by DEAE-dextran mediated DNA transfection coupled with a dimethyl sulfoxide or glycerol shock treatment. (1984) Nucleic Acids Res. 12: 5707-5717.

Luscher, C., Jan, L. Y., Stoffel, M., Malenka, R. C., Nicoll, R. A. G protein-coupled inwardly rectifying K+ channels (GIRKS) mediate postsynaptic but not presynaptic transmitter actions in hippocampal neurons [Published erratum appears in Neuron 1997 Oct. 19(4): following 945]. (1997)₁₃Neuron 19, 687-695.

Luzzi, S., Franchi-Micheli, S., Folco, G., Rossoni, G., Ciuffi, M., Zilletti, L. (1987) Effect of baclofen on different models of bronchial hyperreactivity in the guinea-pig. Agents Actions 20:307-309.

Maggio, R., Barbier, P., Fornai, F., Corsini, G. U. Functional role of the third cytoplasmic loop in muscarinic receptor dimerization. (1996) J.Biol.Chem. 271:31055-31060.

Malcangio, M., Ghelardini, C., Giotti, A., Malmberg-Aiello, P. , Partolini, A. (1991) CGP 35348, a new GABAB antagonist, prevents antinociception and muscle-relaxant effect induced by baclofen. Br. J. Pharmacol. 103:1303-1308.

McCormick, M. (1987). Sib selection. Meth. Enzymol. 151:445-449.

McLatchie, L. M., Fraser, N. J., Main, M. J., et al. RAMPs regulate the transport and ligand specificity of the calcitonin-receptor-like receptor. (1998) Nature 393:333-339.

Misgeld, U., Bijak, M., Jarolimek, W. (1995). A physiological role for GABAB receptors and the effects of baclofen in the mammalian central nervous system. Prog. Neurobiol. 46:423-462.

Mondadori, C., Jaekel, J., Preiswerk, G. CGP36742: The First Orally Active GABA_{B} Blocker Improves the Cognitive Performance of Mice, Rats, and Rhesus Monkeys (1993) Behavioral and Neural Biology 60:62-68.

Morgan,A. E., Dewey,S. L. Effects of pharmacologic increases in brain GABA levels on cocaine-induced changes in extracellular dopamine. Synapse, 28:60-65.

Mugnaini, E., Oertel, W. H. An atlas of the distribution of GABAergic neurons and terminals in the rat CNS as revelaed by GAD immunocytochemistry. In A. Bjorklund and T. Hokfelt (EDS.), Handbook of Chemical Neuroanatomy, vol.4: GABA and Neuropeptides in the CNS Part 1, Elsevier, Amsterdam, 1985, pp. 436-608.

Nielsen, H., Engelbrecht, J., Brunak, S., von Heijne, G. Identification of prokaryotic and eukaryotic signal peptides and prediction of their cleavage sites. (1997) Protein Engineering 10: 1-6.

Nimchinsky, E. A., Hof, P. R., Janssen, W. G. M., Morrison, J. H., Schmauss, C. Expression of dopamine D3 receptor dimers and tetramers in brain and in transfected cells. (1997) J.Biol.Chem. 272:29229-29237.

North, R. A. (1989). Drug receptors and the inhibition of nerve cells. Br. J. Pharmacol. 98:13-23.

O'Hara, P. J., Sheppard, P. O., Hogersen, H., et al. (1998) The ligand-binding domain in metabotropic glutamate receptors is related to bacterial periplasmic binding proteins. Neuron 11:41-52.

Paxinos, G., Watson, C. (Eds.), The Rat Brain in Stereotaxic Coordinates. Academic Press, San Diego, 1998.

Roberts, D. C. S., Andrews, M. M., Vickers, G. J. (1996) Baclofen attenuates the reinforcing effects of cocaine in rats. Neuropsychopharmacology 15:417-423.

Romano, C., Yang, W. L., O'Malley, K. L. (1996) Metabotropic glutamate receptor 5 is a disulfide-linked dimer. J. Biol. Chem. 271:28612-28616.

Salon, J. A., Owicki, J. C. Real-time measurements of receptor activity: applications of microphysiometric techniques to receptor biology, in Methods in Neuroscience Vol. 25, 201-223 (Academic Press, 1995).

Sambrook, J., Fritsch, E. F. and Maniatis, T. (1989). Molecular cloning: a laboratory manual (Cold Spring Harbor Laboratory Press, Cold Spring Harbor, N.Y.), 2nd Ed.

Santicioli, P., Maggi, C. A., Meli, A. (1986). The postganglionic excitatory innervation of the mouse urinary bladder and its modulation by prejunctional GABAB receptors. J. Auton. Pharmacol. 6:53-66.

Sawynok, J. (1986). Baclofen activates two distinct receptors in the rat spinal cord and guinea pig ileum. Neuropharmacology 25:795-798.

Sawynok, J. (1987) GABAergic mechanisms of analgesia: an update. Pharmacol Biochem Behav 26:463-474.

Seabrook, G. R., Howson, W., Lacey, M. G. Electrophysiological characterization of potent agonists and antagonists at pre- and postsynaptic GABAB receptors on neurones in rat brain slices. (1990) Br.J.Pharmacol. 101:949-957.

Waidmeier, P. C., Wicki, P., Feldtrauer, J. J., Mickel, S. J., Bittiger, H., Baumann, P. A. (1994). GABA and glutamate release affected by GABA_{B} receptor antagonists with similar potency: no evidence for pharmacologically different presynaptic receptors. Br. J. Pharmacol. 113:1515-1521.

Werner, P., Hussy, N., Buell, G., Jones, K. A., North, R. A. (1996) D2, D3 and D4 dopamine receptors couple to G protein-regulated potassium channels in Xenopus oocytes. Mol. Pharmacol. 49:656-661.

Wisden, W., Seeburg, P. H. (1993) Mammalian ionotropic glutamate receptors. Cur. Opin. Neurobiol. 3:291-298.

Wreggett, K. A., Wells, J. W. Cooperativity manifest in the binding properties of purified cardiac muscarinic receptors. (1995)_J.Biol.Chem. 270:22488-22499.

What is claimed is:

1. An isolated nucleic acid encoding a GABA_{B}R2 polypeptide.

2. The nucleic acid of claim 1, wherein the nucleic acid is DNA.

3. The DNA of claim 2, wherein the DNA is cDNA.

4. The DNA of claim 2, wherein the DNA is genomic DNA.

5. The nucleic acid of claim 1, wherein the nucleic acid is RNA.

6. The nucleic acid of claim 1, wherein the nucleic acid encodes a mammalian GABA_{B}R2 polypeptide.

7. The nucleic acid of claim 1, wherein the nucleic acid encodes a rat GABA_{B}R2 polypeptide.

8. The nucleic acid of claim 1, wherein the nucleic acid encodes a human GABA_{B}R2 polypeptide.

9. The nucleic acid of claim 6, wherein the nucleic acid encodes a polypeptide characterized by an amino acid sequence in the transmembrane regions which has an identity of 90% or higher to the amino acid sequence in the transmembrane regions of the human GABA_{B}R2 polypeptide shown in FIGS. 5A-5D.

10. The nucleic acid of claim 6, wherein the nucleic acid encodes a mammalian GABA_{B}R2 polypeptide which has substantially the same amino acid sequence as does the GABA_{B}R2 polypeptide encoded by the plasmid BO-55 (ATCC Accession No. 209104).

11. The nucleic acid of claim 7, wherein the nucleic acid encodes a rat GABA_{B}R2 polypeptide which has an amino acid sequence encoded by the plasmid BO-55 (ATCC Accession No. 209104).

12. The nucleic acid of claim 7, wherein the nucleic acid encodes a rat GABA_{B}R2 polypeptide having substantially the same amino acid sequence as the amino acid sequence shown in FIGS. 4A-4D (SEQ ID NO:4).

13. The nucleic acid of claim 7, wherein the rat GABA_{B}R2 polypeptide has an amino acid sequence which comprises the amino acid sequence shown in FIGS. 4A-4D (SEQ ID NO:4).

14. The nucleic acid of claim 6, wherein the nucleic acid encodes a mammalian GABA_{B}R2 polypeptide which has substantially the same amino acid sequence as does the GABA_{B}R2 polypeptide encoded by the plasmid pEXJT3T7-hGABAB2 (ATCC Accession No. 203515).

15. The nucleic acid of claim 8, wherein the human GABA_{B}R2 polypeptide comprises an amino acid sequence substantially the same as the amino acid sequence encoded by plasmid pEXJT3T7-hGABAB2 (ATCC Accession No. 203515).

16. The nucleic acid of claim 8, wherein the human GABA_{B}R2 polypeptide comprises an amino acid sequence substantially the same as the amino acid sequence in FIGS. 23A-23D (SEQ ID NO:47).

17. The nucleic acid of claim 8, wherein the human GABA_{B}R2 polypeptide has an amino acid sequence which comprises the sequence shown in FIGS. 23A-23D (SEQ ID NO:47).

18. A purified GABA_{B}R2 protein.

19. A vector comprising the nucleic acid of claim 1.

20. A vector comprising the nucleic acid of claim 8.

21. A vector of claim 19 adapted for expression in a bacterial cell which comprises the regulatory elements necessary for expression of the nucleic acid in the bacterial cell operatively linked to the nucleic acid encoding a GABA_{B}R2 polypeptide so as to permit expression thereof.

22. A vector of claim 19 adapted for expression in an amphibian cell which comprises the regulatory elements necessary for expression of the nucleic acid in the amphibian cell operatively linked to the nucleic acid encoding a GABA_{B}R2 polypeptide so as to permit expression thereof.

23. A vector of claim 19 adapted for expression in a yeast cell which comprises the regulatory elements necessary for expression of the nucleic acid in the yeast cell operatively linked to the nucleic acid encoding a GABA_{B}R2 polypeptide so as to permit expression thereof.

24. A vector of claim 19 adapted for expression in an insect cell which comprises the regulatory elements necessary for expression of the nucleic acid in the insect cell operatively linked to the nucleic acid encoding the GABA_{B}R2 polypeptide so as to permit expression thereof.

25. A vector of claim 24 which is a baculovirus.

26. A vector of claim 19 adapted for expression in a mammalian cell which comprises the regulatory elements necessary for expression of the nucleic acid in the mammalian cell operatively linked to the nucleic acid encoding a GABA_{B}R2 polypeptide so as to permit expression thereof.

27. A vector of claim 19 wherein the vector is a plasmid.

28. The plasmid of claim 27 designated BO-55 (ATCC Accession No. 209104).

29. The plasmid of claim 27 designated pEXJT3T7-hGABAB2 (ATCC Accession No. 203515).

30. A method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within one of the two strands of the nucleic acid encoding the GABA_{B}R2 polypeptide contained in plasmid BO-55, and detecting hybridization of the probe to the nucleic acid.

31. A method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within (a) the nucleic acid sequence shown in FIGS. 22A-22D (SEQ ID NO:46) or (b) the reverse complement to the nucleic acid sequence shown in FIGS. 22A-22D (SEQ ID NO:46), and detecting hybridization of the probe to the nucleic acid.

32. A method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within one of the two strands of the nucleic acid encoding the GABA_{B}R2 polypeptide contained in plasmid pEXJT3T7-hGABAB2, and detecting hybridization of the probe to the nucleic acid.

33. A method of detecting a nucleic acid encoding a GABA_{B}R2 polypeptide, which comprises contacting the nucleic acid with a probe comprising at least 15 nucleotides, which probe specifically hybridizes with the nucleic acid encoding the GABA_{B}R2 polypeptide, wherein the probe has a unique sequence, which sequence is present within (a) the nucleic acid sequence shown in FIGS. 3A-3D (SEQ ID NO:3) or (b) the reverse complement to the nucleic acid sequence shown in FIGS. 3A-3D (SEQ ID NO:3), and detecting hybridization of the probe to the nucleic acid.

34. The method of any one of claims 30 to 33, wherein the nucleic acid is DNA.

35. The method of any one of claims 30 to 33, wherein the nucleic acid is RNA.

36. The method of any one of claims 30 to 33, wherein the probe comprises at least 15 nucleotides complementary to a unique segment of the sequence of the nucleic acid molecule encoding the GABA_{B}R2 polypeptide.

37. A method of detecting a nucleic acid encoding a GABA_{B}R2 polypeptide, which comprises contacting the nucleic acid with a probe comprising a nucleic acid of at least 15 nucleotides which is complementary to the antisense sequence of a unique segment of the sequence of the nucleic acid encoding the GABA_{B}R2 polypeptide, and detecting hybridization of the probe to the nucleic acid.

38. A method of inhibiting translation of mRNA encoding a GABA_{B}R2 polypeptide which comprises contacting such mRNA with an antisense oligonucleotide having a sequence capable of specifically hybridizing to the mRNA of claim 5, so as to prevent translation of the mRNA.

39. A method of inhibiting translation of mRNA encoding a GABA_{B}R2 polypeptide which comprises contacting such mRNA with an antisense oligonucleotide having a sequence capable of specifically hybridizing to the genomic DNA of claim 4.

40. The method of claim 38 or 39, wherein the oligonucleotide comprises chemically modified nucleotides or nucleotide analogues.

41. An isolated antibody capable of binding to a GABA_{B}R2 polypeptide encoded by the nucleic acid of claim 1.

42. The antibody of claim 41, wherein the GABA_{B}R2 polypeptide is a human GABA_{B}R2 polypeptide.

43. An antibody capable of competitively inhibiting the binding of the antibody of claim 41 to a GABA_{B}R2 polypeptide.

44. An antibody of claim 41, wherein the antibody is a monoclonal antibody.

45. A monoclonal antibody of claim 44 directed to an epitope of a GABA_{B}R2 polypeptide present on the surface of a GABA_{B}R2 polypeptide expressing cell.

46. A method of claim 38 or 39, wherein the oligonucleotide is coupled to a substance which inactivates mRNA.

47. A method of claim 46, wherein the substance which inactivates mRNA is a ribozyme.

48. A pharmaceutical composition which comprises an amount of the antibody of claim 41 ef fective to block binding of a ligand to the GABA_{B}R2 polypeptide and a pharmaceutically acceptable carrier.

49. A transgenic, nonhuman mammal expressing DNA encoding a GABA_{B}R2 polypeptide of claim 1.

50. A transgenic, nonhuman mammal comprising a homologous recombination knockout of the native GABA_{B}R2 polypeptide.

51. A transgenic, nonhuman mammal whose genome comprises antisense DNA complementary to DNA encoding a GABA_{B}R2 polypeptide of claim 1 so placed as to be transcribed into antisense mRNA which is complementary to mRNA encoding such GABA_{B}R2 polypeptide and which hybridizes to such mRNA encoding such GABA_{B}R2 polypeptide, thereby reducing its translation.

52. The transgenic, nonhuman mammal of claim 49 or 50, wherein the DNA encoding the GABA_{B}R2 polypeptide additionally comprises an inducible promoter.

53. The transgenic, nonhuman mammal of claim 49 or 50, wherein the DNA encoding the GABA_{B}R2 polypeptide additionally comprises tissue specific regulatory elements.

54. A transgenic, nonhuman mammal of any one of claims 49, 50 or 51, wherein the transgenic, nonhuman mammal is a mouse.

55. A method of detecting the presence of a GABA_{B}R2 polypeptide on the surface of a cell which comprises contacting the cell with the antibody of claim 41 under conditions permitting binding of the antibody to the polypeptide, detecting the presence of the antibody bound to the cell, and thereby detecting the presence of a GABA_{B}R2 polypeptide on the surface of the cell.

56. A method of preparing the purified GABA_{B}R2 polypeptide of claim 18 which comprises:
a. inducing cells to express a GABA_{B}R2 polypeptide;
b. recovering the polypeptide so expressed from the induced cells; and
c. purifying the polypeptide so recovered.

57. A method of preparing the purified GABA_{B}R2 polypeptide of claim 18 which comprises:
a. inserting a nucleic acid encoding the GABA_{B}R2 polypeptide into a suitable vector;
b. introducing the resulting vector in a suitable host cell;
c. placing the resulting cell in suitable condition permitting the production of the GABA_{B}R2 polypeptide;
d. recovering the polypeptide produced by the resulting cell; and
e. isolating or purifying the polypeptide so recovered.

58. A GABA_{B}R1/R2 receptor comprising two polypeptides, one of which is a GABA_{B}R2 polypeptide and another of which is a GABA_{B}R1 polypeptide.

59. A method of forming a GABA_{B}R1/R2 receptor which comprises inducing cells to express both a GABA_{B}R1 polypeptide and a GABA_{B}R2 polypeptide.

60. An antibody capable of binding to a GABA_{B}R1/R2 receptor, wherein the GABA_{B}R2 polypeptide is encoded by the nucleic acid of claim 1.

61. The antibody of claim 60, wherein the GABA_{B}R2 polypeptide is a human GABA_{B}R2 polypeptide.

62. An antibody capable of competitively inhibiting the binding of the antibody of claim 60 to a GABA_{B}R1/R2 receptor.

63. An antibody of claim 60, wherein the antibody is a monoclonal antibody.

64. A monoclonal antibody of claim 63 directed to an epitope of a GABA_{B}R1/R2 receptor present on the surface of a GABA_{B}R1/R2 polypeptide expressing cell.

65. A pharmaceutical composition which comprises an amount of the antibody of claim 60 effective to block binding of a ligand to the GABA_{B}R1/R2 receptor and a pharmaceutically acceptable carrier.

66. A transgenic, nonhuman mammal expressing a GABA_{B}R1/R2 receptor, which is not naturally expressed by the mammal.

67. A transgenic, nonhuman mammal comprising a homologous recombination knockout of the native GABA_{B}R1/R2 receptor.

68. A transgenic, nonhuman mammal of claim 66 or 67, wherein the transgenic nonhuman mammal is a mouse.

69. A method of detecting the presence of a GABA_{B}R1/R2 receptor on the surface of a cell which comprises contacting the cell with the antibody of claim 60 under conditions permitting binding of the antibody to the receptor, detecting the presence of the antibody bound to the cell, and thereby detecting the presence of a GABA_{B}R1/R2 receptor on the surface of the cell.

70. A method of determining the physiological effects of varying levels of activity of GABA_{B}R1/R2 receptors which comprises producing a transgenic nonhuman mammal of claim 66 whose levels of GABA_{B}R1/R2 receptor activity vary due to the presence of an inducible promoter which regulates GABA_{B}R1/R2 receptor expression.

71. A method of determining the physiological effects of varying levels of activity of GABA_{B}R1/R2 receptors which comprises producing a panel of transgenic nonhuman mammals of claim 66, each expressing a different amount of GABA_{B}R1/R2 receptor.

72. A method for identifying an antagonist capable of alleviating an abnormality, by decreasing the activity of a GABA_{B}R1/R2 receptor comprising administering a compound to the transgenic nonhuman mammal of claim 66 or 68, and determining whether the compound alleviates the physical and behavioral abnormalities displayed by the transgenic, nonhuman mammal, the alleviation of the abnormality identifying the compound as the antagonist.

73. An antagonist identified by the method of claim 72.

74. A pharmaceutical composition comprising an antagonist of claim 73 and a pharmaceutically acceptable carrier.

75. A method of treating an abnormality in a subject wherein the abnormality is alleviated by decreasing the activity of a GABA_{B}R1/R2 receptor which comprises administering to a subject an effective amount of the pharmaceutical composition of claim 74, thereby treating the abnormality.

76. A method for identifying an agonist capable of alleviating an abnormality, by increasing the activity of a GABA_{B}R1/R2 receptor comprising administering a compound to the transgenic nonhuman mammal of claim 66 or 68, and determining whether the compound alleviates the physical and behavioral abnormalities displayed by the transgenic, nonhuman mammal, the alleviation of the abnormality identifying the compound as the agonist.

77. An agonist identified by the method of claim 76.

78. A pharmaceutical composition comprising an agonist of claim 76 and a pharmaceutically acceptable carrier.

79. A method for treating an abnormality in a subject wherein the abnormality is alleviated by increasing the activity of a GABA_{B}R 1/R2 receptor which comprises administering to a subject an effective amount of the pharmaceutical composition of claim 78, thereby treating the abnormality.

80. A cell which expresses on its surface a mammalian GABA_{B}R1/R2 receptor that is not naturally expressed on the surface of such cell.

81. A cell of claim 80, wherein the mammalian GABA_{B}R1/R2 receptor comprises two polypeptides, one of which is a GABA_{B}R2 polypeptide and another of which is a GABA_{B}R1 polypeptide.

82. A process for identifying a chemical compound which specifically binds to a GABA_{B}R1/R2 receptor which comprises contacting cells containing nucleic acid encoding and expressing on their cell surface the GABA_{B}R1/R2 receptor, wherein such cells do not normally express the GABA_{B}R1/R2 receptor, with the compound under conditions suitable for binding, and detecting specific binding of the chemical compound to the GABA_{B}R1/R2 receptor.

83. A process for identifying a chemical compound which specifically binds to a GABA_{B}R1/R2 receptor which comprises contacting a membrane fraction from a cell extract of cells containing nucleic acid encoding and expressing on their cell surface the GABA_{B}R1/R2 receptor, wherein such cells do not normally express the GABA_{B}R1/R2 receptor, with the compound under conditions suitable for binding, and detecting specific binding of the chemical compound to the GABA_{B}R1/R2 receptor.

84. The process of claim 82 or 83, wherein the GABA_{B}R 1/R2 receptor is a mammalian GABA_{B}R1/R2 receptor.

85. The process of claim 82 or 83, wherein the GABA_{B}R1/R2 receptor comprises a GABA_{B}R 2 polypeptide which has substantially the same amino acid sequence as that encoded by the plasmid BO-55 (ATCC Accession No. 209104).

86. The process of claim 82 or 83, wherein the GABA_{B}R1/R2 receptor comprises a GABA_{B}R2 polypeptide which has substantially the same sequence as the amino acid sequence shown in FIGS. 23A-23D (SEQ ID NO:47).

87. The process of claim 82 or 83, wherein the GABA_{B}R1/R2 receptor comprises a GABA_{B}R2 polypeptide which has the amino acid sequence shown in FIGS. 23A-23D (SEQ ID NO:47).

88. The process of claims 82 or 83, wherein the GABA_{B}R1/R2 receptor comprises a GABA_{B}R2 polypeptide which has substantially the same amino acid sequence as that encoded by the plasmid pEXJT3T7-hGABAB2 (ATCC Accession No. 203515).

89. The process of claim 82 or 83, wherein the GABA_{B}R1/R2 receptor comprises a GABA_{B}R2 polypeptide which has substantially the same amino acid sequence as the sequence shown in FIGS. 23A-23D (SEQ ID NO:47).

90. The process of claim 82 or 83, wherein the GABA_{B}R1/R2 receptor comprises a GABA_{B}R2 polypeptide which has the sequence shown in FIGS. 23A-23D (SEQ ID NO:47).

91. The process of claim 89, wherein the compound is not previously known to bind to a GABA_{B}R1/R2 receptor.

92. A compound identified by the process of claim 91.

93. A process of claim 89, wherein the cell is an insect cell.

94. A process of claim 89, wherein the cell is a mammalian cell.

95. A process of claim 94, wherein the cell is nonneuronal in origin.

96. A process of claim 95, wherein the nonneuronal cell is a COS-7 cell, 293 human embryonic kidney cell, a CHO cell, a NIH-3T3 cell a mouse Y1 cell or LM(tk−) cell.

97. A process of claim 94, wherein the compound is not previously known to bind to a GABA_{B}R1/R2 receptor.

98. A compound identified by the process of claim 97.

99. A process involving competitive binding for identifying a chemical compound which specifically binds to a GABA_{B}R1/R2 receptor which comprises separately contacting cells expressing on their cell surface the GABA_{B}R1/R2 receptor, wherein such cells do not normally express the GABA_{B}R1/R2 receptor, with both the chemical compound and a second chemical compound known to bind to the receptor, and with only the second chemical compound, under conditions suitable for binding of both compounds, and detecting specific binding of the chemical compound to the GABA_{B}R1/R2 receptor, a decrease in the binding of the second chemical compound to the GABA_{B}R1/R2 receptor in the presence of the chemical compound indicating that the chemical compound binds to the GABA_{B}R1/R2 receptor.

100. A process involving competitive binding for identifying a chemical compound which specifically binds to a human GABA_{B}R1/R2 receptor which comprises separately contacting a membrane fraction from a cell extract of cells expressing on their cell surface the GABA_{B}R1/R2 receptor, wherein such cells do not normally express the GABA_{B}R1/R2 receptor, with both the chemical compound and a second chemical compound known to bind to the receptor, and with only the second chemical compound, under conditions suitable for binding of both compounds, and detecting specific binding of the chemical compound to the GABA_{B}R1/R2 receptor, a decrease in the binding of the second chemical compound to the GABA_{B}R1/R2 receptor in the presence of the chemical compound indicating that the chemical compound binds to the GABA_{B}R1/R2 receptor.

101. A process of claim 99 or 100, wherein the GABA_{B}R1/R2 receptor is a mammalian GABA_{B}R1/R2 receptor.

102. The process of claim 101, wherein the GABA_{B}R1/R2 receptor comprises a GABA_{B}R2 polypeptide which has substantially the same amino acid sequence as that encoded by plasmid BO-55 (ATCC Accession No. 209104).

103. The process of claim 99 or 100, wherein the GABA_{B}R1/R2 receptor comprises a GABA_{B}R2 polypeptide which has substantially the same amino acid sequence as that shown in FIGS. 23A-23D (SEQ ID NO:47).

104. The process of claim 99 or 100, wherein the GABA_{B}R1/R2 receptor comprises a GABA_{B}R2 polypeptide which has the amino acid sequence shown in FIGS. 23A-23D (Seq. ID NO:47).

105. The process of claim 99 or 100, wherein the GABA_{B}R1/R2 receptor comprises a GABA_{B}R2 polypeptide which has substantially the same amino acid sequence as that encoded by plasmid pEXJT3T7-hGABAB2 (ATCC Accession No.203515).

106. The process of claim 99 or 100, wherein the GABA_{B}R1/R2 receptor comprises a GABA_{B}R2 polypeptide which has substantially the same amino acid sequence as the sequence shown in FIGS. 23A-23D (SEQ ID NO:47).

107. The process of claim 99 or 100, wherein the GABA_{B}R1/R2 receptor comprises a GABA_{B}R2 polypeptide which has the sequence shown in FIGS. 23A-23D (SEQ ID NO:47).

108. The process of claim 107, wherein the cell is an insect cell.

109. The process of claim 107, wherein the cell is a mammalian cell.

110. The process of claim 109, wherein the cell is nonneuronal in origin.

111. The process of claim 110, wherein the nonneuronal cell is a COS-7 cell, 293 human embryonic kidney cell, a CHO cell, a NIH-3T3 cell a mouse Y1 cell or LM(tk−) cell.

112. The process of claim 109, wherein the compound is not previously known to bind to a GABA_{B}R1/R2 receptor.

113. A compound identified by the process of claim 112.

114. A method of screening a plurality of chemical compounds not known to bind to a GABA_{B}R1/R2 receptor to identify a compound which specifically binds to the GABA_{B}R1/R2 receptor, which comprises
(a) contacting cells containing nucleic acid encoding and expressing on their cell surface the GABA_{B}R1/R2 receptor, wherein such cells do not normally express the GABA_{B}R1/R2 receptor, with a compound known to bind specifically to the GABA_{B}R1/R2 receptor;
(b) contacting the same cells as in step (a) with the plurality of compounds not known to bind specifically to the GABA_{B}R1/R2 receptor, under conditions permitting binding of compounds known to bind the GABA_{B}R1/R2 receptor;
(c) determining whether the binding of the compound known to bind specifically to the GABA_{B}R1/R2 receptor is reduced in the presence of the plurality of the compounds, relative to the binding of the compound in the absence of the plurality of compounds, and if the binding is reduced;
(d) separately determining the extent of binding to the GABA_{B}R1/R2 receptor of each compound included in the plurality of compounds, so as to thereby identify the compound or compounds present in such plurality of compounds which specifically binds to the GABA_{B}R1/R2 receptor.

115. A method of screening a plurality of chemical compounds not known to bind to a GABA_{B}R1/R2 receptor to identify a compound which specifically binds to the GABA_{B}R1/R2 receptor, which comprises
(a) contacting a membrane fraction extract from cells containing nucleic acid encoding and expressing on their cell surface the GABA_{B}R1/R2 receptor, wherein such cells do not normally express the GABA_{B}R1/R2 receptor, with a compound known to bind specifically to the GABA_{B}R1/R2 receptor;
(b) contacting the same membrane fraction as in step (a) with the plurality of compounds not known to bind specifically to the GABA_{B}R1/R2 receptor, under conditions permitting binding of compounds known to bind the GABA_{B}R1/R2 receptor;
(c) determining whether the binding of the compound known to bind specifically to the GABA_{B}R1/R2 receptor is reduced in the presence of the plurality of compounds, relative to the binding of the compound in the absence of the plurality of compounds, and if the binding is reduced;
(d) separately determining the extent of binding to the GABA_{B}R1/R2 receptor of each compound included in the plurality of compounds, so as to thereby identify the compound or compounds present in such plurality of compounds which specifically binds to the GABA_{B}R1/R2 receptor.

116. A method of claim 114 or 115, wherein the GABA_{B}R1/R2 receptor is a mammalian GABA_{B}R1/R2 receptor.

117. A method of either of claim 114 or 115, wherein the cell is a mammalian cell.

118. A method of claim 117, wherein the mammalian cell is non-neuronal in origin.

119. The method of claim 118, wherein the non-neuronal cell is a COS-7 cell, a 293 human embryonic kidney cell, a LM(tk−) cell, a CHO cell, a mouse Y1 cell or an NIH-3T3 cell.

120. A process for determining whether a chemical compound is a GABA_{B}R1/R2 receptor agonist which comprises contacting cells with the compound under conditions permitting the activation of the GABA_{B}R1/R2 receptor, and detecting an increase in GABA_{B}R1/R2 receptor activity, so as to thereby determine whether the compound is a GABA_{B}R1/R2 receptor agonist.

121. A process for determining whether a chemical compound is a GABA_{B}R1/R2 receptor antagonist which comprises contacting cells containing nucleic acid encoding and expressing on their cell surface the GABA_{B}R1/R2 receptor, wherein such cells do not normally express the GABA_{B}R1/R2 receptor, with the compound in the presence of a known GABA_{B}R1/R2 receptor agonist, under conditions permitting the activation of the GABA_{B}R1/R2 receptor, and detecting a decrease in GABA_{B}R1/R2 receptor activity, so as to thereby determine whether the compound is a GABA_{B}R1/R2 receptor antagonist.

122. A process of claim 120 or 121, wherein the cells additionally express nucleic acid encoding GIRK1 and GIRK4.

123. A process of any one of claims 120, 121, or 122, wherein the GABA_{B}R2 receptor is a mammalian GABA_{B}R2 receptor.

124. A pharmaceutical composition which comprises an amount of a GABA_{B}R1/R2 receptor agonist determined to be an agonist by the process of claim 120 effective to increase activity of a GABA_{B}R1/R2 receptor and a pharmaceutically acceptable carrier.

125. A pharmaceutical composition of claim 124, wherein the GABA_{B}R1/R2 receptor agonist was not previously known.

126. A pharmaceutical composition which comprises an amount of a GABA_{B}R1/R2 receptor antagonist determined to be an antagonist the process of claim 121 effective to reduce activity of a GABA_{B}R1/R2 receptor and a pharmaceutically acceptable carrier.

127. A pharmaceutical composition of claim 126, wherein the GABA_{B}R1/R2 receptor antagonist was not previously known.

128. A process for determining whether a chemical compound activates a GABA_{B}R1/R2 receptor, which comprises contacting cells producing a second messenger response and expressing on their cell surface the GABA_{B}R1/R2 receptor, wherein such cells do not normally express the GABA_{B}R1/R2 receptor, with the chemical compound under conditions suitable for activation of the GABA_{B}R1/R2 receptor, and measuring the second messenger response in the presence and in the absence of the chemical compound, a change in the second messenger response in the presence of the chemical compound indicating that the compound activates the GABA_{B}R1/R2 receptor.

129. The process of claim 128, wherein the second messenger response comprises potassium channel activation and the change in second messenger is an increase in the level of potassium current.

130. A process for determining whether a chemical compound inhibits activation of a GABA_{B}R1/R2 receptor, which comprises separately contacting cells producing a second messenger response and expressing on their cell surface the GABA_{B}R1/R2 receptor, wherein such cells do not normally express the GABA_{B}R1/R2 receptor, with both the chemical compound and a second chemical compound known to activate the GABA_{B}R1/R2 receptor, and with only the second chemical compound, under conditions suitable for activation of the GABA_{B}R1/R2 receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the GABA_{B}R1/R2 receptor.

131. The process of claim 130, wherein the second messenger response comprises potassium channel activation and the change in second messenger response is a smaller increase in the level of inward potassium current in the presence of both the chemical compound and the second chemical compound than in the presence of only the second chemical compound.

132. A process of any one of claims 128, 129, 130 or 131, wherein the GABA_{B}R1/R2 receptor is a mammalian GABA_{B}R1/R2 receptor.

133. The process of claim 132, wherein the GABA_{B}R1/R2 receptor comprises a GABA_{B}R2 polypeptide which has substantially the same amino acid sequence as that encoded by the plasmid BO-55 (ATCC Accession No. 209104).

134. The process of claim 132, wherein the GABA_{B}R1/R2 receptor comprises a GABA_{B}R2 polypeptide which has substantially the same amino acid sequence as that shown in FIGS. 4A-4D (SEQ ID NO:4).

135. The process of claim 132, wherein the GABA_{B}R1/R2 receptor comprises a GABA_{B}R2 polypeptide which has substantially the same amino acid sequence as that shown in FIGS. 23A-23D (SEQ ID NO:47).

136. The process of claim 132, wherein the GABA_{B}R1/R2 receptor comprises a GABA_{B}R2 polypeptide which has the sequence, shown in FIGS. 23A-23D (SEQ ID NO:47).

137. The process of claim 132, wherein the GABA_{B}R1/R2 receptor comprises a GABA_{B}R2 polypeptide which has substantially the same amino acid sequence as that encoded by the plasmid pEXJT3T7-hGABAB2 (ATCC Accession No. 203515).

138. The process of any one of claims 128-131, wherein the cell is an insect cell.

139. The process of any one of claims 128-131, wherein the cell is a mammalian cell.

140. The process of claim 139, wherein the mammalian cell is nonneuronal in origin.

141. The process of claim 140, wherein the nonneuronal cell is a COS-7 cell, CHO cell, 293 human embryonic kidney cell, NIH-3T3 cell or LM(tk−) cell.

142. The process of claim 139, wherein the compound was not previously known to activate or inhibit a GABA_{B}R1/R2 receptor.

143. A compound determined by the process of claim 142.

144. A pharmaceutical composition which comprises an amount of a GABA_{B}R1/R2 receptor agonist determined by the process of claim 128 or 129 effective to increase activity of a GABA_{B}R1/R2 receptor and a pharmaceutically acceptable carrier.

145. A pharmaceutical composition of claim 144, wherein the GABA_{B}R1/R2 receptor agonist was not previously known.

146. A pharmaceutical composition which comprises an amount of a GABA_{B}R1/R2 receptor antagonist determined by the process of claim 130 or 131 effective to reduce activity of a GABA_{B}R1/R2 receptor and a pharmaceutically acceptable carrier.

147. A pharmaceutical composition of claim 146, wherein the GABA_{B}R1/R2 receptor antagonist was not previously known.

148. A method of screening a plurality of chemical compounds not known to activate a GABA_{B}R 1/R2 receptor to identify a compound which activates the GABA_{B}R1/R2 receptor which comprises:
(a) contacting cells containing nucleic acid encoding and expressing on their cell surface the GABA_{B}R1/R2 receptor, wherein such cells do not normally express the GABA_{B}R1/R2 receptor, with the plurality of compounds not known to activate the GABA_{B}R1/R2 receptor, under conditions permitting activation of the GABA_{B}R1/R2 receptor;
(b) determining whether the activity of the GABA_{B}R1/R2 receptor is increased in the presence of the compounds, and if it is increased;
(c) separately determining whether the activation of the GABA_{B}R1/R2 receptor is increased by each compound included in the plurality of compounds, so as to thereby identify the compound or compounds present in such plurality of compounds which activates the GABA_{B}R1/R2 receptor.

149. The process of claim 148, wherein the cells express nucleic acid encoding GIRK1 and GIRK4.

150. A method of claim 148 or 149, wherein the GABA_{B}R1/R2 receptor is a mammalian GABA_{B}R1/R2 receptor.

151. A method of screening a plurality of chemical compounds not known to inhibit the activation of a GABA_{B}R1/R2 receptor to identify a compound which inhibits the activation of the GABA_{B}R1/R2 receptor, which comprises:
(a) contacting cells containing nucleic acid encoding and expressing on their cell surface the GABA_{B}R1/R2 receptor, wherein such cells do not normally express the GABA_{B}R1/R2 receptor, with the plurality of compounds in the presence of a known GABA_{B}R1/R2 receptor agonist, under conditions permitting activation of the GABA_{B}R1/R2 receptor;
(b) determining whether the activation of the GABA_{B}R1/R2 receptor is reduced in the presence of the plurality of compounds, relative to the activation of the GABA_{B}R1/R2 receptor in the absence of the plurality of compounds, and if it is reduced;
(c) separately determining the inhibition of activation of the GABA_{B}R1/R2 receptor for each compound included in the plurality of compounds, so as to thereby identify the compound or compounds present in such a plurality of compounds which inhibits the activation of the GABA_{B}R1/R2 receptor.

152. The process of claim 151, wherein the cells express nucleic acid encoding GIRK1 and GIRK4.

153. A method of claim 151 or 152, wherein the GABA_{B}R1/R2 receptor is a mammalian GABA_{B}R1/R2 receptor.

154. A method of any one of claims 148, 149, 151, or 152, wherein the cell is a mammalian cell.

155. A method of claim 154, wherein the mammalian cell is non-neuronal in origin.

156. The method of claim 155, wherein the non-neuronal cell is a COS-7 cell, a 293 human embryonic kidney cell, a LM(tk−) cell or an NIH-3T3 cell.

157. A pharmaceutical composition comprising a compound identified by the method of claim 148 or 149, effective to increase GABA_{B}R1/R2 receptor activity and a pharmaceutically acceptable carrier.

158. A pharmaceutical composition comprising a compound identified by the method of claim 151 or 152, effective to decrease GABA_{B}R1/R2 receptor activity and a pharmaceutically acceptable carrier.

159. A process for determining whether a chemical compound is a GABA_{B}R1/R2 receptor agonist, which comprises preparing a membrane fraction from cells which comprise nucleic acid encoding and expressing on their cell surface the GABA_{B}R1/R2 receptor, wherein such cells do not normally express the GABA_{B}R1/R2 receptor, separately contacting the membrane fraction with both the chemical compound and GTPΥS, and with only GTPΥS, under conditions permitting the activation of the GABA_{B}R1/R2 receptor, and detecting GTPYS binding to the membrane fraction, an increase in GTPΥS binding in the presence of the compound indicating that the chemical compound activates the GABA_{B}R1/R2 receptor.

160. A process for determining whether a chemical compound is a GABA_{B}R1/R2 receptor antagonist, which comprises preparing a membrane fraction from cells which comprise nucleic acid encoding and expressing on their cell surface the GABA_{B}R1/R2 receptor, wherein such cells do not normally express the GABA_{B}R 1/R2 receptor, separately contacting the membrane fraction with the chemical compound, GTPΥS and a second chemical compound known to activate the GABA_{B}R1/R2 receptor, with GTPΥS and only the second compound, and with GTPΥS alone, under conditions permitting the activation of the GABA_{B}R1/R2 receptor, detecting GTPΥS binding to each membrane fraction, and comparing the increase in GTPΥS binding in the presence of the compound and the second compound relative to the binding of GTPΥS alone, to the increase in GTPΥS binding in the presence of the second chemical compound known to activate the GABA_{B}R1/R2 receptor relative to the binding of GTPΥS alone, a smaller increase in GTPΥS binding in the presence of the compound and the second compound indicating that the compound is a GABA_{B}R1/R2 receptor antagonist.

161. A process of claim 159 or 160, wherein the GABA_{B}R2 receptor is a mammalian GABA_{B}R2 receptor.

162. The process of claim 161, wherein the GABA_{B}R1/R2 receptor comprises a GABA_{B}R2 polypeptide which has substantially the same amino acid sequence as that encoded by the plasmid BO-55 (ATCC Accession No. 209104).

163. The process of claim 162, wherein the GABA_{B}R1/R2 receptor comprises a GABA_{B}R2 polypeptide which has substantially the same amino acid sequence as that shown in FIGS. 4A-4D (SEQ ID NO:4).

164. The process of claim 161, wherein the GABA_{B}R1/R2 receptor comprises a GABA_{B}R2 polypeptide which has substantially the same amino acid sequence as that encoded by the plasmid pEXJT3T7-hGABAB2 (ATCC Accession No. 203515).

165. The process of claim 161, wherein the GABA_{B}R1/R2 receptor comprises a GABA_{B}R2 polypeptide which has substantially the same amino acid sequence as that shown in FIGS. 23A-23D (SEQ ID NO:47).

166. The process of claim 161, wherein the GABA_{B}R1/R2 receptor comprises a GABA_{B}R2 polypeptide which has the sequence shown in FIGS. 23A-23D (SEQ ID NO:47).

167. The process of claim 159 or 160, wherein the cell is an insect cell.

168. The process of claim 159 or 160, wherein the cell is a mammalian cell.

169. The process of claim 168, wherein the mammalian cell is nonneuronal in origin.

170. The process of claim 169, wherein the nonneuronal cell is a COS-7 cell, CHO cell, 293 human embryonic kidney cell, NIH-3T3 cell or LM(tk−) cell.

171. The process of claim 170, wherein the compound was not previously known to be an agonist or antagonist of a GABA_{B}R1/R2 receptor.

172. A compound determined to be an agonist or antagonist of a GABA_{B}R1/R2 receptor by the process of claim 171.

173. A method of treating spasticity in a subject which comprises administering to the subject an amount of a compound which is an agonist of a GABA_{B}R1/R2 receptor effective to treat spasticity in the subject.

174. A method of treating asthma in a subject which comprises administering to the subject an amount of a compound which is a GABA_{B}R1/R2 receptor agonist effective to treat asthma in the subject.

175. A method of treating incontinence in a subject which comprises administering to the subject an amount of a compound which is a GABA_{B}R1/R2 receptor agonist effective to treat incontinence in the subject.

176. A method of decreasing nociception in a subject which comprises administering to the subject an amount of a compound which is a GABA_{B}R1/R2 receptor agonist effective to decrease nociception in the subject.

177. A use of a GABA_{B}R2 agonist as an antitussive agent which comprises administering to the subject an amount of a compound which is a GABA_{B}R1/R2 receptor agonist effective as an antitussive agent in the subject.

178. A method of treating drug addiction in a subject which comprises administering to the subject an amount of a compound which is a GABA_{B}R1/R2 receptor agonist effective to treat drug addiction in the subject.

179. A method of treating Alzheimer's disease in a subject which comprises administering to the subject an amount of a compound which is a GABA_{B}R1/R2 receptor antagonist effective to treat Alzheimer's disease in the subject.

182. A process for making a composition of matter which specifically binds to a GABA_{B}R1/R2 receptor which comprises identifying a chemical compound using the process of any of claims, 82, 83, 99, 100, 114 or 115 and then synthesizing the chemical compound or a novel structural and functional analog or homolog thereof.

183. A process for making a composition of matter which specifically binds to a GABA_{B}R1/R2 receptor which comprises identifying a chemical compound using the process of any of claims 120, 128, or 148 and then synthesizing the chemical compound or a novel structural and functional analog or homolog thereof.

184. A process for making a composition of matter which specifically binds to a GABA_{B}R1/R2 receptor which comprises identifying a chemical compound using the process of any of claims 121, 130, or 151 and then synthesizing the chemical compound or a novel structural and functional analog or homolog thereof.

185. The process of any of claims 182, 183, or 184, wherein the GABA_{B}R1/R2 receptor is a human GABA_{B}R1/R2 receptor.

186. A process for preparing a pharmaceutical composition which comprises admixing a pharmaceutically acceptable carrier and a pharmaceutically acceptable amount of a chemical compound identified by the process of any of claims 82, 83, 99, 100, 114 or 115 or a novel structural and functional analog or homolog thereof.

187. A process for preparing a pharmaceutical composition which comprises admixing a pharmaceutically acceptable carrier and a pharmaceutically acceptable amount of a chemical compound identified by the process of any of claims 120, 128, or 148 or a novel structural and functional analog or homolog thereof.

188. A process for preparing a pharmaceutical composition which comprises admixing a pharmaceutically acceptable carrier and a pharmaceutically acceptable amount of a chemical compound identified by the process of any of claims 121, 130, or 151 or a novel structural and functional analog or homolog thereof.

189. The process of any of claims 186, 187, or 188, wherein the GABA_{B}R1/R2 receptor is a human GABA_{B}R1/R2. receptor.
